(19) United States
(12) Patent Application Publication
(10) Pub. No.: US 2026/0130943 A1
(43) Pub. Date: May 14, 2026

(54) METHODS AND COMPOSITIONS FOR TRANSGENE DELIVERY AND/OR RECONSTITUTING MICROGLIA

(71) Applicants: The Children's Medical Center Corporation, Boston, MA (US); Dana-Farber Cancer Institute, Inc., Boston, MA (US)

(72) Inventors: Alessandra Biffi, Boston, MA (US); Annita Montepeloso, Boston, MA (US); Pietro Genovese, Boston, MA (US)

(73) Assignees: The Children's Medical Center Corporation, Boston, MA (US); Dana-Farber Cancer Institute, Inc., Boston, MA (US)

(21) Appl. No.: 18/945,272

(22) Filed: Nov. 12, 2024

Related U.S. Application Data
(63) Continuation of application No. PCT/US2023/022029, filed on May 12, 2023.
(60) Provisional application No. 63/341,927, filed on May 13, 2022.

(51) Int. Cl. A61K 35/28; A61P 25/28; C12N 9/22; C12N 15/11; C12N 15/85

(52) U.S. Cl. CPC A61K 35/28; A61P 25/28; C12N 9/22; C12N 15/111; C12N 15/85; C12N 2310/20

(57) ABSTRACT

The present disclosure features CX3CR1 haploinsufficient HSPC and methods of using such cells for the treatment of a metabolic or neurological disorder. The disclosed methods include methods for making and modifying CX3CR1 haploinsufficient HSPC. Other disclosed methods include methods of treating a subject having or suspected of having a metabolic or neurological disease comprising administering to the subject a composition comprising CX3CR1 haploinsufficient HSPC.

### CROSS-REFERENCE TO RELATED APPLICATIONS

This application is a continuation under 35 U.S.C. § 111(a) of PCT International Patent Application No. PCT/US2023/022029, May 12, 2023, designating the United States and published in English, which claims priority to and the benefit of U.S. Provisional Application No. 63/341,927, filed May 13, 2022, the entire contents of each of which are incorporated by reference herein.

### SEQUENCE LISTING

The present application contains a Sequence Listing which has been submitted electronically in XML format. The entire contents of the electronic XML Sequence Listing, (Date of creation: Jun. 27, 2023; Size: 81,567 bytes; Name: 167705-018001_PCT_SL.xml), is incorporated by reference herein.

### BACKGROUND OF THE INVENTION

Recent findings indicate that hematopoietic stem and progenitor cells (HSPCs) can contribute to the turnover of resident brain myeloid cell populations upon administration of a conditioning regimen. In the context of metabolic and neurological diseases, engrafted cells can act as vehicles to deliver neuroprotective agents to the brains of affected patients. However, this approach has not been extensively adopted for the treatment of neurological and metabolic diseases due to the slow engraftment and expansion of the transplanted HSPCs and their progeny, as compared with the rapid progression of the neurological disease. Thus, there is a need for improved methods of HSPC transplantation. This disclosure is directed to this and other important needs.

### SUMMARY OF THE INVENTION

As described below, the present disclosure features methods and compositions directed to improved transgene delivery to hematopoietic stem progenitor cells and enhanced engraftment of transplanted hematopoietic stem progenitor cells and their progeny in a subject in need thereof.

In one aspect, the present disclosure provides a promoterless splice trapping cassette polynucleotide including from 5′ to 3′ a first homology arm derived from an intron or exon of a target gene, a splice acceptor site (SA), a polynucleotide encoding a heterologous polypeptide, and a second homology arm derived from an intron or exon of said target gene.

In another aspect, the present disclosure provides a vector including from 5′ to 3′ a first homology arm derived from a target gene, a spleen focus forming virus promoter, a polynucleotide encoding a heterologous polypeptide, and a second homology arm derived from the target gene.

In another aspect, the present disclosure provides a system for editing the genome of a cell. The system includes a polynucleotide including a promoterless splice trapping cassette polynucleotide comprising from 5′ to 3′ a first homology arm derived from an intron or exon of a target gene, a splice acceptor site (SA), a polynucleotide encoding a heterologous polypeptide, and a second homology arm derived from an intron or exon of said target gene, a Cas polypeptide or a polynucleotide encoding said Cas polypeptide, and a sgRNA that directs binding of the Cas to the target gene.

In another aspect, the present disclosure provides a method for enhancing engraftment of a hematopoietic stem cell or progenitor thereof expressing a therapeutic polypeptide in a subject. The method includes: a) contacting the hematopoietic stem cell or progenitor thereof with a system and b) administering said hematopoietic stem cell or progenitor thereof of a) to a subject that has undergone myeloablation. The system includes: (i) a promoterless splice trapping cassette polynucleotide including from 5′ to 3′ a first homology arm derived from an intron or exon of a CX3CR1 target gene, a splice acceptor site (SA), a polynucleotide encoding a heterologous polypeptide, and a second homology arm derived from an intron or exon of said CX3CR1 target gene; (ii) a Cas polypeptide or a polynucleotide encoding said Cas polypeptide, and (iii) a sgRNA that directs binding of the Cas to the CX3CR1 target gene, where contact with the system inserts the heterologous polypeptide into the CX3CR1 target gene, thereby generating a CX3CR1 haploinsufficient hematopoietic stem cell expressing a heterologous polypeptide.

In another aspect, the present disclosure provides a method of treating a neurometabolic or a neurologic, or neurodegenerative disease in a subject in need thereof. The method includes: a) contacting the hematopoietic stem cell or progenitor thereof with a system and b) administering said hematopoietic stem cell or progenitor thereof of a) to a subject that has undergone myeloablation. The system includes: (i) a promoterless splice trapping cassette polynucleotide including from 5′ to 3′ a first homology arm derived from an intron or exon of a CX3CR1 target gene, a splice acceptor site (SA), a polynucleotide encoding a heterologous polypeptide, and a second homology arm derived from an intron or exon of said CX3CR1 target gene; (ii) a Cas polypeptide or a polynucleotide encoding said Cas polypeptide, and (iii) a sgRNA that directs binding of the Cas to the CX3CR1 target gene, where contact with the system inserts the heterologous polypeptide into the CX3CR1 target gene, thereby generating a CX3CR1 haploinsufficient hematopoietic stem cell expressing a heterologous polypeptide.

In another aspect, the present disclosure provides a sgRNA including the following sequences UGAUUCAGGGAACUGAUCCA (SEQ ID NO: 1), ACUAUAGGGCUGGUAAUCGU (SEQ ID NO: 2), or GUCACCAAUCCUGUCCCUAG (SEQ ID NO: 3).

In any of the above aspects, or embodiments thereof, the target gene encodes a CX3CR1 polypeptide.

In any of the above aspects, or embodiments thereof, the intron is intron 4 of a CX3CR1 polynucleotide.

In any of the above aspects, or embodiments thereof, the exon is exon 5 of a CX3CR1 polynucleotide.

In any of the above aspects, or embodiments thereof, the heterologous polynucleotide encodes a therapeutic polypeptide.

In any of the above aspects, or embodiments thereof, the therapeutic polypeptide is: a lysosomal polypeptide associated with lysosomal disorders; a polypeptide associated with peroxisomal diseases; a microglia-associated polypeptide and/or receptor; a neuromodulating polypeptide; or a polypeptide associated with the pathogenesis of neurodegenerative disorders.

In any of the above aspects, or embodiments thereof, the first homology arm and the second homology arm each include at least about 250-1000 base pairs of a target gene intron or exon.

In another aspect, the present disclosure also provides a vector including the promoterless splice trapping cassette of any of the above aspects or embodiments thereof.

In any of the above aspects, or embodiments thereof, the vector is a viral vector.

In any of the above aspects, or embodiments thereof, the viral vector is an AAV vector.

In any of the above aspects, or embodiments thereof, the viral vector is AAV6.

In any of the above aspects, or embodiments thereof, the target gene is CX3CR1.

In any of the above aspects, or embodiments thereof, the target gene encodes a CX3CR1 polypeptide.

In any of the above aspects, or embodiments thereof, the intron is intron 4 of a CX3CR1 polynucleotide.

In any of the above aspects, or embodiments thereof, the exon is exon 5 of a CX3CR1 polynucleotide.

In any of the above aspects, or embodiments thereof, the heterologous polynucleotide encodes a therapeutic polypeptide.

In any of the above aspects, or embodiments thereof, the therapeutic polypeptide is: a lysosomal polypeptide associated with lysosomal disorders; a polypeptide associated with peroxisomal diseases; a microglia-associated polypeptide and/or receptor; a neuromodulating polypeptide; or a polypeptide associated with the pathogenesis of neurodegenerative disorders.

In any of the above aspects, or embodiments thereof, the first homology arm and the second homology arm each include at least about 250-1000 base pairs of a target gene intron or exon, and/or the first homology arm and the second homology arm are derived from sequences of the target gene intron or exon which are between less than 10 bp away to less than 100 bp away from the Cas double stranded break site.

In any of the above aspects, or embodiments thereof, the sgRNA directs binding of the Cas to a CX3CR1 polynucleotide.

In any of the above aspects, or embodiments thereof, the sgRNA includes a spacer complementary to a sequence listed in Table 2.

In any of the above aspects, or embodiments thereof, the Cas polypeptide is a Cas9 nickase.

In another aspect, the present disclosure also provides a cell including the promoterless splice trapping cassette of any of the above aspects, or embodiments thereof, the vector of any of the above aspects, or embodiments thereof, or the system of any of the above aspects, or embodiments thereof.

In any of the above aspects, or embodiments thereof, the cell is a hematopoietic stem cell or progenitor thereof.

In another aspect, the present disclosure also provides a method for inserting a heterologous polynucleotide in the genome of a cell. The method includes contacting the cell with the system of any of the above aspects, or embodiments thereof, thereby inserting the heterologous polynucleotide into the genome of the cell.

In any of the above aspects, or embodiments thereof, the target gene is CX3CR1.

In any of the above aspects, or embodiments thereof, the heterologous polynucleotide encodes a therapeutic polypeptide.

In any of the above aspects, or embodiments thereof, the therapeutic polypeptide is a lysosomal polypeptide associated with lysosomal disorders, a polypeptide associated with peroxisomal diseases, a microglia-associated polypeptide and/or receptor, a neuromodulating polypeptide, or a polypeptide associated with the pathogenesis of neurodegenerative disorders.

In any of the above aspects, or embodiments thereof, the cell is a hematopoietic stem cell or progenitor thereof.

In any of the above aspects, or embodiments thereof, the intron is intron 4 of a CX3CR1 polynucleotide.

In any of the above aspects, or embodiments thereof, the exon is exon 5 of a CX3CR1 polynucleotide.

In any of the above aspects, or embodiments thereof, the subject is a human.

In any of the above aspects, or embodiments thereof, the method reduces or eliminates expression of the CX3CR1 gene in the cell.

In any of the above aspects, or embodiments thereof, the method enhances engraftment of the edited hematopoietic stem cell or progenitor thereof in bone marrow or brain of the subject relative to control hematopoietic stem cell that is not CX3CR1 haploinsufficient.

In any of the above aspects, or embodiments thereof, the therapeutic polypeptide is expressed under the control of an endogenous CX3CR1 promoter.

Compositions and methods defined in this disclosure were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “ablative conditioning” refers to administering to a subject a composition that destroys endogenous hematopoietic stem and progenitor cells in the bone marrow niche, and functionally defined microglia progenitors in the central nervous system.

By “agent” is meant any small molecule chemical compound, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, and a 50% or greater change in expression levels.

By “ameliorate” is meant decrease, suppress, attenuate, diminish, arrest, or stabilize the development or progression of a disease.

By “biologic sample” is meant any tissue, cell, fluid, or other material derived from an organism.

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CX3CR1 protein” or “human beta chemokine receptor-like 1 protein” is meant a protein having at least about 85% amino acid sequence identity to GenBank Accession No.: ABS29268.1 or a fragment thereof and is a receptor for fractalkine. An exemplary CX3CR1 polypeptide sequence is provided below.

>AAA87032.1 beta chemokine receptor-like 1
[Homo sapiens]
(SEQ ID NO: 4)
MDQFPESVTENFEYDDLAEACYIGDIVVFGTVFLSIFYSVIFAIGLVGN
LLVVFALTNSKKPKSVTDIYLLNLALSDLLFVATLPFWTHYLINEKGLH
NAMCKFTTAFFFIGFFGSIFFITVISIDRYLAIVLAANSMNNRTVQHGV
TISLGVWAAAILVAAPQFMFTKQKENECLGDYPEVLQEIWPVLRNVETN
FLGFLLPLLIMSYCYFRIIQTLFSCKNHKKAKAIKLILLVVIVFFLFWT
PYNVMIFLETLKLYDFFPSCDMRKDLRLALSVTETVAFSHCCLNPLIYA
FAGEKFRRYLYHLYGKCLAVLCGRSVHVDFSSSESQRSRHGSVLSSNFT
YHTSDGDALLLL

By “CX3CR1 polynucleotide” or “human beta chemokine receptor-like polynucleotide” is meant a nucleic acid molecule encoding an CX3CR1 polypeptide. The CX3CR1 gene encodes a receptor for fractalkine. Exemplary CX3CR1 polynucleotide sequences are provided below:

>U28934.1 Human beta chemokine receptor-like 1 mRNA, complete cds
(SEQ ID NO: 5)
GGGGCAGATCCAGATTCCCTTTGCAGTCCACGCCAGGCCTTCACCATGGATCAGTTCCCTGA
ATCAGTGACAGAAAACTTTGAGTACGATGATTTGGCTGAGGCCTGTTATATTGGGGACATCG
TGGTCTTTGGGACTGTGTTCCTGTCCATATTCTACTCCGTCATCTTTGCCATTGGCCTGGTG
GGAAATTTGTTGGTAGTGTTTGCCCTCACCAACAGCAAGAAGCCCAAGAGTGTCACCGACAT
TTACCTCCTGAACCTGGCCTTGTCTGATCTGCTGTTTGTAGCCACTTTGCCCTTCTGGACTC
ACTATTTGATAAATGAAAAGGGCCTCCACAATGCCATGTGCAAATTCACTACCGCCTTCTTC
TTCATCGGCTTTTTTGGAAGCATATTCTTCATCACCGTCATCAGCATTGATAGGTACCTGGC
CATCGTCCTGGCCGCCAACTCCATGAACAACCGGACCGTGCAGCATGGCGTCACCATCAGCC
TAGGCGTCTGGGCAGCAGCCATTTTGGTGGCAGCACCCCAGTTCATGTTCACAAAGCAGAAA
GAAAATGAATGCCTTGGTGACTACCCCGAGGTCCTTCAGGAAATCTGGCCCGTGCTCCGCAA
TGTGGAAACAAATTTTCTTGGCTTCCTACTCCCCCTGCTCATTATGAGTTATTGCTACTTCA
GAATCATCCAGACGCTGTTTTCCTGCAAGAACCACAAGAAAGCCAAAGCCATTAAACTGATC
CTTCTGGTGGTCATCGTGTTTTTCCTCTTCTGGACACCCTACAACGTTATGATTTTCCTGGA
GACGCTTAAGCTCTATGACTTCTTTCCCAGTTGTGACATGAGGAAGGATCTGAGGCTGGCCC
TCAGTGTGACTGAGACGGTTGCATTTAGCCATTGTTGCCTGAATCCTCTCATCTATGCATTT
GCTGGGGAGAAGTTCAGAAGATACCTTTACCACCTGTATGGGAAATGCCTGGCTGTCCTGTG
TGGGCGCTCAGTCCACGTTGATTTCTCCTCATCTGAATCACAAAGGAGCAGGCATGGAAGTG
TTCTGAGCAGCAATTTTACTTACCACACGAGTGATGGAGATGCATTGCTCCTTCTCTGAAGG
GAATCCCAAAGCCTTGTGTCTACAGAGAACCTGGAGTTCCTGAACCTGATGCTGACTAGTGA
GGAAGATTTTTGTTGTTATTTCTTACAGGCACAAAATGATGGACCCAATGCACACAAAACAA
CCCTAGAGTGTTGTTGAGAATTGTGCTCAAAATTTGAAGAATGAACAAATTGAACTCTTTGA
ATGACAAAGAGTAGACATTTCTCTTACTGCAAATGTCATCAGAACTTTTTGGTTTGCAGATG
ACAAAAATTCAACTCAGACTAGTTTAGTTAAATGAGGGTGGTGAATATTGTTCATATTGTGG
CACAAGCAAAAAGGGTGTCTGAGCCCTCAAAGTGAGGGGAACCAGGGCCTGAGCCAAGCTA
>CX3CR1-202 ENSE00001537013 exon: protein_coding
(SEQ ID NO: 6)
GAAATACTCGTCTCTGGTAAAGTCTGAGCAGGACAGGGTGGCTGACTGGCAGATCCAGAGGT
TCCCTTGGCAGTCCACGCCAG
>CX3CR1-202 ENSE00001777225 exon: protein_coding
(SEQ ID NO: 7)
GCCTTCACCATGGATCAGTTCCCTGAATCAGTGACAGAAAACTTTGAGTACGATGATTTGGC
TGAGGCCTGTTATATTGGGGACATCGTGGTCTTTGGGACTGTGTTCCTGTCCATATTCTACT
CCGTCATCTTTGCCATTGGCCTGGTGGGAAATTTGTTGGTAGTGTTTGCCCTCACCAACAGC
AAGAAGCCCAAGAGTGTCACCGACATTTACCTCCTGAACCTGGCCTTGTCTGATCTGCTGTT
TGTAGCCACTTTGCCCTTCTGGACTCACTATTTGATAAATGAAAAGGGCCTCCACAATGCCA
TGTGCAAATTCACTACCGCCTTCTTCTTCATCGGCTTTTTTGGAAGCATATTCTTCATCACC
GTCATCAGCATTGATAGGTACCTGGCCATCGTCCTGGCCGCCAACTCCATGAACAACCGGAC
CGTGCAGCATGGCGTCACCATCAGCCTAGGCGTCTGGGCAGCAGCCATTTTGGTGGCAGCAC
CCCAGTTCATGTTCACAAAGCAGAAAGAAAATGAATGCCTTGGTGACTACCCCGAGGTCCTC
CAGGAAATCTGGCCCGTGCTCCGCAATGTGGAAACAAATTTTCTTGGCTTCCTACTCCCCCT
GCTCATTATGAGTTATTGCTACTTCAGAATCATCCAGACGCTGTTTTCCTGCAAGAACCACA
AGAAAGCCAAAGCCATTAAACTGATCCTTCTGGTGGTCATCGTGTTTTTCCTCTTCTGGACA
CCCTACAACGTTATGATTTTCCTGGAGACGCTTAAGCTCTATGACTTCTTTCCCAGTTGTGA
CATGAGGAAGGATCTGAGGCTGGCCCTCAGTGTGACTGAGACGGTTGCATTTAGCCATTGTT
GCCTGAATCCTCTCATCTATGCATTTGCTGGGGAGAAGTTCAGAAGATACCTTTACCACCTG
TATGGGAAATGCCTGGCTGTCCTGTGTGGGCGCTCAGTCCACGTTGATTTCTCCTCATCTGA
ATCACAAAGGAGCAGGCATGGAAGTGTTCTGAGCAGCAATTTTACTTACCACACGAGTGATG
GAGATGCATTGCTCCTTCTCTGAAGGGAATCCCAAAGCCTTGTGTCTACAGAGAACCTGGAG
TTCCTGAACCTGATGCTGACTAGTGAGGAAAGATTTTTGTTGTTATTTCTTACAGGCACAAA
ATGATGGACCCAATGCACACAAAACAACCCTAGAGTGTTGTTGAGAATTGTGCTCAAAATTT
GAAGAATGAACAAATTGAACTCTTTGAATGACAAAGAGTAGACATTTCTCTTACTGCAAATG
TCATCAGAACTTTTTGGTTTGCAGATGACAAAAATTCAACTCAGACTAGTTTAGTTAAATGA
GGGTGGTGAATATTGTTCATATTGTGGCACAAGCAAAAGGGTGTCTGAGCCCTCAAAGTGAG
GGGAAACCAGGGCCTGAGCCAAGCTAGAATTCCCTCTCTCTGACTCTCAAATCTTTTAGTCA
TTATAGATCCCCCAGACTTTACATGACACAGCTTTATCACCAGAGAGGGACTGACACCCATG
TTTCTCTGGCCCCAAGGGCAAAATTCCCAGGGAAGTGCTCTGATAGGCCAAGTTTGTATCAG
GTGCCCATCCCTGGAAGGTGCTGTTATCCATGGGGAAGGGATATATAAGATGGAAGCTTCCA
GTCCAATCTCATGGAGAAGCAGAAATACATATTTCCAAGAAGTTGGATGGGTGGGTACTATT
CTGATTACACAAAACAAATGCCACACATCACCCTTACCATGTGCCTGATCCAGCCTCTCCCC
TGATTACACCAGCCTCGTCTTCATTAAGCCCTCTTCCATCATGTCCCCAAACCTGCAAGGGC
TCCCCACTGCCTACTGCATCGAGTCAAAACTCAAATGCTTGGCTTCTCATACGTCCACCATG
GGGTCCTACCAATAGATTCCCCATTGCCTCCTCCTTCCCAAAGGACTCCACCCATCCTATCA
GCCTGTCTCTTCCATATGACCTCATGCATCTCCACCTGCTCCCAGGCCAGTAAGGGAAATAG
AAAAACCCTGCCCCCAAATAAGAAGGGATGGATTCCAACCCCAACTCCAGTAGCTTGGGACA
AATCAAGCTTCAGTTTCCTGGTCTGTAGAAGAGGGATAAGGTACCTTTCACATAGAGATCAT
CCTTTCCAGCATGAGGAACTAGCCACCAACTCTTGCAGGTCTCAACCCTTTTGTCTGCCTCT
TAGACTTCTGCTTTCCACACCTGGCACTGCTGTGCTGTGCCCAAGTTGTGGTGCTGACAAAG
CTTGGAAGAGCCTGCAGGTGCTGCTGCGTGGCATAGCCCAGACACAGAAGAGGCTGGTTCTT
ACGATGGCACCCAGTGAGCACTCCCAAGTCTACAGAGTGATAGCCTTCCGTAACCCAACTCT
CCTGGACTGCCTTGAATATCCCCTCCCAGTCACCTTGTGGCAAGCCCCTGCCCATCTGGGAA
AATACCCCATCATTCATGCTACTGCCAACCTGGGGAGCCAGGGCTATGGGAGCAGCTTTTTT
TTCCCCCCTAGAAACGTTTGGAACAATCTAAAAGTTTAAAGCTCGAAAACAATTGTAATAAT
GCTAAAGAAAAAGTCATCCAATCTAACCACATCAATATTGTCATTCCTGTATTCACCCGTCC
AGACCTTGTTCACACTCTCACATGTTTAGAGTTGCAATCGTAATGTACAGATGGTTTTATAA
TCTGATTTGTTTTCCTCTTAACGTTAGACCACAAATAGTGCTCGCTTTCTATGTAGTTTGGT
AATTATCATTTTAGAAGACTCTACCAGACTGTGTATTCATTGAAGTCAGATGTGGTAACTGT
TAAATTGCTGTGTATCTGATAGCTCTTTGGCAGTCTATATGTTTGTATAATGAATGAGAGAA
TAAGTCATGTTCCTTCAAGATCATGTACCCCAATTTACTTGCCATTACTCAATTGATAAACA
TTTAACTTGTTTCCAATGTTTAGCAAATACATATTTTATAGAACTTC
>CX3CR1-202 intron 1: protein_coding
(SEQ ID NO: 8)
GTAGGTGCACAGGACTAGTTGGGTACCTGTGGGTGGGGTGGAGCAGTGGACAGCTAATAGGT
TAATAATGCCTGTTTGCTTACGTGCAGACAATGGAAACCATTTTCCTGGGGATGTTGTAGCC
TAAATATGTCCAAGGGGATGGAAGAGTGGGAGGCAAGGGGTGATCAGATCATTTATAATACA
CTCAACCTGGTGGAATAGTATTAGAAGCATTAGTAATTACATTTTAGAGACATGGAGAAAAG
CTCATGATTTTAAACTAACTGAAAAAAGCATGAAAAATTGCATCTGGATGCTGTTGAGGAAT
TATTGCTAATTTTTTGAGATGAGAAATTGTATTAAAGTCTTTTTCAAAAAAGAGTCCTTAAC
TTTTAGAGATGCACACAAGTGTTTATGGGTGAAATTAAATAATTCAGTAGAGACATTAAGTG
GGAAATAGAGGAAATATTGACCATGGGTTGCTAATAGTTGAAGCCAGGTGTTGGGTATAAGG
AGGTTCTCGCTGCTTTTATTTGAAAAGTTGCTTTTTATTTGAAAATTTAATAATAAAGAGTT
TTTAAATTTGTATCTGTATTTTAATATAAATATAAATGCACTTAATATAAATATAAAATATG
TATAACATTTAGATAGAGAAAAGCTAAAAGATTACTGTGGTTGATTCTAGGAAACTGCATTG
CAGATAGTTTCATGGGTTTTTTTTTCCTTTTTCTTCCACTTTTTGTACCATCTATGGGTTTT
TGTTTGTTTTTTGTTTTTTGGGTTTTCTTTCTTGTTTGTGTTTTGTTTTTTGAGACGGAGTT
TTGCTCTTGTTGCCTAAGCTGGAGTGCAATGGCACAGTCTCGGCTCACTGCAACCTCTGCCT
CCTGGGTTCAAGTGATTCTCCTGCCTCAGCCTCCCAAGTAGCTGGGATTATAGGCATGTACC
ACCGCCCGGCTAATTTTGTATTTTTAGTAGAGGCGGGGTTTCTCCATTAATAAATTCCTGGC
ACAAATTTAGTGTTCAATTTTGATATATGTTGTTATAACCATTGTGAGGATACTCAGGCTCA
GGTTTGTGTGGGTGGAAAACATGGTCTTCAGAAAGAAATTATGAGTGCAAGACAGGAGGAAA
TCCATCAGAGGCCCCAGCTGAGGACTGACCACGGCTTGTTATTTCTCTTGCCTTGCCTCTGG
CAATCACAGCCTCACAGAGCCTGCAATCCTTGCTTTGTGAGTTTATAGCTCAGTCCAGAGAA
TGGCTAAGAAAGTTTAGGATTCTTTCAACACCCACTCCACAAAAAAAAAAAAAAAAAAGAAA
AGAAAAAAAAATTAATTTTTGAAATACTTGAGGTAGAAAACTTGAGGCAGAAAAAAATTGAG
CCAAAAAAAAAGGAAAATTGAACCACGTGAAAGCAGGCAAGAAAGCTTGCATTGCTCAGGGC
ATCCCAGGCCCAGAGGGCGCTTTGGAGGGAGCTGGGTTTCCTGAGAGGAGGCAGGGTGGGTG
ACGGACCTGTGCTGGAGAGCCTTGAGGACCACTGTGGGTTGGGAATGGGGGCAGTGGATTGG
GGTTCAAAACCCCTGGGAATGAGAAATGGGCTCAGGAAGGCTAGGGTGGATTCTTTCATCTT
CCTCTTTGCTTGGCTTTATTTTCACAAAGGAAGGCAGGGCAGGAAATAGTCTCAGCCCAACT
TCAGTGTGGTTCTTCTTAGTGCTCAGGCTTACCTGGCACTTGCCACACCTCTGGGATGGGAG
CACCTACTATCCATCAGCCACGTGCCAGTCTCCACAAAGTCTGCTCCTGAACCCTGCTCCTC
AGCTGGCCCCACTTCACAGATGGGGACATAGGCAGCTTGGCTTTGGAATGAAGGAATGAAGT
CAGGAATGAAGTCCTGGCTCTGCACTTGGTGACTGTGCACTGGGCTTGCTAAGTCTGTTTCC
TGCTTTTAAAATGGAGATTGTCCATCAGCCTTTGAAGCCATGTAATGGGTATGTGTCAACTT
TCTGCAAGGATTAAAGGCATGGTATAGGAAGTCCCAAACACACTGCCTGACCCATCTTTGAT
GCTCAAGAAACGATATATGTTGTTGTCATGAGGAAACTGAGCCTCAGAAAGTTTGGATACCT
GAAAAACACTGACTACTATTGAATGAGGTTGTGAAGAATCCAGAGCTGTAGGGGCAGGAAAG
CAAAGAACGTATTAGAGCTGACCCAGTCAGGACGATCGTCTATCCCCTTCCTCACCCCACCC
CATCCCAGGAGGAAGCCTGCCCGGCCCTAGGCAGCTATGGCACAGTGGCAATGTCAGGTATG
GTTCTCCCTAGCCAGAGACCCTAGCCTCAAAAAACCTCCTTCTTGGGATCCAGGCATCCAAC
TGCTCCTCCCCAGCCCCAGCCTCTGACCCAGTATCCTGAGTCCAGAGACGTTTGGAACCAGC
ACCTGTAATGGAGGAGCTGAACAAGGAGGGGAACTTCTGCTGCTCCACAGCAGGTCACGGTC
ATAGGAGGGAGTGGAACCAGAATGGCAGAATCCAGATCTTGGCTGCCTTTCCCAAGGACTTG
TTCTGATTCCTAGCAGCACAGCCCAGGCATTCCGAGAAGTTGGGCTCTCTGGCATCACTCAC
TCTGCCCAGAAGAGCCAGGGGAAAGTTGGGGCTTCTAGCTGAACCTTGATCCCACCTGCCCT
CTTGAGGGGCTCAGAATCTGCTGGCTGCTTCACAGGTGGGATTCTCACGGCACGCTGGCCAC
AGCTGATGCTTCGACCCCCTCATCTTGTTTGGCCAAAGTGCAGCTTTTTAGCTTGTGAGTAA
GGAAGAAAAGCTGTATCATATGTCTTTAAACATCTTCCTAGACCACCTTTGTTTTCCCCTTA
AAGTGTGCTTAAGGAGAAAATGGAAAGTCTCTTTTCAGTGTGTTGTATTTTGTTTATTGCAA
AATACAACACACTGAAAGAACAATGTCTGGGTTAGCAAAGTATTAGATTTAATTTCCCACAT
TACTACCATCCAGGCTGAGAAAGAGAACCCCAGAAGCCTCTCTCATGTCCCTTCCTGACCAC
AGCCTCTTCCTCTCCCACAAAGAACCACAAGTCTGACTTCTATGGTGATCACTTCCTTGCTT
TTCCCTATAGTTTGACTGAATCTAAATATCATCCCTAAACAATATAGCTTAGCTTTGCCTAT
TTTTGATCTTCATATTTGGATTCATACTTTTTGGTTTGGCTCAAGACTAAACTTTTAAAATG
CATCCACGTTGTAGGAGATGTACATGCACAAAAATTTTCACGGCAACATTGTCCAAACTAGC
AACAAACTGAAAGCCACCTATGTGTGCATTAGCAGTAGAAATGGCAATAAATCATGGACTAG
TCACACAGTGGAATCCTACGCAGCAAGGAGAATTAGCAGTCTACAGCCAAACCAACAGCATG
GGTGAGATGCTTCCAGAAATACTGAGATGAATTTAGAGATAAAGGCACAATTGGTCCATATC
TACAGGGCACTTAATCCAGTGCATCTCCACTAGACAAAATTTGTATTTCCATGAGCAAGAGT
CATTTGCACTGCTATCTGCTATAACCTACGGTGCTGTAAAATTAGCTCAGGCAATAAAAGGG
GGAGGTAGCCCCAAAGAGTATGCCAGAAAGGACTCCCAGTAATCTTCAGTGTGTGTCTATTT
CAAAGTCTTTCATCATTTTTCCTTGTAGTTACTTAGTGTGGAATTTCAGACCCCTCTCTATG
TCCCTCTTCTCCCTTTTCAGCCTTGGCTTTCTCTGCATCCTTCCCCGAAGCCCTGCACTCAG
CCTAAACTGACTTTCCTGAACACTCTGGGAGCATGTGGGCTCCTCCCAACTCCACCATCTTC
ACTGCCTTCTGTTCCTATTTCCCTCTGCCTGGCCTTGGCCCCAGGAAACCTTCCTATGCTCA
GACCCTCTGTGCCTTTGTCTTCAAAGCCCACCCACTATTCACTGCCACCTCCATCTTGGTCA
ACCTGGAAGACTTTCCCCATCCTTAAAACATCAGCTCAAACGGCAGATTTTTTTTTTTTTTT
TTGTGATATCCTCTTGATCACCCTCCCCCAGTTTTTAAGGAGACAGATATAGAGGTTCAATC
CTTGGGCTTCCCATGACTTTTCTTATATTCTGTTACTGAGCAATAATAAACACTTCTAGGAA
CTGTATCTTAAACACTTTTGCCTCTACAGAATCTAGCACAGTGCCTAGTATTGGTGAAATAT
AATTAACAAAGTCTTCTTTCAACACAGAGATTCTCTCCACAAAAGGAGTAGAGAAAGAACAG
TTTTATTATGGAATAAGCAGTAAACCAAAATATGCAGAGCATTATAGGCCATCTACTAAGAG
GTTGCAAGAACAGAAAGAAATCTCCCCCTTTTGTATAGCCAAGTAGATACAACCTGTTACAT
ACATGTTATCAAGGCAAACAATAACTGTTCCTCAAGTAAGAGGTCTTGCCAGCACCGTTTGC
CGTACATGGTTCACTCTAAATTTACCTGGTAATTAGGGTAACCACTTGTGTTAGCTAACTGG
CTCTACCCAGAGGAAAATCAAATTTATCTTTAAGACAAGGGGTAATTTTGCAGCACTGAGCA
AGGCTCTTCAGTTAGGCTCATACCTTCCCACAGAAACTAAGAGATAGAAGCACTATCTCCCT
TAGGTTTGTTTACATTTCAAAGAGATGACGCCCAGGTCCTTGGGAAAGACTAGCTTAGCTCA
TAAAGCTGACAAAAAGCCTATCTAGTTTCAAAAGGATTTACACATGTTCAAAGAGAGGAGAA
AGTATGTAAAAGTTTTCTAGGTGGGCACGGTGGCTCACACCTGTATTCCCAGCACTTTGAGA
GGCTGAGGCGGGTGGATCACCTGAAGTCAGGAATTTGAGATCAGCCTGGCCAACATGGTGAA
ACCTTGTCTCTACTAAAAATACAAAAATTAGCTGGGCATGGTGGTGGGCGCCTGTAATCCCA
GCTACTTGGGAGGCTGAGGCAGGAGAATCACTTGAACCCAGGAGGTGGAGGTGGCAGTGAGC
CAAGATGGCACCATTGCACTCCAGCCTGGGCGATGAGAGTGAAACTCCATGTCAAAAAAAAA
AAAAGTTTTCTAAAGTAAATGCTCTGAGAAAAAAACAGAAGGTGAGGAGATCTCTGTCCTTA
TTTTTAACAAGAATAATTAAATGTTTTTATTTTTAATTTCTACTTAGACTAGAATATGGTAC
AGATGCTCAAAAATATTGACTTAATTAATAATTAAACAATTAACAAGTCTATCTCTTTCCTA
ATTATTAAAAAAATTATGAAGTATTTCCCTTCAAAATTTCACAGTTTACTTTGACATAAATA
AATAAATACCATAAAGTATTTTGAAGAGGAAAGGGAAGGGTGGGGCTTCAAACACAAAATAA
TGGCAGGTTTTGTAAAGGGGCAATTCACTAAACCCAATAGGATTCTCCTGTTGCTATTTTTT
TTTTTTTTTTTTTTTTGGTGGTGGTTGGAGGGAAGGGTTGGGTGTGCATTTGGCCTGTGTGA
AAAACTGGTGGGTAGACAATGCCATGCTGTTCTAGATGGGCTCATTGCTATAGGAATAGATA
GTCACTGAATTTACTTGCATAGGGGATGGGGTATAAAATGTTTCCTTATAGCTCCTTAACAT
GGAATTCAGCACCCCTCTCCCCATCAGCCTTAGCTCTCTCTGAATCCCAAGGGATATGTGTT
ATAAAAATAGCTGCCATCATAAATGAGAAAACCATCAGGACCTGGTAAAACTTGGCTTCCCA
AACAGTCAGGGGTCTGGGCATGGCAGCCGACTGAGAACCTTTCTATCTAGTACAAGGTAAAC
AAGATTTGCCAAGACTTGATCAACTGATCCAGCTCCCTATCTCCAAACAGAACCTCATCTGA
ATGCTCACAGGCTGTGCCCCAGAAGTGTAGAAGTGTCATGTTCCCTGGGCAGGCTGCTGGGC
AGCCTCTCTGTTGACAATAGACCACACTTTTGCTGACCTAGGACTCATGTTGCTCTTTAAGA
CTGCTTCCTTGGCCAGGCATGATGGCTCACACCTGTAATCCTGGCACTTTGAGAGGCCCAGG
CAGGAGGATCTCTTGAGGCCAGATGTTCAAGACCAGCCTGGTCAACATAGTAAGACCCCATA
TCTACCAAAAATAGCTGGGCATGGTGGTGCACACCTATACTCCCAGCTACTTAGGAGACTGA
GGTGGGAGGATTGCTACATCCCAGGAGTTCAAGGCTGTAGTGAGCTATGATCATGCCACTGC
ACTCCAGCCTGGGCAACAGAGCGAGATCCTGTCTCAAACAAACAGTTTCCTTCTGTTTGATT
CTTGCTGAAAAATTGAGCATGCCAGAGCTAGCAAGGCTCTTAGAGGTGGTCTGGTCCAATGC
TTTAATTTCATACATCAAGAAACTGAAGCAGAGCAGAGTGACCTGCCCAGGGTCTCTCAGCC
ATTCATGCTCAGAAATGTATGGGCTCCTGTGAAACATGTGGCTCTTAAAAGCACTATCATAT
ATTTGAAGGCAGAAAATAGGCTAAACCTTCAGCCTTCAGACTTTTCCTCTCCAGAGAAAATG
ACCCCAGTTTCCTCACTATGGTTGCTGGGAGCTAGATTCCTGGGGATCTGGCAGTGTGGACC
ACCTAGTGGTGGCTAGAGGAGCAAATAATATCCCGCATTCCATTTTCCACTCACCAATCCCT
GAGGGGCAGCCTGCTGGGTTATGAGCCCACAGGGGGAGAACCCCAACGAATTCAGAGATGCA
TCATGGACCAGTTTCTCTTAAGGGGCCTGGGTCTACTATTTTCAGTTCTACTTCGAGAGAAG
TGGCCTGCAATATCCTGCAGATTTCCCCTCCAGGGAGAAAAGCATTGTGCGGTGCAAGGAGC
ACAGGCTTTGTGGAAAGAGGCATCTGGGGTTGAGATCCTGGCTCTTTTGCTTCCTTGAACAA
GTTAACCAAATCTCTGGGCCTCTGCTGGTTAATTTATACCATGGGGATCATCATTTCTCCAG
TGGGGTTGTGAAGAGAATGTGGTGGGATCTTGTGTGTGGAGCATGACACTTAGCAGGCATCC
GGGAATGGCAGCCTCCTCCCTTTCTAAACTGGGGCTTTCTGAGGGTGACTTCAGATTCCACA
ATGTCAACAGCACAATGGCATCCTCATAAGGAAAGTTTGGGTTGGGGCTCCTCAAGCAATTC
TCTACTCTCATTTGGTACAAAGAAAAAAATTAAGCCTCACAATTTTCTTGGCACCAGACTGA
ACCTCAAACCCAGTCTTCACTTTTACTAAAAAGCCATAAACAGAGACCAGGAGGGTAAAAAC
TACCAGAAGATACACTGGATTTAGAAAACAGTAAGCTGGATGTGAAGCCAAAAGAGAGGGAG
AAAGCAACATAAAAGAATGTCAGATTGAAGAAGAAATGGATTCTGGCCACCTAAAGAAGAAG
GGAAGTATAGAAAAGGGAAGCTGATTGGAAACAATGGGTAATGATGAGTGTCCCTCCCCTAA
AAAGTTAAAAAATAAATGAGCAGGCATGAGAAAAGGAAGTAGGCCAGAGGAAGTAGACCAGG
ATCAGCCACGGATGTGAGTGGGGAATTTTACAAATTTCTACTTGGGTGTGAAACATGTGTAC
ACTCAAAGCTCTGTCTAGGTTCACCGAAACTCTCCCAGCTCAAGGCCATTTAGTAAAACCTT
GGCTTAACTGAATAGTGGGTAAGGAGGCCTGAGGATGGCCCAGAGAAGGTTTAAATTCTTAC
TGGCTGCTGCACTAAGTTGTAAAGTGCAGCCTTAGTTGATGATGCCTTTATTAGTGCAACAT
CCCAGAATGCATGCGTCTTACAACCCTTGTGGATATGAAACCCAGCTGGGTAGACACTGCAA
AGTCTTCTCATTTGATTCTTCCACTTGGTTTCTAGTGTCCCTCAGGGACAAAGGAAAGCCAT
GGTCCAGTCTAAGATGAAAACCAATTAGACCTCTGGCAGGCCTTTGACCACCCTGAGGGCCA
CCCCCAAGGCACGGCCACACTCGCATCTGCTGCCAGGAGGCCCTATCTTAGGCTCAGCTCCC
AAACCTTGAACATCTTGGAGGATCCACAAATAGGAGGTACACTCAGCTCAGGCTCAGTCTTC
CTTAAAAAGCGCCTTGCTAAAGCTATCAAGTTCACTGGAATACTTCTGCGAAGGACACAGCT
TCAGCGATGTCAGATTTTTTATGTAAATGGTGCCTTCACATGCTCTGGGGCATTTGGCTACC
AAGGGCGGTTTGAACTAGCTCCAGCACACAGAATACAAGTCTCTTCAGAACCAGGCAAAACC
CTATGTTGCCCAATGACTCCTGCTGTTTCTTGAGATTTGCACAGAAGACAGAGCTGCAATTA
CCCACGCTGATATCTATTTCATGACCACATATGTTCAAAAGCCACATGTGAGAGGTATGGTT
GAAATGAGAGGTTGTGTTTGCCAAGTTGTCTTCTGACTGTGGAGAGCTGGTGAGCCTCTTCT
CATCTCCTGGGGCTCCATATTATAGAGCTGACGAATCTCTTTTCTTGCTCCTTGAAGTCTTT
CATTCACTTATTTATTAATTCATTTAACACATCAGGCACCTACTAACTTCTCAGAGTTCAAG
GCTTCCTAGCCTATGATCAGAAAGGACACCTGTGTGCTCATGAGCACCCCTGGGATCAGGGT
GACTGATAGGGTGCTGTCATCACTACTAGATGAAACTCTTTGGAACAAAGGTCTAGGATATA
ATATTTATCCTTCTGCAAATATTCATATGGCATTCGCTGTGTACCAGGCCTTCTGTTGAACA
TTGGCCTGCAGAGCTGAAGAGGACGTGGGTCTTCTCTTAGGAACTCCTAGTCTTGGGAAAAA
GGAATGGGGAAGGGCTGTAATGTGAAGTTAAAAAAAAGTGCTGAGGCCGAACACATCTGGGC
CCTCACTGCAGCCCTATTGCACACACACTGCATCACCTCGAACAAGTTATTCACCCTCTCTG
AATTCATCTATTTGCCCATAAAAATAGAGATGATCCTTTTATATGTTGGCTACCACTTATTA
TGTGCAGCATGCTTGTTATTGTACTAAGTTACTCATTTTTTAGTTTTTCATATAACTCATAT
TTTTGTTGATTTTTATTTTAAAATTCCAAACGAATTAATTAAACTATTTTCCAAAATACGTA
GTATGTGTACATGGTAAAAAACATTTCCAAAAGTTTACCATAAAAACTGTCTCCTTTCCCAT
ATCCTCAATCCTTCAGAATCCCTCTCCAGGGATAATTACCACCACCAGTGTATTCCTTAAGA
GATATTTAAACTTTATACAAAATACGCACACATCATTCTTTTCCACAAATGACAGCATACTA
TGTACATGGTACCTCACCTAGCTTTTTTCACTTACCAGTATATCTTAGAGATTGTTGCATGA
CAGAATATACAGATCTGCCTGTTTTTGTTGTTTTTCCCAAGTTTCCAAGAGCTAGAAACTGT
TTGGTTTTTAAGCAGCTGCTTGGTATTCCATTAATTGGACTTATCGTGCTTTGATCTGTCCC
TAGTGATGGACATTGGGGTTGTTTCCCTTCATTTATATTTGAAGCTTGTTGCAGTGACAGTG
TACACACTTGCTTGAGCATGTGTGTGTCTACGATAAATCCCAATAAGCAATTTAAAGTAATT
TAATTCTTATGCTCTCAACAAACCTAAGAGGTTATTTTTTTAGAGGAGGAAGCTAAGGCTGT
TGTTTTGATGCCTTTTTACCACTGGACTTGGGACTCAACTGTTTGATAGTCATCGAGTCCTT
GACTCTTCTCCCCTGCCAGGATTTGGCCCCTAAGCCCAGGGCCCGAGTCTCCTCCATCTTCA
AGGGAGAGTGGGAACAGCACAGGCCTTGGCCTCAGTCCTGCTCCCCCTGCTTCTAGCTGTGG
GATGGGCCAGGTGCTTCACCCAGCTGTGCCTCCTGTGAGGCACAGTGTGTGCAAAATGGAAA
TGTGAACATGAAGATCAAAAATGTCCCTCAATGACCAGTGCTGTTCCTCAGAGTCACGTGGG
AACTCATAGAAAGCGATATTGGTACTGCTCTTTCCTCTGTAGCATGGTCCAGATGGCTCATA
GCAGGGACCATGATATGCTGGGTGAGCACCCACTGCATGCACCCACTGTGCCAGCACTGAGA
GACTCCTGTGGGAGCCACAGCAATTCTAGGGTCTTCACTGGGGACTCTGAGACAGCAGGGAG
CTAGGATGAGGGCTGCAGAGTGTTCGTCTGCCCTCACTGAGCAGACCCCCTGGATGGCAGGG
AGCAGTCCCAAGCCAGATGGATGCCCATAACCAGCCATTTGGCTCTCAATACATAATATCAC
CACGTATCAGGCAAAACCATCCTGCCCAGAGCATTATCTGAATTTGCATCCCATCTGCAGAA
GATACATTCACCCACTTCTTCCATTCTGTCTTAATCAAAGTCTTTATGTGAATTTTCCCCAT
TGAGAAGACAAGCCCCTTCCTGGCTTAGACTGTACCTGACTGATCTTTTCATGAGCTCCTTG
CCAAGCCAGACCACCCCCAGCTTATATGGAGACTTGGTGCAAATTAGAGATGCCCCTGTGCA
CGTGGCAGCCCTGAGCCCAAGCACCCAGTAAGGCAAAGGGCCTGATTTGGGACCCCTCTGCC
ACTCCACCAGGCAATCAGTTGCTTATTTCTAACTTTCCCTTCCTTCTCCACATTTGTCCCAT
TCCTTCCTCTCATCATGAATATCCCCAGAGGCATTCAGCAGTGCAGTGAATTAAATATAGAA
CTTTTTTTTTTCAGAATTGCAGAACGGATTAGATCAATATTAATCCAAACAGAGCAATGAGC
CTGACAGTTTAGTAAAAGCTCAATAAAGGGTGGCTTACCTCCCCCAAAATAATCTGAAAAGA
AAGCATGTCTTATTTCAGGGGGAAAAAAAAATAAAGTGACCTTTAAAGACCAAATTCCCAGG
ATACCCAGGGTGGAGGTGGAACATGGGAGTCCACAGGCAGCCTGGATGTTTCCAAAGATCCA
AAGGGCTTTTGCTTCCTCACATAATGCAGGAAACAAATTGAACATGTATTAAGTGCTTGCTG
TATGTGACACACTGTGCCAGGTGCTCCCCTTAAAACAGTTCTGTGGGCAGGCATGAGAATGA
ATCCCGATCTTACAGACAAGGAATGTTAGGCTCAGAGGTTTCAAGCTCACCCATCACTCAGC
CAGAGAGGACAGATGCAGGATTCAATCTCGGGAGTGCCCGAGTCCACAGAAGTTCCTGTGCT
GAAGGACCGACCACAGGCACATAAAGAGATGCGAGACAATTTTTACTGGATTTGGCCACCTC
TCGAGGTCGGCTTTGCCAGCTCTTCTCACTGGGGGAAGGGGAGGGAGAAAGTAGCTAGCTCC
AGGGTCCCTAACATAGAACCACCAAGGACTTGACTATTTTTACTCATACAGCAGCTTGTCTG
GGAAGATCATGCTCTGTGACAAGCTGCAGGCACTAAGTAGCAATTTCTGTTTCCCACATATT
AGCTTGAGTCATATAAAACTGACATGGATGTGGCTCAAAAATAGCTGTATGTCAGCCATTTT
ATACCATTTGACTTAAATGTTATTAATTAACGTCACAGCCAGAGATTATTCTCTGAGAAAAG
GGCATTGTAGCCTGAAGCAGAGAAAGCATACACGTTCCCTGGGGTTGAGAACTCATCACAGC
CTGAGACAGCTTAGGTTGTAAAGCCCCGGCCCACTTATCCCAGGAGAGTCTGGGTGAGATGC
AGGCCCCAAAGCAGAGGCTGGGAAGCGAGAAGTGACACACCCTGGCTGGGTGGGCCCTCATC
TTGGTGAGACACCACCTGGGTAAAACCATCATGGAAAGGGTGTAGTGGGGCGTGGAAACTCC
CTCGGTTAAAGCGTGAGCTTTGCTGTAAGTTGTGGTAAGGAGGGAGGCAGTGACAACCAGGA
GGCCTGTTTTGAGGGTTTCTGAGGGACCCATCTGTGGTATCACGAGGAGACGCCCAGAGGAG
CCGTGTGAAAGGGCTGCCTCCCAGCCGGCTCTGGAGTGAATGAGCAGCAAGTCCTGGCTGCG
AAAAGAAGGGGAGTGCAGCCTGCAGAAGTGTCTTCTTTTTTCAATTCCTGCTCAGAAGGAAA
CAGGAGATAAGAATAGTGGGGAAGTCCAAACCAAAGTGAACTATAGGGCTGGTAATCGTAGG
GGGAATTAGTCACCCGGAGACTAGCCCAGCAGACTAACGGAGCCCCATCCTCCATCTTGAAT
CAGTCAGCCCCTCTATGACTGCAGAGTCCTGAATGATGGCAACACCTTCTCTTCACTTAGCG
TTGTAGGATGACCAACAGTCCTGATTTGCCTGGGACTGAGGGGTTCCCAATAGATGGGACTT
TCAGGGCTAAAACCAGGAAAGTCCTGGGCAGCCCAAAACAAGGTAGTCACTCTAGAGTGTAT
GACTCTGTCTGATACCTGCTAAGAAAGAGAAGGACTTGTTGATTATAAGGAGAAGAGGAGGT
GAAATGGTTCTCAAAAAACAAAGATGAGGGCTTCCGGGTGCTGTTCTGCCCAAGGCTCTGGG
TCTGAGGCTTCTCTCTCCAGGCCTAGCTTCATGGAAAAGTAAGGGGCCAGAGGGTGGAAAAG
GTGGAAACAAAGGAAGAGGATGGAGAATTGCTTTGGGGAAGTTTGGACTGGAAGTGTGAATT
ACAGCTGCACCCCCAATTCACCCCATCTCACCCCCCTCCCCCTCCTGCTCATGGTTCTCCCT
TTCTCATCCACACATTGGTCAAACTAGCTAGCTTTTGGAGAGATTTTGGGCAGTAAAAGTAA
AACAGATCTGTCTCAAGCTTCAAAAAGCCTAGAGCTGGCTGGGCGCTGTGGCTCACGCCTGT
AATCCTAGCATTTTGGGAGGCTGAGGCGGAAGGATAATCTGAGGTCAGGAGTTTGAGACCAG
CCTGGCTAACATGATGAAACCCCATCTCTACTAAAAATACAAAAATTAGCCAGGCGTGGTAG
TGCACGCCTATAATCCCAGCTATTTGGGAGGCTGAGGCAGGAGAATCGCTTGAACCCCAGGG
GACAGAGGTTGCAGTGAGCTGAGATCGCACCACTGCACTCCAGCCTGGGTGACACAGCGAGA
CTCCATTTAAAAAAAAAAAAATGCCTAGAGCCAAATGCTCACAGAGCCATTTACTGCATGGC
TTTGGGCAAGTCAAAGGAGTCCGCCTCTCCTGTCAGAAGAGTCTGTTGCAGTCTTCATCACA
AGACTGTTGTGGGGATTAAACAAGATGGCAAGTGGGAAGTTGGGAAATGTAGTGTGCACCCA
ACCAATATTTGTTTCTTCCTGCCTGCCTACATATGAGGCCACACAGAATTCCAACTTTGTTT
CTCTGATAACTAACACAGTTACTTGTTTTTCTTTCTGATCCAG
>CX3CR1-202 utr3: protein_coding
(SEQ ID NO: 9)
AGGGAATCCCAAAGCCTTGTGTCTACAGAGAACCTGGAGTTCCTGAACCTGATGCTGACTAG
TGAGGAAAGATTTTTGTTGTTATTTCTTACAGGCACAAAATGATGGACCCAATGCACACAAA
ACAACCCTAGAGTGTTGTTGAGAATTGTGCTCAAAATTTGAAGAATGAACAAATTGAACTCT
TTGAATGACAAAGAGTAGACATTTCTCTTACTGCAAATGTCATCAGAACTTTTTGGTTTGCA
GATGACAAAAATTCAACTCAGACTAGTTTAGTTAAATGAGGGTGGTGAATATTGTTCATATT
GTGGCACAAGCAAAAGGGTGTCTGAGCCCTCAAAGTGAGGGGAAACCAGGGCCTGAGCCAAG
CTAGAATTCCCTCTCTCTGACTCTCAAATCTTTTAGTCATTATAGATCCCCCAGACTTTACA
TGACACAGCTTTATCACCAGAGAGGGACTGACACCCATGTTTCTCTGGCCCCAAGGGCAAAA
TTCCCAGGGAAGTGCTCTGATAGGCCAAGTTTGTATCAGGTGCCCATCCCTGGAAGGTGCTG
TTATCCATGGGGAAGGGATATATAAGATGGAAGCTTCCAGTCCAATCTCATGGAGAAGCAGA
AATACATATTTCCAAGAAGTTGGATGGGTGGGTACTATTCTGATTACACAAAACAAATGCCA
CACATCACCCTTACCATGTGCCTGATCCAGCCTCTCCCCTGATTACACCAGCCTCGTCTTCA
TTAAGCCCTCTTCCATCATGTCCCCAAACCTGCAAGGGCTCCCCACTGCCTACTGCATCGAG
TCAAAACTCAAATGCTTGGCTTCTCATACGTCCACCATGGGGTCCTACCAATAGATTCCCCA
TTGCCTCCTCCTTCCCAAAGGACTCCACCCATCCTATCAGCCTGTCTCTTCCATATGACCTC
ATGCATCTCCACCTGCTCCCAGGCCAGTAAGGGAAATAGAAAAACCCTGCCCCCAAATAAGA
AGGGATGGATTCCAACCCCAACTCCAGTAGCTTGGGACAAATCAAGCTTCAGTTTCCTGGTC
TGTAGAAGAGGGATAAGGTACCTTTCACATAGAGATCATCCTTTCCAGCATGAGGAACTAGC
CACCAACTCTTGCAGGTCTCAACCCTTTTGTCTGCCTCTTAGACTTCTGCTTTCCACACCTG
GCACTGCTGTGCTGTGCCCAAGTTGTGGTGCTGACAAAGCTTGGAAGAGCCTGCAGGTGCTG
CTGCGTGGCATAGCCCAGACACAGAAGAGGCTGGTTCTTACGATGGCACCCAGTGAGCACTC
CCAAGTCTACAGAGTGATAGCCTTCCGTAACCCAACTCTCCTGGACTGCCTTGAATATCCCC
TCCCAGTCACCTTGTGGCAAGCCCCTGCCCATCTGGGAAAATACCCCATCATTCATGCTACT
GCCAACCTGGGGAGCCAGGGCTATGGGAGCAGCTTTTTTTTCCCCCCTAGAAACGTTTGGAA
CAATCTAAAAGTTTAAAGCTCGAAAACAATTGTAATAATGCTAAAGAAAAAGTCATCCAATC
TAACCACATCAATATTGTCATTCCTGTATTCACCCGTCCAGACCTTGTTCACACTCTCACAT
GTTTAGAGTTGCAATCGTAATGTACAGATGGTTTTATAATCTGATTTGTTTTCCTCTTAACG
TTAGACCACAAATAGTGCTCGCTTTCTATGTAGTTTGGTAATTATCATTTTAGAAGACTCTA
CCAGACTGTGTATTCATTGAAGTCAGATGTGGTAACTGTTAAATTGCTGTGTATCTGATAGC
TCTTTGGCAGTCTATATGTTTGTATAATGAATGAGAGAATAAGTCATGTTCCTTCAAGATCA
TGTACCCCAATTTACTTGCCATTACTCAATTGATAAACATTTAACTTGTTTCCAATGTTTAG
CAAATACATATTTTATAGAACTTC
>CX3CR1-202 utr5: protein_coding
(SEQ ID NO: 10)
GAAATACTCGTCTCTGGTAAAGTCTGAGCAGGACAGGGTGGCTGACTGGCAGATCCAGAGGT
TCCCTTGGCAGTCCACGCCAGGCCTTCACC
>3 dna: chromosome chromosome: GRCh38:3:39263446:39280086:-1
(SEQ ID NO: 11)
ATACTATTGGCTAACTCTGCAAATAAAATGCTCTTAGAGGGAAGGAAAGGGAAATACTCGTC
TCTGGTAAAGTCTGAGCAGGACAGGGTGGCTGACTGGCAGATCCAGAGGTTCCCTTGGCAGT
CCACGCCAGGTAGGTGCACAGGACTAGTTGGGTACCTGTGGGTGGGGTGGAGCAGTGGACAG
CTAATAGGTTAATAATGCCTGTTTGCTTACGTGCAGACAATGGAAACCATTTTCCTGGGGAT
GTTGTAGCCTAAATATGTCCAAGGGGATGGAAGAGTGGGAGGCAAGGGGTGATCAGATCATT
TATAATACACTCAACCTGGTGGAATAGTATTAGAAGCATTAGTAATTACATTTTAGAGACAT
GGAGAAAAGCTCATGATTTTAAACTAACTGAAAAAAGCATGAAAAATTGCATCTGGATGCTG
TTGAGGAATTATTGCTAATTTTTTGAGATGAGAAATTGTATTAAAGTCTTTTTCAAAAAAGA
GTCCTTAACTTTTAGAGATGCACACAAGTGTTTATGGGTGAAATTAAATAATTCAGTAGAGA
CATTAAGTGGGAAATAGAGGAAATATTGACCATGGGTTGCTAATAGTTGAAGCCAGGTGTTG
GGTATAAGGAGGTTCTCGCTGCTTTTATTTGAAAAGTTGCTTTTTATTTGAAAATTTAATAA
TAAAGAGTTTTTAAATTTGTATCTGTATTTTAATATAAATATAAATGCACTTAATATAAATA
TAAAATATGTATAACATTTAGATAGAGAAAAGCTAAAAGATTACTGTGGTTGATTCTAGGAA
ACTGCATTGCAGATAGTTTCATGGGTTTTTTTTTCCTTTTTCTTCCACTTTTTGTACCATCT
ATGGGTTTTTGTTTGTTTTTTGTTTTTTGGGTTTTCTTTCTTGTTTGTGTTTTGTTTTTTGA
GACGGAGTTTTGCTCTTGTTGCCTAAGCTGGAGTGCAATGGCACAGTCTCGGCTCACTGCAA
CCTCTGCCTCCTGGGTTCAAGTGATTCTCCTGCCTCAGCCTCCCAAGTAGCTGGGATTATAG
GCATGTACCACCGCCCGGCTAATTTTGTATTTTTAGTAGAGGCGGGGTTTCTCCATTAATAA
ATTCCTGGCACAAATTTAGTGTTCAATTTTGATATATGTTGTTATAACCATTGTGAGGATAC
TCAGGCTCAGGTTTGTGTGGGTGGAAAACATGGTCTTCAGAAAGAAATTATGAGTGCAAGAC
AGGAGGAAATCCATCAGAGGCCCCAGCTGAGGACTGACCACGGCTTGTTATTTCTCTTGCCT
TGCCTCTGGCAATCACAGCCTCACAGAGCCTGCAATCCTTGCTTTGTGAGTTTATAGCTCAG
TCCAGAGAATGGCTAAGAAAGTTTAGGATTCTTTCAACACCCACTCCACAAAAAAAAAAAAA
AAAAAGAAAAGAAAAAAAAATTAATTTTTGAAATACTTGAGGTAGAAAACTTGAGGCAGAAA
AAAATTGAGCCAAAAAAAAAGGAAAATTGAACCACGTGAAAGCAGGCAAGAAAGCTTGCATT
GCTCAGGGCATCCCAGGCCCAGAGGGCGCTTTGGAGGGAGCTGGGTTTCCTGAGAGGAGGCA
GGGTGGGTGACGGACCTGTGCTGGAGAGCCTTGAGGACCACTGTGGGTTGGGAATGGGGGCA
GTGGATTGGGGTTCAAAACCCCTGGGAATGAGAAATGGGCTCAGGAAGGCTAGGGTGGATTC
TTTCATCTTCCTCTTTGCTTGGCTTTATTTTCACAAAGGAAGGCAGGGCAGGAAATAGTCTC
AGCCCAACTTCAGTGTGGTTCTTCTTAGTGCTCAGGCTTACCTGGCACTTGCCACACCTCTG
GGATGGGAGCACCTACTATCCATCAGCCACGTGCCAGTCTCCACAAAGTCTGCTCCTGAACC
CTGCTCCTCAGCTGGCCCCACTTCACAGATGGGGACATAGGCAGCTTGGCTTTGGAATGAAG
GAATGAAGTCAGGAATGAAGTCCTGGCTCTGCACTTGGTGACTGTGCACTGGGCTTGCTAAG
TCTGTTTCCTGCTTTTAAAATGGAGATTGTCCATCAGCCTTTGAAGCCATGTAATGGGTATG
TGTCAACTTTCTGCAAGGATTAAAGGCATGGTATAGGAAGTCCCAAACACACTGCCTGACCC
ATCTTTGATGCTCAAGAAACGATATATGTTGTTGTCATGAGGAAACTGAGCCTCAGAAAGTT
TGGATACCTGAAAAACACTGACTACTATTGAATGAGGTTGTGAAGAATCCAGAGCTGTAGGG
GCAGGAAAGCAAAGAACGTATTAGAGCTGACCCAGTCAGGACGATCGTCTATCCCCTTCCTC
ACCCCACCCCATCCCAGGAGGAAGCCTGCCCGGCCCTAGGCAGCTATGGCACAGTGGCAATG
TCAGGTATGGTTCTCCCTAGCCAGAGACCCTAGCCTCAAAAAACCTCCTTCTTGGGATCCAG
GCATCCAACTGCTCCTCCCCAGCCCCAGCCTCTGACCCAGTATCCTGAGTCCAGAGACGTTT
GGAACCAGCACCTGTAATGGAGGAGCTGAACAAGGAGGGGAACTTCTGCTGCTCCACAGCAG
GTCACGGTCATAGGAGGGAGTGGAACCAGAATGGCAGAATCCAGATCTTGGCTGCCTTTCCC
AAGGACTTGTTCTGATTCCTAGCAGCACAGCCCAGGCATTCCGAGAAGTTGGGCTCTCTGGC
ATCACTCACTCTGCCCAGAAGAGCCAGGGGAAAGTTGGGGCTTCTAGCTGAACCTTGATCCC
ACCTGCCCTCTTGAGGGGCTCAGAATCTGCTGGCTGCTTCACAGGTGGGATTCTCACGGCAC
GCTGGCCACAGCTGATGCTTCGACCCCCTCATCTTGTTTGGCCAAAGTGCAGCTTTTTAGCT
TGTGAGTAAGGAAGAAAAGCTGTATCATATGTCTTTAAACATCTTCCTAGACCACCTTTGTT
TTCCCCTTAAAGTGTGCTTAAGGAGAAAATGGAAAGTCTCTTTTCAGTGTGTTGTATTTTGT
TTATTGCAAAATACAACACACTGAAAGAACAATGTCTGGGTTAGCAAAGTATTAGATTTAAT
TTCCCACATTACTACCATCCAGGCTGAGAAAGAGAACCCCAGAAGCCTCTCTCATGTCCCTT
CCTGACCACAGCCTCTTCCTCTCCCACAAAGAACCACAAGTCTGACTTCTATGGTGATCACT
TCCTTGCTTTTCCCTATAGTTTGACTGAATCTAAATATCATCCCTAAACAATATAGCTTAGC
TTTGCCTATTTTTGATCTTCATATTTGGATTCATACTTTTTGGTTTGGCTCAAGACTAAACT
TTTAAAATGCATCCACGTTGTAGGAGATGTACATGCACAAAAATTTTCACGGCAACATTGTC
CAAACTAGCAACAAACTGAAAGCCACCTATGTGTGCATTAGCAGTAGAAATGGCAATAAATC
ATGGACTAGTCACACAGTGGAATCCTACGCAGCAAGGAGAATTAGCAGTCTACAGCCAAACC
AACAGCATGGGTGAGATGCTTCCAGAAATACTGAGATGAATTTAGAGATAAAGGCACAATTG
GTCCATATCTACAGGGCACTTAATCCAGTGCATCTCCACTAGACAAAATTTGTATTTCCATG
AGCAAGAGTCATTTGCACTGCTATCTGCTATAACCTACGGTGCTGTAAAATTAGCTCAGGCA
ATAAAAGGGGGAGGTAGCCCCAAAGAGTATGCCAGAAAGGACTCCCAGTAATCTTCAGTGTG
TGTCTATTTCAAAGTCTTTCATCATTTTTCCTTGTAGTTACTTAGTGTGGAATTTCAGACCC
CTCTCTATGTCCCTCTTCTCCCTTTTCAGCCTTGGCTTTCTCTGCATCCTTCCCCGAAGCCC
TGCACTCAGCCTAAACTGACTTTCCTGAACACTCTGGGAGCATGTGGGCTCCTCCCAACTCC
ACCATCTTCACTGCCTTCTGTTCCTATTTCCCTCTGCCTGGCCTTGGCCCCAGGAAACCTTC
CTATGCTCAGACCCTCTGTGCCTTTGTCTTCAAAGCCCACCCACTATTCACTGCCACCTCCA
TCTTGGTCAACCTGGAAGACTTTCCCCATCCTTAAAACATCAGCTCAAACGGCAGATTTTTT
TTTTTTTTTTTGTGATATCCTCTTGATCACCCTCCCCCAGTTTTTAAGGAGACAGATATAGA
GGTTCAATCCTTGGGCTTCCCATGACTTTTCTTATATTCTGTTACTGAGCAATAATAAACAC
TTCTAGGAACTGTATCTTAAACACTTTTGCCTCTACAGAATCTAGCACAGTGCCTAGTATTG
GTGAAATATAATTAACAAAGTCTTCTTTCAACACAGAGATTCTCTCCACAAAAGGAGTAGAG
AAAGAACAGTTTTATTATGGAATAAGCAGTAAACCAAAATATGCAGAGCATTATAGGCCATC
TACTAAGAGGTTGCAAGAACAGAAAGAAATCTCCCCCTTTTGTATAGCCAAGTAGATACAAC
CTGTTACATACATGTTATCAAGGCAAACAATAACTGTTCCTCAAGTAAGAGGTCTTGCCAGC
ACCGTTTGCCGTACATGGTTCACTCTAAATTTACCTGGTAATTAGGGTAACCACTTGTGTTA
GCTAACTGGCTCTACCCAGAGGAAAATCAAATTTATCTTTAAGACAAGGGGTAATTTTGCAG
CACTGAGCAAGGCTCTTCAGTTAGGCTCATACCTTCCCACAGAAACTAAGAGATAGAAGCAC
TATCTCCCTTAGGTTTGTTTACATTTCAAAGAGATGACGCCCAGGTCCTTGGGAAAGACTAG
CTTAGCTCATAAAGCTGACAAAAAGCCTATCTAGTTTCAAAAGGATTTACACATGTTCAAAG
AGAGGAGAAAGTATGTAAAAGTTTTCTAGGTGGGCACGGTGGCTCACACCTGTATTCCCAGC
ACTTTGAGAGGCTGAGGCGGGTGGATCACCTGAAGTCAGGAATTTGAGATCAGCCTGGCCAA
CATGGTGAAACCTTGTCTCTACTAAAAATACAAAAATTAGCTGGGCATGGTGGTGGGCGCCT
GTAATCCCAGCTACTTGGGAGGCTGAGGCAGGAGAATCACTTGAACCCAGGAGGTGGAGGTG
GCAGTGAGCCAAGATGGCACCATTGCACTCCAGCCTGGGCGATGAGAGTGAAACTCCATGTC
AAAAAAAAAAAAAGTTTTCTAAAGTAAATGCTCTGAGAAAAAAACAGAAGGTGAGGAGATCT
CTGTCCTTATTTTTAACAAGAATAATTAAATGTTTTTATTTTTAATTTCTACTTAGACTAGA
ATATGGTACAGATGCTCAAAAATATTGACTTAATTAATAATTAAACAATTAACAAGTCTATC
TCTTTCCTAATTATTAAAAAAATTATGAAGTATTTCCCTTCAAAATTTCACAGTTTACTTTG
ACATAAATAAATAAATACCATAAAGTATTTTGAAGAGGAAAGGGAAGGGTGGGGCTTCAAAC
ACAAAATAATGGCAGGTTTTGTAAAGGGGCAATTCACTAAACCCAATAGGATTCTCCTGTTG
CTATTTTTTTTTTTTTTTTTTTTTTGGTGGTGGTTGGAGGGAAGGGTTGGGTGTGCATTTGG
CCTGTGTGAAAAACTGGTGGGTAGACAATGCCATGCTGTTCTAGATGGGCTCATTGCTATAG
GAATAGATAGTCACTGAATTTACTTGCATAGGGGATGGGGTATAAAATGTTTCCTTATAGCT
CCTTAACATGGAATTCAGCACCCCTCTCCCCATCAGCCTTAGCTCTCTCTGAATCCCAAGGG
ATATGTGTTATAAAAATAGCTGCCATCATAAATGAGAAAACCATCAGGACCTGGTAAAACTT
GGCTTCCCAAACAGTCAGGGGTCTGGGCATGGCAGCCGACTGAGAACCTTTCTATCTAGTAC
AAGGTAAACAAGATTTGCCAAGACTTGATCAACTGATCCAGCTCCCTATCTCCAAACAGAAC
CTCATCTGAATGCTCACAGGCTGTGCCCCAGAAGTGTAGAAGTGTCATGTTCCCTGGGCAGG
CTGCTGGGCAGCCTCTCTGTTGACAATAGACCACACTTTTGCTGACCTAGGACTCATGTTGC
TCTTTAAGACTGCTTCCTTGGCCAGGCATGATGGCTCACACCTGTAATCCTGGCACTTTGAG
AGGCCCAGGCAGGAGGATCTCTTGAGGCCAGATGTTCAAGACCAGCCTGGTCAACATAGTAA
GACCCCATATCTACCAAAAATAGCTGGGCATGGTGGTGCACACCTATACTCCCAGCTACTTA
GGAGACTGAGGTGGGAGGATTGCTACATCCCAGGAGTTCAAGGCTGTAGTGAGCTATGATCA
TGCCACTGCACTCCAGCCTGGGCAACAGAGCGAGATCCTGTCTCAAACAAACAGTTTCCTTC
TGTTTGATTCTTGCTGAAAAATTGAGCATGCCAGAGCTAGCAAGGCTCTTAGAGGTGGTCTG
GTCCAATGCTTTAATTTCATACATCAAGAAACTGAAGCAGAGCAGAGTGACCTGCCCAGGGT
CTCTCAGCCATTCATGCTCAGAAATGTATGGGCTCCTGTGAAACATGTGGCTCTTAAAAGCA
CTATCATATATTTGAAGGCAGAAAATAGGCTAAACCTTCAGCCTTCAGACTTTTCCTCTCCA
GAGAAAATGACCCCAGTTTCCTCACTATGGTTGCTGGGAGCTAGATTCCTGGGGATCTGGCA
GTGTGGACCACCTAGTGGTGGCTAGAGGAGCAAATAATATCCCGCATTCCATTTTCCACTCA
CCAATCCCTGAGGGGCAGCCTGCTGGGTTATGAGCCCACAGGGGGAGAACCCCAACGAATTC
AGAGATGCATCATGGACCAGTTTCTCTTAAGGGGCCTGGGTCTACTATTTTCAGTTCTACTT
CGAGAGAAGTGGCCTGCAATATCCTGCAGATTTCCCCTCCAGGGAGAAAAGCATTGTGCGGT
GCAAGGAGCACAGGCTTTGTGGAAAGAGGCATCTGGGGTTGAGATCCTGGCTCTTTTGCTTC
CTTGAACAAGTTAACCAAATCTCTGGGCCTCTGCTGGTTAATTTATACCATGGGGATCATCA
TTTCTCCAGTGGGGTTGTGAAGAGAATGTGGTGGGATCTTGTGTGTGGAGCATGACACTTAG
CAGGCATCCGGGAATGGCAGCCTCCTCCCTTTCTAAACTGGGGCTTTCTGAGGGTGACTTCA
GATTCCACAATGTCAACAGCACAATGGCATCCTCATAAGGAAAGTTTGGGTTGGGGCTCCTC
AAGCAATTCTCTACTCTCATTTGGTACAAAGAAAAAAATTAAGCCTCACAATTTTCTTGGCA
CCAGACTGAACCTCAAACCCAGTCTTCACTTTTACTAAAAAGCCATAAACAGAGACCAGGAG
GGTAAAAACTACCAGAAGATACACTGGATTTAGAAAACAGTAAGCTGGATGTGAAGCCAAAA
GAGAGGGAGAAAGCAACATAAAAGAATGTCAGATTGAAGAAGAAATGGATTCTGGCCACCTA
AAGAAGAAGGGAAGTATAGAAAAGGGAAGCTGATTGGAAACAATGGGTAATGATGAGTGTCC
CTCCCCTAAAAAGTTAAAAAATAAATGAGCAGGCATGAGAAAAGGAAGTAGGCCAGAGGAAG
TAGACCAGGATCAGCCACGGATGTGAGTGGGGAATTTTACAAATTTCTACTTGGGTGTGAAA
CATGTGTACACTCAAAGCTCTGTCTAGGTTCACCGAAACTCTCCCAGCTCAAGGCCATTTAG
TAAAACCTTGGCTTAACTGAATAGTGGGTAAGGAGGCCTGAGGATGGCCCAGAGAAGGTTTA
AATTCTTACTGGCTGCTGCACTAAGTTGTAAAGTGCAGCCTTAGTTGATGATGCCTTTATTA
GTGCAACATCCCAGAATGCATGCGTCTTACAACCCTTGTGGATATGAAACCCAGCTGGGTAG
ACACTGCAAAGTCTTCTCATTTGATTCTTCCACTTGGTTTCTAGTGTCCCTCAGGGACAAAG
GAAAGCCATGGTCCAGTCTAAGATGAAAACCAATTAGACCTCTGGCAGGCCTTTGACCACCC
TGAGGGCCACCCCCAAGGCACGGCCACACTCGCATCTGCTGCCAGGAGGCCCTATCTTAGGC
TCAGCTCCCAAACCTTGAACATCTTGGAGGATCCACAAATAGGAGGTACACTCAGCTCAGGC
TCAGTCTTCCTTAAAAAGCGCCTTGCTAAAGCTATCAAGTTCACTGGAATACTTCTGCGAAG
GACACAGCTTCAGCGATGTCAGATTTTTTATGTAAATGGTGCCTTCACATGCTCTGGGGCAT
TTGGCTACCAAGGGCGGTTTGAACTAGCTCCAGCACACAGAATACAAGTCTCTTCAGAACCA
GGCAAAACCCTATGTTGCCCAATGACTCCTGCTGTTTCTTGAGATTTGCACAGAAGACAGAG
CTGCAATTACCCACGCTGATATCTATTTCATGACCACATATGTTCAAAAGCCACATGTGAGA
GGTATGGTTGAAATGAGAGGTTGTGTTTGCCAAGTTGTCTTCTGACTGTGGAGAGCTGGTGA
GCCTCTTCTCATCTCCTGGGGCTCCATATTATAGAGCTGACGAATCTCTTTTCTTGCTCCTT
GAAGTCTTTCATTCACTTATTTATTAATTCATTTAACACATCAGGCACCTACTAACTTCTCA
GAGTTCAAGGCTTCCTAGCCTATGATCAGAAAGGACACCTGTGTGCTCATGAGCACCCCTGG
GATCAGGGTGACTGATAGGGTGCTGTCATCACTACTAGATGAAACTCTTTGGAACAAAGGTC
TAGGATATAATATTTATCCTTCTGCAAATATTCATATGGCATTCGCTGTGTACCAGGCCTTC
TGTTGAACATTGGCCTGCAGAGCTGAAGAGGACGTGGGTCTTCTCTTAGGAACTCCTAGTCT
TGGGAAAAAGGAATGGGGAAGGGCTGTAATGTGAAGTTAAAAAAAAGTGCTGAGGCCGAACA
CATCTGGGCCCTCACTGCAGCCCTATTGCACACACACTGCATCACCTCGAACAAGTTATTCA
CCCTCTCTGAATTCATCTATTTGCCCATAAAAATAGAGATGATCCTTTTATATGTTGGCTAC
CACTTATTATGTGCAGCATGCTTGTTATTGTACTAAGTTACTCATTTTTTAGTTTTTCATAT
AACTCATATTTTTGTTGATTTTTATTTTAAAATTCCAAACGAATTAATTAAACTATTTTCCA
AAATACGTAGTATGTGTACATGGTAAAAAACATTTCCAAAAGTTTACCATAAAAACTGTCTC
CTTTCCCATATCCTCAATCCTTCAGAATCCCTCTCCAGGGATAATTACCACCACCAGTGTAT
TCCTTAAGAGATATTTAAACTTTATACAAAATACGCACACATCATTCTTTTCCACAAATGAC
AGCATACTATGTACATGGTACCTCACCTAGCTTTTTTCACTTACCAGTATATCTTAGAGATT
GTTGCATGACAGAATATACAGATCTGCCTGTTTTTGTTGTTTTTCCCAAGTTTCCAAGAGCT
AGAAACTGTTTGGTTTTTAAGCAGCTGCTTGGTATTCCATTAATTGGACTTATCGTGCTTTG
ATCTGTCCCTAGTGATGGACATTGGGGTTGTTTCCCTTCATTTATATTTGAAGCTTGTTGCA
GTGACAGTGTACACACTTGCTTGAGCATGTGTGTGTCTACGATAAATCCCAATAAGCAATTT
AAAGTAATTTAATTCTTATGCTCTCAACAAACCTAAGAGGTTATTTTTTTAGAGGAGGAAGC
TAAGGCTGTTGTTTTGATGCCTTTTTACCACTGGACTTGGGACTCAACTGTTTGATAGTCAT
CGAGTCCTTGACTCTTCTCCCCTGCCAGGATTTGGCCCCTAAGCCCAGGGCCCGAGTCTCCT
CCATCTTCAAGGGAGAGTGGGAACAGCACAGGCCTTGGCCTCAGTCCTGCTCCCCCTGCTTC
TAGCTGTGGGATGGGCCAGGTGCTTCACCCAGCTGTGCCTCCTGTGAGGCACAGTGTGTGCA
AAATGGAAATGTGAACATGAAGATCAAAAATGTCCCTCAATGACCAGTGCTGTTCCTCAGAG
TCACGTGGGAACTCATAGAAAGCGATATTGGTACTGCTCTTTCCTCTGTAGCATGGTCCAGA
TGGCTCATAGCAGGGACCATGATATGCTGGGTGAGCACCCACTGCATGCACCCACTGTGCCA
GCACTGAGAGACTCCTGTGGGAGCCACAGCAATTCTAGGGTCTTCACTGGGGACTCTGAGAC
AGCAGGGAGCTAGGATGAGGGCTGCAGAGTGTTCGTCTGCCCTCACTGAGCAGACCCCCTGG
ATGGCAGGGAGCAGTCCCAAGCCAGATGGATGCCCATAACCAGCCATTTGGCTCTCAATACA
TAATATCACCACGTATCAGGCAAAACCATCCTGCCCAGAGCATTATCTGAATTTGCATCCCA
TCTGCAGAAGATACATTCACCCACTTCTTCCATTCTGTCTTAATCAAAGTCTTTATGTGAAT
TTTCCCCATTGAGAAGACAAGCCCCTTCCTGGCTTAGACTGTACCTGACTGATCTTTTCATG
AGCTCCTTGCCAAGCCAGACCACCCCCAGCTTATATGGAGACTTGGTGCAAATTAGAGATGC
CCCTGTGCACGTGGCAGCCCTGAGCCCAAGCACCCAGTAAGGCAAAGGGCCTGATTTGGGAC
CCCTCTGCCACTCCACCAGGCAATCAGTTGCTTATTTCTAACTTTCCCTTCCTTCTCCACAT
TTGTCCCATTCCTTCCTCTCATCATGAATATCCCCAGAGGCATTCAGCAGTGCAGTGAATTA
AATATAGAACTTTTTTTTTTCAGAATTGCAGAACGGATTAGATCAATATTAATCCAAACAGA
GCAATGAGCCTGACAGTTTAGTAAAAGCTCAATAAAGGGTGGCTTACCTCCCCCAAAATAAT
CTGAAAAGAAAGCATGTCTTATTTCAGGGGGAAAAAAAAATAAAGTGACCTTTAAAGACCAA
ATTCCCAGGATACCCAGGGTGGAGGTGGAACATGGGAGTCCACAGGCAGCCTGGATGTTTCC
AAAGATCCAAAGGGCTTTTGCTTCCTCACATAATGCAGGAAACAAATTGAACATGTATTAAG
TGCTTGCTGTATGTGACACACTGTGCCAGGTGCTCCCCTTAAAACAGTTCTGTGGGCAGGCA
TGAGAATGAATCCCGATCTTACAGACAAGGAATGTTAGGCTCAGAGGTTTCAAGCTCACCCA
TCACTCAGCCAGAGAGGACAGATGCAGGATTCAATCTCGGGAGTGCCCGAGTCCACAGAAGT
TCCTGTGCTGAAGGACCGACCACAGGCACATAAAGAGATGCGAGACAATTTTTACTGGATTT
GGCCACCTCTCGAGGTCGGCTTTGCCAGCTCTTCTCACTGGGGGAAGGGGAGGGAGAAAGTA
GCTAGCTCCAGGGTCCCTAACATAGAACCACCAAGGACTTGACTATTTTTACTCATACAGCA
GCTTGTCTGGGAAGATCATGCTCTGTGACAAGCTGCAGGCACTAAGTAGCAATTTCTGTTTC
CCACATATTAGCTTGAGTCATATAAAACTGACATGGATGTGGCTCAAAAATAGCTGTATGTC
AGCCATTTTATACCATTTGACTTAAATGTTATTAATTAACGTCACAGCCAGAGATTATTCTC
TGAGAAAAGGGCATTGTAGCCTGAAGCAGAGAAAGCATACACGTTCCCTGGGGTTGAGAACT
CATCACAGCCTGAGACAGCTTAGGTTGTAAAGCCCCGGCCCACTTATCCCAGGAGAGTCTGG
GTGAGATGCAGGCCCCAAAGCAGAGGCTGGGAAGCGAGAAGTGACACACCCTGGCTGGGTGG
GCCCTCATCTTGGTGAGACACCACCTGGGTAAAACCATCATGGAAAGGGTGTAGTGGGGCGT
GGAAACTCCCTCGGTTAAAGCGTGAGCTTTGCTGTAAGTTGTGGTAAGGAGGGAGGCAGTGA
CAACCAGGAGGCCTGTTTTGAGGGTTTCTGAGGGACCCATCTGTGGTATCACGAGGAGACGC
CCAGAGGAGCCGTGTGAAAGGGCTGCCTCCCAGCCGGCTCTGGAGTGAATGAGCAGCAAGTC
CTGGCTGCGAAAAGAAGGGGAGTGCAGCCTGCAGAAGTGTCTTCTTTTTTCAATTCCTGCTC
AGAAGGAAACAGGAGATAAGAATAGTGGGGAAGTCCAAACCAAAGTGAACTATAGGGCTGGT
AATCGTAGGGGGAATTAGTCACCCGGAGACTAGCCCAGCAGACTAACGGAGCCCCATCCTCC
ATCTTGAATCAGTCAGCCCCTCTATGACTGCAGAGTCCTGAATGATGGCAACACCTTCTCTT
CACTTAGCGTTGTAGGATGACCAACAGTCCTGATTTGCCTGGGACTGAGGGGTTCCCAATAG
ATGGGACTTTCAGGGCTAAAACCAGGAAAGTCCTGGGCAGCCCAAAACAAGGTAGTCACTCT
AGAGTGTATGACTCTGTCTGATACCTGCTAAGAAAGAGAAGGACTTGTTGATTATAAGGAGA
AGAGGAGGTGAAATGGTTCTCAAAAAACAAAGATGAGGGCTTCCGGGTGCTGTTCTGCCCAA
GGCTCTGGGTCTGAGGCTTCTCTCTCCAGGCCTAGCTTCATGGAAAAGTAAGGGGCCAGAGG
GTGGAAAAGGTGGAAACAAAGGAAGAGGATGGAGAATTGCTTTGGGGAAGTTTGGACTGGAA
GTGTGAATTACAGCTGCACCCCCAATTCACCCCATCTCACCCCCCTCCCCCTCCTGCTCATG
GTTCTCCCTTTCTCATCCACACATTGGTCAAACTAGCTAGCTTTTGGAGAGATTTTGGGCAG
TAAAAGTAAAACAGATCTGTCTCAAGCTTCAAAAAGCCTAGAGCTGGCTGGGCGCTGTGGCT
CACGCCTGTAATCCTAGCATTTTGGGAGGCTGAGGCGGAAGGATAATCTGAGGTCAGGAGTT
TGAGACCAGCCTGGCTAACATGATGAAACCCCATCTCTACTAAAAATACAAAAATTAGCCAG
GCGTGGTAGTGCACGCCTATAATCCCAGCTATTTGGGAGGCTGAGGCAGGAGAATCGCTTGA
ACCCCAGGGGACAGAGGTTGCAGTGAGCTGAGATCGCACCACTGCACTCCAGCCTGGGTGAC
ACAGCGAGACTCCATTTAAAAAAAAAAAAATGCCTAGAGCCAAATGCTCACAGAGCCATTTA
CTGCATGGCTTTGGGCAAGTCAAAGGAGTCCGCCTCTCCTGTCAGAAGAGTCTGTTGCAGTC
TTCATCACAAGACTGTTGTGGGGATTAAACAAGATGGCAAGTGGGAAGTTGGGAAATGTAGT
GTGCACCCAACCAATATTTGTTTCTTCCTGCCTGCCTACATATGAGGCCACACAGAATTCCA
ACTTTGTTTCTCTGATAACTAACACAGTTACTTGTTTTTCTTTCTGATCCAGGCCTTCACCA
TGGATCAGTTCCCTGAATCAGTGACAGAAAACTTTGAGTACGATGATTTGGCTGAGGCCTGT
TATATTGGGGACATCGTGGTCTTTGGGACTGTGTTCCTGTCCATATTCTACTCCGTCATCTT
TGCCATTGGCCTGGTGGGAAATTTGTTGGTAGTGTTTGCCCTCACCAACAGCAAGAAGCCCA
AGAGTGTCACCGACATTTACCTCCTGAACCTGGCCTTGTCTGATCTGCTGTTTGTAGCCACT
TTGCCCTTCTGGACTCACTATTTGATAAATGAAAAGGGCCTCCACAATGCCATGTGCAAATT
CACTACCGCCTTCTTCTTCATCGGCTTTTTTGGAAGCATATTCTTCATCACCGTCATCAGCA
TTGATAGGTACCTGGCCATCGTCCTGGCCGCCAACTCCATGAACAACCGGACCGTGCAGCAT
GGCGTCACCATCAGCCTAGGCGTCTGGGCAGCAGCCATTTTGGTGGCAGCACCCCAGTTCAT
GTTCACAAAGCAGAAAGAAAATGAATGCCTTGGTGACTACCCCGAGGTCCTCCAGGAAATCT
GGCCCGTGCTCCGCAATGTGGAAACAAATTTTCTTGGCTTCCTACTCCCCCTGCTCATTATG
AGTTATTGCTACTTCAGAATCATCCAGACGCTGTTTTCCTGCAAGAACCACAAGAAAGCCAA
AGCCATTAAACTGATCCTTCTGGTGGTCATCGTGTTTTTCCTCTTCTGGACACCCTACAACG
TTATGATTTTCCTGGAGACGCTTAAGCTCTATGACTTCTTTCCCAGTTGTGACATGAGGAAG
GATCTGAGGCTGGCCCTCAGTGTGACTGAGACGGTTGCATTTAGCCATTGTTGCCTGAATCC
TCTCATCTATGCATTTGCTGGGGAGAAGTTCAGAAGATACCTTTACCACCTGTATGGGAAAT
GCCTGGCTGTCCTGTGTGGGCGCTCAGTCCACGTTGATTTCTCCTCATCTGAATCACAAAGG
AGCAGGCATGGAAGTGTTCTGAGCAGCAATTTTACTTACCACACGAGTGATGGAGATGCATT
GCTCCTTCTCTGAAGGGAATCCCAAAGCCTTGTGTCTACAGAGAACCTGGAGTTCCTGAACC
TGATGCTGACTAGTGAGGAAAGATTTTTGTTGTTATTTCTTACAGGCACAAAATGATGGACC
CAATGCACACAAAACAACCCTAGAGTGTTGTTGAGAATTGTGCTCAAAATTTGAAGAATGAA
CAAATTGAACTCTTTGAATGACAAAGAGTAGACATTTCTCTTACTGCAAATGTCATCAGAAC
TTTTTGGTTTGCAGATGACAAAAATTCAACTCAGACTAGTTTAGTTAAATGAGGGTGGTGAA
TATTGTTCATATTGTGGCACAAGCAAAAGGGTGTCTGAGCCCTCAAAGTGAGGGGAAACCAG
GGCCTGAGCCAAGCTAGAATTCCCTCTCTCTGACTCTCAAATCTTTTAGTCATTATAGATCC
CCCAGACTTTACATGACACAGCTTTATCACCAGAGAGGGACTGACACCCATGTTTCTCTGGC
CCCAAGGGCAAAATTCCCAGGGAAGTGCTCTGATAGGCCAAGTTTGTATCAGGTGCCCATCC
CTGGAAGGTGCTGTTATCCATGGGGAAGGGATATATAAGATGGAAGCTTCCAGTCCAATCTC
ATGGAGAAGCAGAAATACATATTTCCAAGAAGTTGGATGGGTGGGTACTATTCTGATTACAC
AAAACAAATGCCACACATCACCCTTACCATGTGCCTGATCCAGCCTCTCCCCTGATTACACC
AGCCTCGTCTTCATTAAGCCCTCTTCCATCATGTCCCCAAACCTGCAAGGGCTCCCCACTGC
CTACTGCATCGAGTCAAAACTCAAATGCTTGGCTTCTCATACGTCCACCATGGGGTCCTACC
AATAGATTCCCCATTGCCTCCTCCTTCCCAAAGGACTCCACCCATCCTATCAGCCTGTCTCT
TCCATATGACCTCATGCATCTCCACCTGCTCCCAGGCCAGTAAGGGAAATAGAAAAACCCTG
CCCCCAAATAAGAAGGGATGGATTCCAACCCCAACTCCAGTAGCTTGGGACAAATCAAGCTT
CAGTTTCCTGGTCTGTAGAAGAGGGATAAGGTACCTTTCACATAGAGATCATCCTTTCCAGC
ATGAGGAACTAGCCACCAACTCTTGCAGGTCTCAACCCTTTTGTCTGCCTCTTAGACTTCTG
CTTTCCACACCTGGCACTGCTGTGCTGTGCCCAAGTTGTGGTGCTGACAAAGCTTGGAAGAG
CCTGCAGGTGCTGCTGCGTGGCATAGCCCAGACACAGAAGAGGCTGGTTCTTACGATGGCAC
CCAGTGAGCACTCCCAAGTCTACAGAGTGATAGCCTTCCGTAACCCAACTCTCCTGGACTGC
CTTGAATATCCCCTCCCAGTCACCTTGTGGCAAGCCCCTGCCCATCTGGGAAAATACCCCAT
CATTCATGCTACTGCCAACCTGGGGAGCCAGGGCTATGGGAGCAGCTTTTTTTTCCCCCCTA
GAAACGTTTGGAACAATCTAAAAGTTTAAAGCTCGAAAACAATTGTAATAATGCTAAAGAAA
AAGTCATCCAATCTAACCACATCAATATTGTCATTCCTGTATTCACCCGTCCAGACCTTGTT
CACACTCTCACATGTTTAGAGTTGCAATCGTAATGTACAGATGGTTTTATAATCTGATTTGT
TTTCCTCTTAACGTTAGACCACAAATAGTGCTCGCTTTCTATGTAGTTTGGTAATTATCATT
TTAGAAGACTCTACCAGACTGTGTATTCATTGAAGTCAGATGTGGTAACTGTTAAATTGCTG
TGTATCTGATAGCTCTTTGGCAGTCTATATGTTTGTATAATGAATGAGAGAATAAGTCATGT
TCCTTCAAGATCATGTACCCCAATTTACTTGCCATTACTCAATTGATAAACATTTAACTTGT
TTCCAATGTTTAGCAAATACATATTTTATAGAACTTCCATCTGTGTAATCTTCTTTCTCCTA
TTCAATTATTTCCTGTGGTTAAATT

The term “haploinsufficient” refers to a condition where one copy of a gene is inactivated or deleted and the remaining copy of the gene is inadequate to produce sufficient quantities of a gene product to preserve normal function.

As used herein, the terms “determining,” “assessing,” “assaying,” “measuring,” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In one embodiment, the disease is a metabolic or neurological disease or disorder. In some embodiments, the disease treated or ameliorated is one or more of lysosomal disorders (e.g., metachromatic leukodystrophy (MLD), Krabbe disease or globoid cell leukodystrophy (GLD), mucopolysarcharidosis type I (MPS I), mucopolysarcharidosis type II (MPS II), mucopolysarcharidosis type IIIA and IIIB (MPS IIIA and B), GM1 gangliosidosis (GM1)), peroxisomal diseases (e.g., X-linked adrenoleukodystrophy (X-ALD), adrenomyeloneuropathy (AMN)), microgliopathies (e.g., due to CSF1R receptor mutations), neuroinflammatory diseases (e.g., multiple sclerosis (MS)), neurodegenerative disorders (e.g., dementias: Alzheimer's disease (AD), frontotemporal disorders (FTD); or amyotrophic lateral sclerosis (ALS), etc.), and/or primary immunodeficiency.

By “effective amount” is meant the amount of a cell generated as described herein (e.g., a CX3CR1 haploinsufficient HSPC expressing a therapeutic polypeptide generated using a promoterless splice trapping cassette) required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount that enhances engraftment of a transplanted cell in the brain.

“Exogenous nucleic acid molecule” as used herein, refers to a nucleic acid molecule that is not an endogenous nucleic acid molecule, i.e., it is a nucleic acid molecule that does not naturally occur in a cell.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein.

By “hematopoietic stem and progenitor cell (HSPC)” is meant a stem cell or progenitor cell thereof that gives rise to circulating and tissue resident hematopoietic cells in a process known as hematopoiesis.

By “homology arm” is meant a sequence of a vector for homology directed repair (HDR), where the sequence is derived from or homologous to the sequence of an insertion site for the vector. In some embodiments, the length of the homology arm is between at least 10 bp to at least 2000 bp. In some embodiments, the length of the homology arm is between at least 20 bp to at least 1000 bp. In some embodiments, the length of the homology arm is between at least 100 bp to at least 700 bp. In some embodiments, the length of the homology arm is between at least 400 bp to at least 600 bp. In some embodiments, the homology arm is derived from, or homologous to, a nucleotide sequence which is between less than 10 bp away to less than 100 bp away from a double stranded break, such as a double stranded break caused by a Cas nuclease. In one particular embodiment, the homology arm is about 500 bp in length.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “microglia” is meant an immune cell of the central nervous system.

As used herein, “obtaining” as in “obtaining an agent” includes synthesizing, purchasing, or otherwise acquiring the agent.

As used herein, the terms “prevent,” “preventing,” “prevention,” “prophylactic treatment,” and the like refer to reducing the probability of developing a disorder or condition in a subject who does not have, but is at risk of or susceptible to developing, a disorder or condition.

By “reduces” is meant a negative alteration of at least 10%, 25%, 50%, 75%, or 100%.

By “reference” is meant a standard or control condition. In some embodiments, a reference is an unedited HSC or HSPC expressing two copies of CX3CR1.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be, in some embodiments at least about 16 amino acids, at least about 20 amino acids, at least about 25 amino acids, or about 35 amino acids, about 50 amino acids, or about 100 amino acids, or any integer thereabout or therebetween. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or about 250 mM NaCl and 25 mM trisodium citrate. Low stringency hybridization can be obtained in the absence of organic solvent, e.g., formamide, while high stringency hybridization can be obtained in the presence of at least about 35% formamide, and in some embodiments, at least about 50% formamide. Stringent temperature conditions will ordinarily include temperatures of at least about 30° C. at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will comprise less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In some embodiments, wash steps will occur at 25° C. in 30 mM NaCl, 3 mM trisodium citrate, and 0.1% SDS. In other embodiments, wash steps will occur at 42 C in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “subject” is meant a mammal including, but not limited to, a human or a non-human mammal, such as a bovine, equine, canine, ovine, or feline. In some embodiments, the subject has a neurologic or neurometabolic disorder and has undergone myeloablative therapy in preparation for HSC or HSPC transplantation.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transgene” is meant an exogenous nucleic acid molecule, introduced into a host cell, that encodes a polypeptide or polynucleotide to be expressed in the host cell. In some embodiments, the transgene is inserted in a CX3CR1 locus. In some embodiments, the transgene is a therapeutic polypeptide. In some embodiments, polypeptide is one or more of a lysosomal polypeptide associated with lysosomal disorders (e.g., arylsulfatase A (ARSA), galactosylceramidase (GALC), alpha-L-iduronidase (IDUA), iduronate 2-sulfatase (IDS)), polypeptide associated with peroxisomal diseases (e.g., ATP-binding cassette protein subfamily D1 (ABCD1)), microglia-associated polypeptide and/or receptor (e.g., due to colony stimulating factor 1 receptor (CSF1R)), neuromodulating polypeptide (e.g., programmed death-ligand 1 (PD-L1)), polypeptide associated with the pathogenesis of neurodegenerative disorders (e.g., triggering receptor expressed on myeloid cells 2 (Trem2), granulin or progranulin (GRN), superoxide dismutase 1 (SOD1)).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides graphs and an illustration showing that CX3CR1 haplo-insufficient HSPC progeny cells display a unique phenotype in the brain of transplant recipients in standard and competitive settings. A. Engraftment of donor cells in bone marrow (BM) and brain of CD45.1 recipient mice transplanted with either CX3CR1+ or CX3CR1− HSPCs sorted from CX3CR1+/GFPCD45.2 donor mice. B. Representative dot plots of donor derived CD45.2 HSPCs (top) and recipient/support CD45.1 cells (bottom) in the brain of engrafted mice. Brain myeloid subsets (Microglia (p), Transiently Amplifying microglia (TAμ), CNS macrophages (CNS f)) are shown and distinguished based on CD45 and CD11b expression levels. C. Frequency (%) of brain myeloid subsets (μ, Taμ, CNS f) within CD45.2 donor and CD45.1 recipient/support cells in the brain of the transplanted mice are reported. D. Frequency (%) of brain myeloid subsets (μ, Taμ, CNS f) within Cx3cr1−/+ and Cx3cr1+/+ donor derived cells in the brain of transplant recipients sacrificed at 45 days post transplant are shown. Mean values+/−SD and individual values are shown in A, C and D. E. Experimental Scheme. Busulfan conditioned CD45.1 recipients were competitively transplanted with wild type (WT, mCherry+) and Cx3cr1−/+ or Cx3cr1−/− (BFP+) HSPCs either intravenously (IV) or intracerebroventricularly (ICV). Mice were sacrificed at 45 days post transplant. A representative dot-plot showing peripheral blood chimerism of donor derived mCherry+ and BFP+ cells is shown. F, G. Frequency (%) of Cx3cr1+/+ Cherry+ and Cx3cr1−/+(F) or Cx3cr1−/− (G) BFP+ cells within engrafted donor cells in hematopoietic organs (Bone Marrow (BM), Spleen (Spl), and Thymus (Thy)) and brain (BR) of mice competitively transplanted (IV or, when specified ICV), compared to the infused HSPCs (input) is shown. n>5 per group, 3 independent experiments. Mean values+/−SEM are shown. Statistical analysis: Mann Whitney Test; *p<0.05, **p<0.01, ***p<0.001. H. Frequency (%) of brain myeloid subsets (μ, Taμ, CNS f) within Cx3cr1−/+ and Cx3cr1+/+ donor derived cells in the brain of IV or ICV transplant recipients sacrificed at 45 days post transplant. Mean values+/−SD are

FIG. 2 provides micrographs and graphs showing that CX3CR1 haplo-insufficient HSPCs show a qualitative maturation advantage towards microglia-like cells (MLCs) as compared to WT cells. A. Representative reconstruction of a brain slice from a competitively transplanted mouse where the engrafted BFP+Cx3cr1−/+ and the Cherry+Cx3cr1+/+ MLCs are visualized. Nuclei were stained with DAPI. On top, a magnification (40×) showing the morphology of the BFP+Cx3cr1−/+ and the Cherry+Cx3cr1+/+ MLCs in the cortex. B. Scheme displaying macro workflow for branching analysis. Cherry+Cx3cr1+/+ are shown on the top and BFP+Cx3cr1−/+ are shown on the bottom. Confocal images, after maximum intensity projection, were analyzed with a standardized macro through the ImageJ software. Morphological criteria adopted to characterize donor derived microglia cells were applied to each cell, which was thresholded, skeletonized and deprived of cell body. C-E. Branching analysis on the BFP+Cx3cr1−/+ and the Cherry+Cx3cr1+/+ MLCs in the brain of IV or ICV competitively transplanted mice. Cumulative length of branches (C), Complexity Index (CI) (D) and Covered environment Area (CEA)(E) are shown. Mean Values values+/−SEM are shown. F. Example of Sholl Analysis on a MLC analyzed via the ImageJ software. G-H. Correlation between intersection radii and sum intersection parameters obtained from the Sholl analysis on BFP+Cx3cr1−/+ and the Cherry+Cx3cr1+/+ MLCs in the brain of IV (G) and ICV (H) competitively transplanted mice. The vertical and horizontal lines divide the graphs in four quadrants, to describe the cells according to different grade of morphologic complexity, i.e. UR=upper right quadrant, for very complex cells characterized by high sum of intersections and high number of intersecting radii; LL=lower left quadrant, for cells with lower complexity; UL (upper left) and LR (lower right) quadrants for cells displaying intermediate complexity between the LL and the UR quadrants. I. Histograms representing the percentage of cells retrieved in each of the four quadrants displayed in G and H to quantify the data. Histogram bars are layered with UL on top, followed by UR, LR, and LL in that order. Images were acquired via Leica SPE confocal in the cortex region of the brain at 40× magnification. n>50 cells per group, n=3 mice/group. Unpaired t Test with Welch's correction was performed. *p<0.05, **p<0.01, ***p<0.001, ****p<0.0001.

FIG. 3 provides graphs showing that the transcriptional profiling of competitively transplanted HSPCs identifies putative signal transduction pathways underpinning CX3CR1 haploinsufficiency advantage. A. Frequency of donor CD45.2+ cells in the BM and brain of competitively transplanted recipients sacrificed at 15 days post-transplant. B. Percentages of Cx3cr1+/+(white) and Cx3cr1−/+(grey) cells within total donor cells in the BM and brain of competitively transplanted recipients, compared to the infused HSPCs (input). n=8, 3 independent experiments. Mean values+/−SEM are shown C. Two-dimensional UMAP representation of the microglia single-cell transcriptome dataset. Each dot represents a cell, as indicated. D. UMAP plot showing the clusters identified using the Louvain algorithm. E. Bar graph showing the genotype frequency in each cluster, Cx3cr1+/+(left) and Cx3cr1−/+(right). F. Gene ontology analysis on cluster 4, enriched with Cx3cr1−/+ cells, showing a significant upregulation of Cdc42 pathway-related genes, microglia migration and prototypical microglia differentiation pathways.

FIG. 4 provides illustrations and graphs showing that CRISPR/Cas9 and AAV6 mediated targeted integration of a promoter-less cassette allows transgene expression under the control of the endogenous CX3CR1 promoter in human cell lines. A. Schematic representation of the human CX3CR1 locus, with a zoom into intron 4 and exon 5, containing the coding sequence. Target sites of the tested sgRNAs are shown. B. Cutting efficiency measured as percentage of INDELs of different sgRNAs delivered as RNP complexes with CRISPR/Cas9 in RPMI8226 and K562 cells. The TIDE software was used to analyze the spectrum and frequency of indels. C. Schematic representation of the targeted integrations with AAV6 donor templates carrying homology arms matching the chosen sgRNA cutting sites in the CX3CR1 locus. SFFV.YFP—CX exon, promoterless.YFP_SA—CX exon and promoterless.YFP_SA—CX intron donor templates were respectively designed for sgRNA9 (CX exon) and sgRNA5 (CX intron). D. Representative FACS plots of RPMI-8226 and K562 cells nucleofected with CRISPR/Cas9 RNPs with the chosen sgRNAs and transduced with the generated AAV6. GFP expression is indicative of the activity of the promoter (promoterless constructs) or efficiency of targeted integration (SFFV exon and AAVS1 safe harbor control) E. Percentage of GFP assessed by FACS in RPMI-8226 and K562 cells edited with CRISPR/Cas9+AAV6 vectors in the tested and control conditions. F-G. Percentage of targeted alleles assessed by ddPCR on bulk, GFP+ and GFP− sorted RPMI-8226 (F) and K562 (G) edited cells. H. CX3CR1 gene expression evaluated with ddPCR on bulk, GFP+ and GFP− sorted RPMI-8226 edited cells. Data shown as fold on mock. Mean values+/−SD are shown. SFFV denotes spleen focus forming virus (SFFV). AAVS1 denotes the safe harbor control.

FIG. 5 provides an illustration and graphs showing that CRISPR/Cas9 and AAV6 mediated targeted integration of a promoter-less cassette allows transgene expression under the control of the endogenous CX3CR1 promoter in hHSPCs. A. Experimental scheme. After thawing, hHSPCs were kept in pre-stimulation media enriched with a cytokine cocktail for 2 days. Then, cells were nucleofected with sgRNAs delivered as RNP complexes with CRISPR/Cas9 and transduced with the AAV6 donors. Cells were then kept in culture and analyzed for NHEJ, HDR and at FACS to measure the efficiency of editing at the CX3CR1 locus. B. Representative FACS plots of the hHSPCs nucleofected with CRISPR/Cas9 RNP with the chosen sgRNAs and transduced with the AAV6. GFP expression is indicative of the activity of the promoter (promoterless constructs) or efficiency of targeted integration (AAVS1 safe harbor control). C. Percentage of GFP assessed by FACS in hHSPCs edited with CRISPR/Cas9+AAV6 vectors in the tested and control conditions. D. Percentage of targeted alleles assessed by ddPCR in the edited hHSPCs in the tested and control conditions. E. CX3CR1 gene expression evaluated by ddPCR in the edited hHSPCs in the tested conditions, shown as fold on mock. F. CX3CR1 protein expression assessed by FACS in edited hHSPCs in the tested conditions shown as fold on mock. Mean values+/−SD are shown. PGK denotes the 3-phosphoglycerate kinase promoter.

FIG. 6 provides an illustration and graphs showing that CX3CR1 edited hHSPCs repopulate hematopoietic organs and brain of myeloablated immunodeficient recipients showing higher engraftment and transgene expression in the CNS compared to hHSPCs edited at a safe harbor locus. A. Experimental scheme. Busulfan conditioned NSG mice were transplanted IV and ICV with hHSPCs edited at CX3CR1 or at the AAVS1 safe harbor locus. 5 days post-transplant, mice received unmanipulated mouse BM backup for rescue from myeloablation. Mice were bled to analyze peripheral blood chimerism and differentiation of human cells. At 12 weeks post-transplant, mice were sacrificed, and hematopoietic organs and brain were collected for cytometric, molecular and immunofluorescence analysis. Secondary transplants were performed isolating human CD34+ cells from the BM of the primary recipients and transplanting them IV into busulfan conditioned NSG recipients, then sacrificed at 12 weeks post-transplant. B. Frequency of mock and edited hCD45+ cells in peripheral blood at 4, 8, 10 and 12 weeks post-transplant. The lines representing data sets are displayed in the following order from top to bottom at week 12 post-transplant: Mock, CX exon, CX intron, and AAVS1. C. Frequency of mock and edited hCD45+ cells in hematopoietic organs (BM, spleen, thymus) and brain of primary and secondary recipients evaluated at 12 weeks post transplant. D. Percentage of mice with thymus at sacrifice in the different groups of the primary and secondary recipients. E. Percentage of engrafted hCD45+ cells expressing CX3CR1 in hematopoietic organs (BM, spleen, thymus) and brain of mice transplanted with mock or edited hHSPCs. For each set of data points (BM, Spleen, Thymus, or Brain), data points are provided in the following order from left to right: Mock, AAVS1, CX exon, and CX intron. F. Transgene expression and its relative mean fluorescent intensity (MFI) in hCD45+ engrafted in hematopoietic organs (BM, spleen, thymus) and brain of mice transplanted with CX3CR1 edited (CX exon or CX intron) or safe harbor edited (AAVS1) hHSPCs. G. Percentage of targeted alleles (HDR) assessed by ddPCR in tissues isolated from the mice transplanted with CX3CR1 edited (CX exon or CX intron) or safe harbor edited (AAVS1) hHSPCs, compared with targeting efficiency retrieved in the infused cell product (input). Mean values+/−SEM are shown. n>6 per group. Two independent experiments. One-way Anova Multiple comparison with Kruskal-Wallis correction was performed. *p<0.05, **p<0.01, ***p<0.001.

FIG. 7 provides pictures and graphs showing that human HSPCs edited at CX3CR1 locus engrafted in the brain of myeloablated NSG recipients showed a quicker maturation towards a microglia-like phenotype. A. Progeny of CX3CR1 (top) or safe harbor (bottom) edited hHSPCs engrafted in the brain of transplanted recipients and differentiated into microglia-like cells. Signal shows the expression of the transgene in edited cells driven by the endogenous CX3CR1 promoter (top) or PGK promoter (bottom). Engrafted cells identified by hNuclei express Iba-1 marker as endogenous/recipient microglia cells. B-D. Branching analysis performed on brain engrafted microglia-like cells. Cumulative length of branches, Complexity Index (CI) and Covered environment Area (CEA) are shown. Mean Values values+/−SEM are reported. n>80 cells/group. n=3 mice/group. E. Correlation between intersection radii and sum intersection parameters obtained from Sholl analysis performed on CX3CR1 or AAVS1 edited cells engrafted in the brain of transplanted mice. The vertical and horizontal lines divide the graphs in four quadrants, to describe the cells according to different grade of morphologic complexity, i.e. UR=upper right quadrant, for very complex cells characterized by high sum of intersections and high number of intersecting radii; LL=lower left quadrant, for cells with lower complexity; UL (upper left) and LR (lower right) quadrants for cells displaying intermediate complexity between the LL and the UR quadrants. F. Histograms representing the percentage of cells retrieved in each of the four quadrants displayed in figure E to quantify the data. Histogram bars are layered with UL on top, followed by UR, LR, and LL in that order. Images were acquired via Zeiss 980 Confocal acquisition, 20× and 40×, Z-stack. n>80 cells, n=3 mice/group. One-way Anova, Multiple comparison, with Kruskal-Wallis correction was performed. *p<0.05, **p<0.01, ***p<0.001, ****p<0.0001.

FIG. 8 provides graphs showing transplantation of Cx3cr1 haplo-insufficient HSPC in standard and competitive settings. A, B. Engraftment of donor cells in BM (A) and brain (B) of mice transplanted with Cx3cr1−/+ or Cx3cr1+/+ HSPCs at the indicated time points post transplant. Mean values+/−SD are shown. C. Frequency (%) of brain myeloid subsets (μ, Taμ, CNS f) within Cx3cr1−/+ and Cx3cr1+/+ donor derived cells in the brain of transplant recipients sacrificed at 90 and 180 days post transplant are shown. Mean values+/−SD are reported. D,E. Engraftment of donor cells in hematopoietic organs (D) and brain (E) of mice transplanted IV or ICV with Cx3cr1+/+ only, Cx3cr1−/+ only or both (Cx3cr1+/++Cx3cr1−/+) HSPCs sacrificed at 45 days post transplantation. Mean values+/−SD are shown. F. Percentages of Cx3cr1+/+(white) and Cx3cr1−/+(grey) donor derived cells within myeloid and lymphoid (B and T) engrafted cells in the spleen of mice competitively transplanted IV. n>5 per group, 3 independent experiments. Mean values+/−SEM are shown. Statistical analysis: Mann Whitney Test; *p<0.05, **p<0.01, ***p<0.001. G. Engraftment of donor cells in hematopoietic organs and brain of mice transplanted IV with Cx3cr1+/+ only, Cx3cr1−/− only or competitive (Cx3cr1+/++Cx3cr1−/−) HSPCs sacrificed at 45 days post transplantation. Mean values+/−SD are shown.

FIG. 9 provides graphs showing a Branching Analysis on Iba1+ and BFP+Cx3cr1+/− and Cherry+Cx3cr1+/+ engrafted MLCs. A-C. Branching analysis on Cx3cr1−/+ and Cx3cr1+/+ derived MLCs identified by expression of fluorescent markers (barred bar, BFP and Cherry, respectively) or by Iba1 positive staining (grey bar) in the brain of IV or ICV competitively transplanted mice. Cumulative length of branches (A), Complexity Index (CI)(B) and Covered environment Area (CEA)(C) are shown. Mean Values values+/−SD are shown. Values were comparable across the analysis.

FIG. 10 provides an illustration and graphs showing a molecular characterization of edited hHSPCs repopulating myeloablated primary and secondary recipients. A. Graphic scheme strategy of primer design for estimating targeting efficiency by ddPCR at the vector to genome junction region. B. Percentage of CX3CR1 protein expression in hHSPCs at thawing and after 7 days in in vitro culture. Mock and AAVS1 conditions are reported. Mean values+/−SD are shown. C. Frequency of hCD45+ cells in peripheral blood at 4, 8, 10 and 12 weeks post hHSPCs transplant. Mean values+/−SEM are shown. Data is provided in sets of 4 in the following order from left to right: Mock, AAVS1. CX exon, and CX intron. D. Human engrafted cell differentiation towards myeloid (hCD13) and lymphoid (hCD19, hCD3) lineages in bone marrow of mice transplanted with mock and gene edited cells evaluated at 12 weeks post transplantation. hCX3CR1 expression in mock and edited cells is also reported. Mean values+/−SEM are shown. Data within each set of 4 is provided in the following order from left to right: Mock, AAVS1. CX exon, and CX intron. E. Percentage of targeted alleles assessed by ddPCR in spleen (whole tissue, indicated as Spl) and spleen sorted lymphoid (CD19+) and myeloid (CD13+) populations compared with the targeting efficiency of the infused product (input), mean values+/−SEM are shown. F. Percentage of targeted alleles in PBMCs of mice transplanted with mock and gene edited cells evaluated at different time points post transplantation and compared with targeting efficiency of the infused cells. Mean values+/−SEM are shown. G. Percentage of targeted alleles assessed by ddPCR in bone marrow of primary and secondary transplanted mice tissues compared with the targeting efficiency of the infused product (input). Mean values+/−SEM are shown.

### DETAILED DESCRIPTION OF THE INVENTION

As described below, the present disclosure features methods and compositions directed to improved central nervous system (CNS) engraftment and myeloid/microglia differentiation of hematopoietic stem progenitor cells (HPSCs) and enhanced and regulated therapeutic gene expression in their CNS progeny upon transplantation in a subject in need thereof.

This disclosure is based, at least in part, on the discovery that cells that are haploinsufficient for C-X3-C Motif Chemokine Receptor 1 (CX3CR1) generate myeloid progeny and mature more quickly post-transplantation in a subject's brain than do wild-type cells.

This disclosure also provides new and innovative CRISPR/Cas9-based gene editing/gene addition methods at the human CX3CR1 locus that allow the generation of a population of edited HSPCs that are haploinsufficient/knocked-out for CX3CR1 and express a therapeutic transcript under the control of the same locus.

Transplantation of engineered hematopoietic stem/progenitor cells (HSPCs) has shown curative potential for multiple pathologic conditions upon repopulation of hematopoietic compartments, including microglia. To foster the engraftment and differentiation of transplanted HSPCs into microglia-like cells (MLCs), the present disclosure provides a novel gene addition strategy involving CX3CR1, a microglia chemokine receptor that regulates microglial recruitment to sites of neuroinflammation and microglia ontogeny.

The data provided herein shows that transplantation of CX3CR1 haploinsufficient HSPCs resulted in a greater engraftment and differentiation towards MLCs as compared to WT HSPCs in competitive transplantations. The examples herein also provide a potential therapeutic strategy designing a CRISPR-based gene addition at CX3CR1 locus with a promoterless, splice-trapping cassette showing higher engraftment and quicker microglia maturation of CX3CR1-edited human HSPCs transplanted into myeloablated recipients as compared to safe harbor-edited HSPCs, together with a strong transgene expression in the CNS. Without intending to be bound by theory, upregulation of signal-transduction pathways involved in microglia maturation and migration retrieved in CX3CR1 haploinsufficient MLC transcription profile may explain the quantitative and qualitative advantage observed. This strategy could extend applicability of HSPC gene therapy to a broader spectrum of neurometabolic and neurodegenerative diseases.

Transplantation of HSPCs engineered by integrating vectors for the expression of disease-specific therapeutic transcripts has shown unprecedented curative potential in patients affected by monogenic neurometabolic diseases (NMDs) when treated in very early disease stages [1-7]. This benefit is mediated by the myeloid transplanted cell progeny in the CNS that acts not only as vehicle for therapeutics but also as a modulator of neuroinflammation, a mechanism that could be theoretically applied to a broader spectrum of neurodegenerative diseases (NDs) (Milazzo et al. under peer review, Nature Communications). Nonetheless, phenotypic effects in these conditions are delayed after treatment likely due to the slow pace of CNS engraftment and differentiation of the engineered HSPCs into microglia-like progeny cells (MLCs) as compared to the rapid progression of neurodegeneration, that hampers the broad application of this approach. Moreover, unregulated gene expression driven by the currently in-use promoters could not be adequate for achieving therapeutic effects in complex neurodegenerative conditions. In some cases, the requirement for a strong promoter to reach the therapeutic threshold of transgene expression can increase the risk of genotoxicity of the semi-random integrating vectors [8-11]. In other cases, the ectopic expression of the therapeutic gene throughout the whole hematopoiesis could cause long-term unwanted effects or toxicity [12]. Importantly, as neuroinflammation and microglia cell-activation play a crucial role in the pathogenesis and progression of NMDs and NDs [13,14], the use of microglia-specific promoters that could function as sensors of the microenvironment to express therapeutic transcripts by HSPC transplant progeny cells in the CNS could be highly desirable.

Herein is provided a novel targeted gene addition approach at the CX3CR1 locus of HSPCs that may successfully address these limitations and allow extending the application and enhancing the therapeutic potential of HSPC gene therapy for NMDs and NDs. The CX3CR1 gene encodes for the chemokine (C-X3-C) motif receptor 1 (Cx3cr1), highly expressed in microglia. Binding to its unique ligand Cx3cl1, Cx3cr1 regulates several microglia functions, including their recruitment to sites of neuroinflammation, contributing to the regulation of microglia morphology and a subset of inflammatory genes [15]. Several reports propose the Cx3cr1/Cx3cl1 axis as a potentially relevant target for therapeutic interventions in the context of NDs [16 17]. By conducting conventional experiments with a Cx3cr1 haplo-insufficient microglia-reporter model, a unique phenotype of Cx3cr1−/+ HSPCs was discovered that, after transplantation in myeloablated recipients, showed a quantitative and qualitative advantage over wild type counterparts in repopulating the CNS myeloid compartment and maturation towards MLCs. Based on these findings, a targeted gene addition strategy at the CX3CR1 locus of human HSPCs is provided that allows reproducing the unique advantage in CNS repopulation shown by the Cx3cr1−/+ HSPCs and enables a myeloid/microglia specific transgene expression in the progeny of the engineered cells. This strategy is a novel HSPC-based gene therapy platform applicable to several neurodegenerative and neuroinflammatory conditions where a timely, efficient, and properly modulated transgene expression in microglia could be beneficial.

### CX3CR1

CX3CR1, also known as the fractalkine receptor, is a seven-transmembrane domain receptor belonging to the G protein-coupled receptor (GPCR) family. It is expressed in several cell types (e.g., microglia, monocytes, natural killer cells, T cells, and smooth muscle cells). Microglia cells are the only type of cell in the central nervous system that express CX3CR1. CX3CR1 is highly expressed during development and in response to brain damage/pathology.

Being a GPCR, CX3CR1's role is inhibitory as it acts to reduce production of cyclic adenosine monophosphate (cAMP) and prevent the triggering of subsequent signaling cascades mediated by second messengers. The intracellular pathways controlled by CX3CR1 signaling involve mainly phospholipase C (PLC), Phosphoinositide 3-kinase (PI3K), and extracellular-signal-regulated kinase (ERK) regulation, which modulate cell migration, adhesion, proliferation, and survival. Moreover, CX3CR1 is one of the key molecules involved in microglia ontogeny.

Fractalkine (CX3CL1) is the unique ligand for the chemokine receptor CX3CR1 and is expressed either as membrane-bound molecule or in a soluble form. Fractalkine cleavage is mediated by at least two enzymes, ADAM10 and ADAM17, which are active in homeostatic and inflammatory conditions, respectively. Fractalkine acts mainly as adhesion molecule in its membrane-bound form, while it has chemotactic properties towards CX3CR1 in its soluble form. Local production and membrane expression of CX3CL1 and also CX3CR1 are controlled by other cytokines, like TNFα, IL-1, IFNγ, NO, and hypoxia.

Activation of the CX3CR1-CX3CL1 axis leads to maintenance of microglia in a quiescent state and of homeostasis in the neuronal network. Under physiological conditions, CX3CL1 seems to inhibit microglial activation, while in particular conditions a paradoxical promotion of an inflammatory response may occur. Neurons are the greater producers of CX3CL1 in the brain and this axis is important for communication with microglia cells.

Surprisingly, as reported herein below, transplantation of total bone marrow or HSPCs from donor mice haplo-insufficient for the CX3CR1 gene resulted in the generation of microglia like donor cell progeny in the recipients' brain that are more mature (enriched in CD11b^{high} CD45^{low} microglia-like, p cells, versus CD11b^{low} CD45^{high} transiently amplifying, TAμ cells) than the brain cell progeny of standard wild type donor cells. This phenomenon resulted in an unexpected increase in the number of CX3CR1 hemizygous cells within CD11b^{high} CD45^{low} microglia-like μ cells within the transplanted cell progeny. This was paralleled by an increased number of KLS cells within donor BM cells of CX3CR1 haploinsufficient cell transplant recipients, as compared to KLS cell number within donor BM cells of WT cell transplant recipients.

Unexpectedly, in the context of competitive transplantation, CX3CR1 haplo-insufficient donor derived cells contributed to a greater extent as compared to wild type donor cells to the repopulation of the hematopoietic organs and of the brain myeloid compartment of the recipients. In each tested tissue and cell compartment, the frequency of CX3CR1 haplo-insufficient cells was greater than the frequency of WT cells. A branching study performed on the engrafted cells showed that the brain myeloid progeny of CX3CR1^{+/GFP} cells also acquire a more mature microglia-like morphology and express microglia-associated genes at higher levels than the WT cell progeny.

Of note, CX3CR1 hemizygous mice have no obvious phenotype. Thus, there was no reason to expect that the transplant of CX3CR1 hemizygous cells would differ from the transplant of wild type cells.

Thus, the present disclosure provides a CX3CR1 hemizygous or homozygous defective cells for use in transplantation. In some embodiments, the CX3CR1 hemizygous or homozygous defective cell is a hematopoietic stem progenitor cell (HSPC). In some embodiments, the CX3CR1 hemizygous or homozygous defective cell is isolated from a biological sample or is generated via genome editing, targeted gene addition, or using any other method known in the art to knock out a gene. Methods for collecting biological samples and isolating cells (e.g., HSPCs) therefrom are well-known in the art. In some embodiments, the cells are assessed to determine immunocompatability with a subject.

### Expression of Therapeutic Agents

Some aspects of the present invention provide a CX3CR1 hemizygous or homozygous defective cell comprising an exogenous nucleic acid molecule encoding a therapeutic agent (e.g., therapeutic polypeptide or polynucleotide). The therapeutic agent, in some embodiments, is a polynucleotide or a polypeptide. In some embodiments, the polypeptide or polynucleotide may ameliorate a disease (e.g., a neurological or metabolic, or neurometabolic, or neurodegenerative, disease or disorder) or symptom thereof. In some embodiments the nucleic acid molecule encoding the therapeutic agent is integrated into the genome of the CX3CR1 hemizygous or homozygous defective cell. In some embodiments, the nucleic acid molecule encoding the therapeutic agent is inserted into the loci of the missing or disabled CX3CR1 allele. In some embodiments, expression of the exogenous nucleic acid molecule is regulated by the CX3CR1 promoter/enhancer region, consistent with CX3CR1 expression. In some embodiments, the disease treated or ameliorated is any disease which may be treated with HSPC gene therapy. In some embodiments, the disease treated or ameliorated is one or more of lysosomal disorders (e.g., metachromatic leukodystrophy (MLD), Krabbe disease or globoid cell leukodystrophy (GLD), mucopolysarcharidosis type I (MPS I), mucopolysarcharidosis type II (MPS II), mucopolysarcharidosis type IIIA and IIIB (MPS IIIA and B), GM1 gangliosidosis (GM1)), peroxisomal diseases (e.g., X-linked adrenoleukodystrophy (X-ALD), adrenomyeloneuropathy (AMN)), microgliopathies (e.g., due to CSF1R receptor mutations), neuroinflammatory diseases (e.g., multiple sclerosis (MS)), neurodegenerative disorders (e.g., dementias: alzheimer's disease (AD), frontotemporal disorders (FTD); or amyotrophic lateral sclerosis (ALS), etc.), and/or primary immunodeficiency.

The exogenous nucleic acid molecule, in some embodiments, comprises regulatory elements for expressing a transgene. For example, an exogenous nucleic acid molecule may comprise a transgene encoding a therapeutic agent for the treatment of a metabolic and neurological disease and, in some instances, a promoter for expressing the transgene.

In some embodiments, the exogenous nucleic acid molecule may comprise, in addition to a transgene, a detectable label or other marker that allows identification of cells that have been successfully modified or that are derived from cells that have been successfully modified to express the transgene.

### Generation of Hemizygous or Homozygous Defective CX3CR1 Cells

In some embodiments of the present disclosure, an HSPC is edited to remove or otherwise disable one or both functional copies of CX3CR1 to generate HSPCs that are hemizygous or homozygous defective for the gene.

Gene editing is a major focus of biomedical research, embracing the interface between basic and clinical science. “Gene editing” tools can manipulate a cell's DNA sequence at a specific chromosomal locus without introducing mutations at other sites of the genome. This technology effectively enables a researcher to manipulate the genome of a cell in vitro or in vivo.

In one embodiment, gene editing involves targeting an endonuclease to a specific site in a genome to generate a double strand break at the specific location. If a donor DNA molecule (e.g., a plasmid or oligonucleotide) is introduced, interactions between the nucleic acid comprising the double strand break and the introduced DNA can occur, especially if the two nucleic acids share homologous sequences. In this instance, a process termed “gene targeting” can occur, in which the DNA ends of the chromosome invade homologous sequences of the donor DNA by homologous recombination. By using the donor plasmid sequence as a template for homologous recombination, a seamless knock out of the gene of interest can be accomplished. Importantly, if the donor DNA molecule includes a deletion within the target gene (e.g., CX3CR1), homologous recombination-mediated double strand break repair will introduce the donor sequence into the chromosome, resulting in the deletion being introduced within the chromosomal locus. By targeting the nuclease to a genomic site that contains the target gene, the concept is to use double strand break formation to stimulate homologous recombination and to thereby replace the functional target gene with a deleted form of the gene. The advantage of the homologous recombination pathway is that it has the potential to generate seamlessly a knockout of the gene in place of the previous wild-type allele.

Genome editing tools may use double strand breaks to enhance gene manipulation of cells. Such methods can employ zinc finger nucleases, described for example in U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; and 6,479,626; and U.S. Pat. Publ. Nos. 20030232410 and US2009020314, which are incorporated herein by reference); Transcription Activator-Like Effector Nucleases (TALENs; described for example in U.S. Pat. Nos. 8,440,431; 8,440,432; 8,450,471; 8,586,363; and 8,697,853; and U.S. Pat. Publ. Nos. 20110145940; 20120178131; 20120178169; 20120214228; 20130122581; 20140335592; and 20140335618; which are incorporated herein by reference), and the CRISPR (Clustered Regularly Interspaced Short Palindromic Repeats)/Cas9 system (described for example in U.S. Pat. Nos. 8,697,359; 8,771,945; 8,795,965; 8,871,445; 8,889,356; 8,906,616; 8,932,814; 8,945,839; 8,993,233; and 8,999,641; and U.S. Pat. Publ. Nos. 20140170753; 20140227787; 20140179006; 20140189896; 20140273231; 20140242664; 20140273232; 20150184139; 20150203872; 20150031134; 20150079681; 20150232882; and 20150247150, which are incorporated herein by reference). For example, zinc finger nuclease DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. Zinc finger nucleases and TALENs entail use of modular sequence-specific DNA binding proteins to generate specificity for about 18 bases sequences in the genome.

RNA-guided nuclease-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break. The double-strand break is then repaired either by non-homologous end joining, which leads to insertion/deletion (indel) mutations, or by homology-directed repair, which requires an exogenous template and can generate a precise modification at a target locus (Mali et al., Science, Feb. 15, 2013; 339 (6121): 823-6, the contents of which are herein by reference in their entirety). Unlike gene therapy methods that add a functional, or partially functional, copy of a gene to a subject's cells, but retain the original dysfunctional copy of the gene, this system can remove the defect in the dysfunctional copy. Genetic correction using modified nucleases has been demonstrated in tissue culture cells and rodent models of rare diseases.

CRISPR has been used in a wide range of organisms including baker's yeast (S. cerevisiae), zebra fish, nematodes (e.g., C. elegans), plants, mice, and several other organisms. Additionally, CRISPR has been modified to make programmable transcription factors that allow scientists to target and activate or silence specific genes. Libraries of tens of thousands of guide RNAs are now available. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length, with some CRISPR spacer sequences exactly matching sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define eight CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about thirty base pairs in length), which are then inserted into the CRISPR locus near the leader sequence. RNAs from the CRISPR loci are constitutively expressed and are processed by Cas proteins to small RNAs comprising individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes CRISPR transcripts. Interestingly, CRISPR-based phage inactivation in E. coli requires Cascade and Cas3, but neither Cas1 nor Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

As an RNA guided protein, Cas9 requires an RNA molecule to direct the recognition of DNA targets. Though Cas9 preferentially interrogates DNA sequences containing a protospacer adjacent motif (PAM) sequence (i.e., NGG). However, the Cas9-gRNA complex requires a substantial complementarity between the guide RNA (gRNA) and the target nucleic acid sequence to create a double strand break. Synthetic gRNA can be designed to combine the essential RNA sequences for Cas9 targeting into a single RNA expressed with the RNA polymerase type 21 promoter U6 driving expression. Synthetic gRNAs are slightly over 100 bases at the minimum length and contain a portion which is targets the 20 protospacer nucleotides immediately preceding the PAM sequence NGG.

In one approach, one or more cells of a subject are altered to delete or inactivate CX3CR1 using a CRISPR-Cas system. Cas9 can be used to target a CX3CR1gene. Upon target recognition, Cas9 induces double strand breaks in the CX3CR1 target gene. Homology-directed repair (HDR) at the double-strand break site can allow insertion of an inactive or deleted form of the CX3CR1 sequence.

The following US patents and patent publications are incorporated herein by reference: U.S. Pat. No. 8,697,35; 20140170753; 20140179006; 20140179770; 20140186843; 20140186958; 20140189896; 20140227787; 20140242664; 20140248702; 20140256046; 20140273230; 20140273233; 20140273234; 20140295556; 20140295557; 20140310830; 20140356956; 20140356959; 20140357530; 20150020223; 20150031132; 20150031133; 20150031134; 20150044191; 20150044192; 20150045546; 20150050699; 20150056705; 20150071898; 20150071899; 20150071903; 20150079681; 20150159172; 20150165054; 20150166980; and 20150184139. In some embodiments, editing the HSPC to generate hemizygous CX3C3R1 cells comprises inserting an exogenous nucleic acid molecule encoding a therapeutic agent at the CX3CR1 locus. In some embodiments, the exogenous nucleic acid molecule encoding a therapeutic agent inserted at the CX3CR1 locus is a promoterless splice trapping cassette including polynucleotide encoding a splice acceptor site upstream of a sequence encoding a therapeutic agent.

### Expression of Therapeutic Agents in CX3CR1 Hemizygous or Homozygous Defective Cells

Methods are provided herein to modify a CX3CR1 hemizygous or homozygous defective cell to express a therapeutic agent (e.g., neuroprotective polypeptide or polypeptide required to ameliorate a metabolic disorder). In some embodiments, the CX3CR1 hemizygous or homozygous defective cell is modified to incorporate an exogenous nucleic acid molecule encoding a therapeutic agent. The exogenous nucleic acid molecule may be incorporated into the genome of the CX3CR1 cell. The present disclosure also contemplates modifying a CX3CR1 hemizygous or homozygous defective HSPC to incorporate a nucleic acid sequence encoding a therapeutic agent in the edited CX3CR1 allele locus. In this way, the hemizygous or homozygous defective HSPCs are manipulated to express a therapeutic transgene under CX3CR1 locus control.

To express exogenous polynucleotides or polypeptides, nucleic acid molecules encoding the polynucleotides and polypeptides can be inserted into expression vectors by techniques known in the art. For example, double-stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

The present disclosure also includes vectors (e.g., recombinant plasmids) that include nucleic acid molecules (e.g., transgenes) as described herein. The term “recombinant vector” includes a vector (e.g., plasmid, phage, phasmid, virus, cosmid, fosmid, or other purified nucleic acid vector) that has been altered, modified, or engineered such that it contains greater, fewer, or different nucleic acid sequences than those included in the native or natural nucleic acid molecule from which the recombinant vector was derived. For example, a recombinant vector may include a nucleotide sequence encoding a polypeptide, or fragment thereof, operatively linked to regulatory sequences such as promoter sequences, terminator sequences, long terminal repeats, untranslated regions, and the like, as defined herein. Recombinant expression vectors allow for expression of the genes or nucleic acids included in them.

In some embodiments of the present disclosure, one or more DNA molecule having a nucleotide sequence encoding one or more polypeptides or polynucleotides described herein are operatively linked to one or more regulatory sequences, which can integrate the desired DNA molecule into a eukaryotic cell. Cells (e.g., CX3CR1 hemizygous cells) that have been stably transfected or transduced by the introduced DNA can be selected, for example, by introducing one or more markers that allow for selection of host cells containing the expression vector. A selectable marker gene can either be linked directly to a nucleic acid sequence to be expressed or introduced into the same cell by co-transfection or co-transduction. Any additional elements needed for optimal synthesis of polynucleotides or polypeptides described herein would be apparent to one of ordinary skill in the art.

Methods of introducing exogenous nucleic acid molecules into a cell (e.g., CX3CR1 hemizygous cells) are known in the art. For example, eukaryotic cells can take up nucleic acid molecules from the environment via transfection (e.g., calcium phosphate-mediated transfection). Transfection does not employ a virus or viral vector for introducing the exogenous nucleic acid into the recipient cell. Stable transfection of a eukaryotic cell comprises integration into the recipient cell's genome of the transfected nucleic acid, which can then be inherited by the recipient cell's progeny.

Eukaryotic cells (i.e., CX3CR1 hemizygous or homozygous defective HSPCs) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art. While retroviruses systems are generally not compatible with neuronal cell transduction, lentiviruses are a genus of retroviruses well-suited for transducing stem cells as well as neuronal cells. Thus, in some embodiments of the present disclosure, the viral vector system is a lentiviral system. In some embodiments, the viral vector system is an avian virus system, for example, the avian viral vector system described in U.S. Pat. No. 8,642,570, DE102009021592, PCT/EP2010/056757, and EP2430167, the contents of which are incorporated herein by reference in their entirety. In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell.

The components of a viral vector are encoded on plasmids. Because efficiencies of transduction decrease with large plasmid size and to increase the safety of viral vectors (see e.g., Addgene.org/guides/lentivirus/), multiple plasmids that have different viral sequences may be necessary for packaging. For example, in a lentiviral vector system, a first plasmid may comprise a nucleotide sequence encoding a Group antigens (gag) and/or a reverse transcriptase (pol) gene, while a second plasmid encodes regulator of expression of virion proteins (rev) and/or envelope (env) genes. The exogenous nucleic acid molecule comprising a transgene can be packaged into the vector and delivered into a recipient cell where the transgene is integrated into the recipient cell's genome. Additionally, the transgene may be packaged using a split-packaging system as described in U.S. Pat. No. 8,642,570, DE102009021592, PCT/EP2010/056757, and EP2430167.

After the introduction of one or more vector(s), host cells are cultured prior to administration to a subject. Expression of recombinant proteins encoded in the vectors can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography, and electrophoresis. A further purification procedure that may be used for purifying proteins is affinity chromatography using monoclonal antibodies, which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel, by changing pH or ionic strength, for example.

### Hematopoietic Cell Transplantation

HSPCs and/or their progeny that are CX3CR1 hemizygous or homozygous defective can serve as vehicles for therapeutic molecule delivery across the blood brain barrier by contributing to the turnover of myeloid cell populations in the brain. Methods for hematopoietic cell transplantation (HCT) and hematopoietic stem cell (HSC)-based gene therapy are known in the art and have been used to treat patients affected by non-hematological and non-oncological diseases affecting the nervous system, such as peroxisomal disorders and lysosomal storage diseases (LSDs) (Cartier et al. Science 326, 818-823 (2009); Biffi et al. Science 341, 1233158 (2013); Sessa et al. Lancet 388, 476-487 (2016)) and neurological diseases (Simard et al. Neuron 49, 489-502 (2006)). Indeed, HSPCs and/or their progeny can contribute to the turnover of myeloid cell populations in the brain (Ajami et al. Nat Neurosci 10, 1538-1543 (2007); Ajami et al. Nat Neurosci 14, 1142-1149 (2011); Biffi et al. J. Clin. Invest. 116, 3070-3082 (2006); Mildner et al. Nat Neurosci 10, 1544-1553 (2007); Capotondo et al. Proc Natl Acad Sci USA. 109, 15018-15023 (2012). Microglia's role in the progression and outcomes of these disorders has been described (Jeyakumar et al. Brain 126, 974-987 (2003); Wada et al. Proc Natl Acad Sci USA 97, 10954-10959 (2000); Ohmi et al. Proc. Natl. Acad. Sci. USA 100, 1902-1907 (2003); Eichler et al. Ann Neurol 63, 729-742 (2008), the contents of each are incorporated herein in their entirety). In some embodiments, the disease treated or ameliorated is any disease which may be treated with HSPC gene therapy. In some embodiments, the disease treated or ameliorated is one or more of a leukodystrophy (such as, but not limited to, metachromatic leukodystrophy and adrenoleukodystrophy), Mucopolysarcharidosis type I (MPS I) or Hurler syndrome, and/or primary immunodeficiency.

Despite microglia having a developmental origin distinct from that of bone marrow-derived myelomonocytes (Ginhoux et al. Science 330, 841-845 (2010), the contents of which are incorporated herein by reference in their entirety), it has recently been demonstrated that under specific experimental conditions, cells of donor origin showing a microglia-like phenotype and expressing some microglia surface markers could be successfully generated in the brain of mice transplanted with donor HSPCs. Transplant efficiency can be improved with an ablative preconditioning regimen to destroy endogenous microglia progenitors, such as that described in International Application No. PCT/US2017/056774, the contents of which are incorporated herein by reference in their entirety. In some embodiments of the present disclosure, the ablative conditioning regimen comprises administering an alkylating agent to a subject prior to transplantation. In some embodiments, the alkylating agent is busulfan. Busulfan is capable of ablating functionally-defined brain-resident microglia precursors (Capotondo et al. Proc Natl Acad Sci USA. 109, 15018-15023 (2012); Wilkinson et al. Mol Ther 21, 868-876 (2013)).

HSPCs have the capacity to generate new populations of myeloid and microglia cells that can exert therapeutic effects in the central nervous system (CNS). This disclosure provides compositions comprising CX3CR1 hemizygous or homozygous defective HSPCs and enhanced methods for engrafting such cells.

HSPC transplantation generates transcriptionally-dependable microglia through a stepwise process similar to physiological post-natal microglia maturation. CX3CR1 hemizygous or homozygous defective hematopoietic cells able to generate new microglia upon transplantation into myeloablated recipients are retained within human and murine long-term hematopoietic stem cells (HSCs). In some embodiments, microglia-like cells can be generated after intracerebroventricular delivery of CX3CR1 hemizygous or homozygous defective HSPCs, which unexpectedly results in faster and more widespread microglia replacement compared to delivery of wild-type HSPCs. In some embodiments, the CX3CR1 hemizygous or homozygous defective HSPCs of the present disclosure display a more mature microglial phenotype as compared to a reference cell not hemizygous or homozygous defective and/or not containing the promoterless splice trapping cassette of the present disclosure when engrafted in the brain of a subject. In some embodiments, the more mature microglial phenotype may be characterized by one or more of CD45 antigen expression, CD11b antigen expression, length of cell ramifications, complexity of cell ramifications, the total surface area covered by the cell and cell ramifications, and/or number of cell arborizations. In some embodiments, the CX3CR1 hemizygous or homozygous defective HSPCs display upregulation in microglial differentiation and migration pathways when engrated in the brain of a subject, such as, but not limited to, the Cdc42 pathway.

### Pharmaceutical Compositions

Compositions contemplated in the present disclosure include pharmaceutical compositions comprising CX3CR1 haploinsufficient HSPC expressing a therapeutic polypeptide In some embodiments, the therapeutic agent is neuroprotective or is required to replace a missing metabolic enzyme. The pharmaceutical compositions contemplated herein can comprise autogenic or allogenic cells. In some embodiments, the cells are CX3CR1 hemizygous or homozygous defective HSPCs.

The CX3CR1 haploinsufficient HSPC expressing a therapeutic polypeptide as described herein can be administered as therapeutic compositions (e.g., as pharmaceutical compositions). Cellular compositions as described herein can be provided as sterile liquid preparations, e.g., isotonic aqueous solutions, suspensions, emulsions, dispersions, or viscous compositions, which may be buffered to a selected pH. A liquid preparation may be easier to prepare than a gel, another viscous composition, or a solid composition. Additionally, a liquid composition may be more convenient to administer (i.e., by injection). Viscous compositions, on the other hand, can be formulated within the appropriate viscosity range to provide longer contact periods with specific tissues. Liquid or viscous compositions can comprise a carrier, which can be a solvent or dispersing medium comprising, for example, water, saline, phosphate buffered saline, polyol (e.g., glycerol, propylene glycol, liquid polyethylene glycol, and the like), and suitable mixtures thereof.

Sterile injectable solutions can be prepared by incorporating the cells described herein in a sufficient amount of an appropriate diluent. Such compositions may be in admixture with a suitable carrier or excipient such as sterile water, physiological saline, glucose, dextrose, or another carrier or excipient suitable for delivering live cells to a subject. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science,” 17th edition, 1985, incorporated herein by reference, may be consulted to prepare suitable preparations, without undue experimentation.

Additives that enhance the stability and sterility of the cellular compositions, including antimicrobial preservatives, antioxidants, chelating agents, and buffers, can be added. Prevention of the action of microorganisms can be ensured by an antibacterial or antifungal agent including, but not limited to, parabens, chlorobutanol, phenol, and sorbic acid. According to the present disclosure, however, any vehicle, diluent, or additive used must be compatible with the cells.

The compositions can be isotonic, i.e., they have the same osmotic pressure as blood and cerebrospinal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride may be suitable for buffers containing sodium ions.

Viscosity of the compositions, if desired, can be maintained at a selected level using a pharmaceutically acceptable thickening agent. In some embodiments, the thickening agent is methylcellulose, which is readily and economically available and is easy to work with. Other suitable thickening agents include, but are not limited to, xanthan gum, carboxymethyl cellulose, hydroxypropyl cellulose, and carbomer. The concentration of the thickener will depend upon the agent selected and the amount of the agent used. Suitable carriers and other additives may be chosen depending on the route of administration and the nature of the dosage form (e.g., a liquid dosage form can be formulated into a solution, a suspension, a gel, or another liquid form, such as a time release formulation or liquid-filled form).

In some embodiments, the compositions may include the CX3CR1 hemizygous or homozygous defective HSPCs disclosed herein and a suitable culture medium. In some embodiments, the culture medium is a medium suitable for cryopreservation. In some embodiments, the HSPCs are cryopreserved. Cryogenic preservation is useful, for example, to store the HSPCs for future use, e.g., for therapeutic use, or for other uses, e.g., research use. The HSPCs may be amplified and a portion of the amplified HSPCs may be used and another portion may be cryogenically preserved. HSPCs produced using the methods as disclosed herein can be cryopreserved according to routine procedures. For example, cryopreservation can be carried out on from about one to ten million cells in cryopreservation medium which can include a suitable proliferation medium, 10% BSA and 7.5% dimethylsulfoxide. HSPCs are centrifuged. Growth medium is aspirated and replaced with cryopreservation medium. HSPCs are resuspended as spheres. Cells are slowly frozen, by, e.g., placing in a container at −80° C. Frozen HSPCs are thawed by swirling in a 37° C. bath, and resuspended in saline or cryopreservation medium.

An effective amount of cells to be administered can vary for the subject being treated. In one embodiment, between about 104 to about 10⁸ cells, and in another embodiment between about 105 to about 10⁷ cells are administered to a subject. In some embodiments, a human is administered a dose of at least about 0.1×10⁶ cells/kg, at least about 0.5×10⁶ cells/kg, at least about 1×10⁶ cells/kg, at least about 5×10⁶ cells/kg, at least about 1×10⁷ cells/kg, at least about 5×10⁷ cells/kg.

The skilled artisan can readily determine the amounts of CX3CR1 hemizygous or homozygous defective cells and optional additives, vehicles, and/or carrier in compositions to be administered. In one embodiment any additive (in addition to the cell(s)) is present in an amount of about 0.001% to about 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD₅₀ in a suitable animal model, e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

### Methods of Treatment

The present disclosure provides methods of treatment for a subject in need thereof by administering a CX3CR1 haploinsufficient HSPC expressing a therapeutic polypeptide, or a pharmaceutical composition comprising the cell, to the subject. In some embodiments, the subject in need of treatment has or is suspected of having a metabolic or neurological disease.

In embodiments, a therapeutic polypeptide of the disclosure useful in the treatment of a disease of the central nervous system.

A health care professional may diagnose a subject as having a metabolic or neurological disease by the assessment of one or more symptoms of disease in the subject. The present disclosure provides methods of treating a metabolic or neurological disease or symptoms thereof that comprise administering to a subject (e.g., a mammal, such as a human) a therapeutically effective amount of a cell hemizygous for CX3CR1 that may or may not express a therapeutic polypeptide. In some embodiments, the cell is an HSPC. In some embodiments, the cell is a microglial progenitor cell. Thus, the method in some embodiments comprises administering to the subject a therapeutically effective amount of a cell described herein sufficient to treat a metabolic or neurological disease or symptom thereof, under such conditions that the disease is treated.

The methods herein include administering to the subject (including a subject identified as in need of such treatment) an effective amount of cells described herein, or a composition comprising such cells as described herein to produce such effect. Such treatment will be suitably administered to a subject, particularly a human, suffering from, having, susceptible to, or at risk for, a metabolic or neurological disease, or a symptom thereof. In some embodiments, the methods herein include administering to the subject (including a subject identified as in need of such treatment) an effective amount of a compound described herein, or a composition described herein to produce such effect.

In some embodiments, the cell or the composition comprising the cell is administered to a subject in a targeted manner. For example, in some embodiments, a composition comprising a CX3CR1 haploinsufficient HSPC expressing a therapeutic polypeptide is administered directly to a subject's brain. In some embodiments, the composition is delivered directly to the brain via intracerebroventricular (ICV) administration. In some embodiments, the composition is delivered in this manner to the lateral ventricles of the subject's brain. In some embodiments, the composition is administered to the subject through an intrathecal (IT) route, such as, but not limited to, the intrathecal lumbar (ITL) route.

Alternatively, the composition may be delivered systemically, such as by intravenous administration. Cells administered in such a manner must traverse the blood brain barrier prior to engrafting in the subject's brain. Other modes of administration (parenteral, mucosal, implant, intraperitoneal, intradermal, transdermal, intramuscular, intracerebroventricular injection, intravenous including infusion and/or bolus injection, and subcutaneous) are generally known in the art. In some embodiments, cells are administered in a medium suitable for injection, such as phosphate buffered saline, into a subject. Because the cells being administered to the subject are intended to repopulate microglial cells, intracerebroventricular administration may be advantageous as other routes of administration require crossing the blood brain barrier.

In some embodiments, the cell hemizygous or homozygous defective for CX3CR1 is modified to express a therapeutic agent. In some embodiments, the genome of the cell hemizygous or homozygous defective for CX3CR1 is modified to have a nucleic acid encoding a therapeutic agent at the CX3CR1 locus, such that the cell comprises one functional copy of the CX3CR1 gene and one functional copy of the nucleic acid molecule encoding the therapeutic agent. In some embodiments, the therapeutic agent is a neuroprotective agent. In such embodiments, engraftment of transplanted CX3CR1 hemizygous or homozygous defective cells that express a therapeutic agent in a subject's brain provides a population of cells that express a therapeutic agent. But because the transplanted cells are meant to replace endogenous cells (i.e., microglial cells), in certain embodiments, methods of treating a subject having, susceptible to, or at risk of developing a metabolic or neurological disease further comprise administering to a subject prior to administering a CX3CR1 hemizygous or homozygous defective HSPC expressing a therapeutic agent, an agent for ablating endogenous cells, such as microglia. In some embodiments, the agent is an alkylating agent. In some embodiments, the alkylating agent is busulfan. In some embodiments, nanoparticle delivery of alkylating agents may be effective in creating a suitable environment for engraftment of transplanted HSPCs, as described in International Application No. PCT/US2017/056774, the contents of which are incorporated herein by reference in their entirety.

### Kits

The present disclosure contemplates kits for the treatment or prevention of a metabolic or neurological disease and for delivery of a transgene to a cell. In some embodiments, the kit comprises a composition comprising a cell hemizygous or homozygous defective for CX3CR1. In some embodiments, the cell hemizygous or homozygous defective for CX3CR1 is modified to express a therapeutic agent. In some embodiments, the genome of a cell hemizygous defective for CX3CR1 is modified to have a nucleic acid encoding a therapeutic agent at the CX3CR1 locus, such that the cell comprises one functional copy of the CX3CR1 gene and one functional copy of the nucleic acid molecule encoding the therapeutic agent. In some embodiments, the genome of a cell homozygous defective for CX3CR1 is modified to have a nucleic acid encoding a therapeutic agent at the CX3CR1 locus, such that the cell comprises no functional copies of the CX3CR1 gene and at least one functional copy of the nucleic acid molecule encoding the therapeutic agent. The kit can include instructions for a treatment protocol, reagents, equipment (test tubes, reaction vessels, needles, syringes, etc.), and standards for calibrating or conducting the treatment protocol. The instructions provided in a kit according to the present disclosure may be directed to suitable operational parameters in the form of a detectable label or a separate insert. Optionally, the kit may further comprise a standard or control information so that the test sample can be compared with the control information standard to determine if a consistent result is achieved. In some embodiments, the kit includes a nanoparticle for ablative conditioning of endogenous microglial cells.

In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, an agent of the invention is provided together with instructions for administering the agent to a subject having or at risk of developing a metabolic or neurological disease or disorder of the central nervous system. The instructions will generally include information about the use of the composition for the treatment or prevention of the disease or disorder. In other embodiments, the instructions include at least one of the following: description of the therapeutic agent; dosage schedule and administration for treatment or prevention of a neurological disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Cx3cr1 Haplo-Insufficient HSPC Progeny Cells Display a Unique Phenotype in the Brain of Transplant Recipients

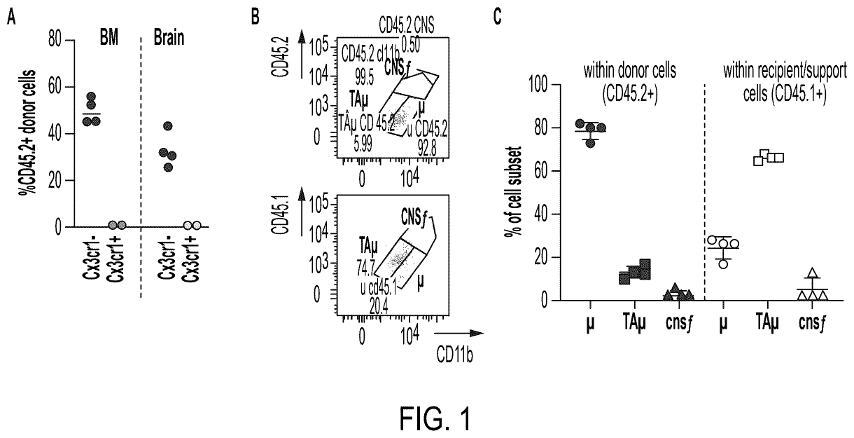

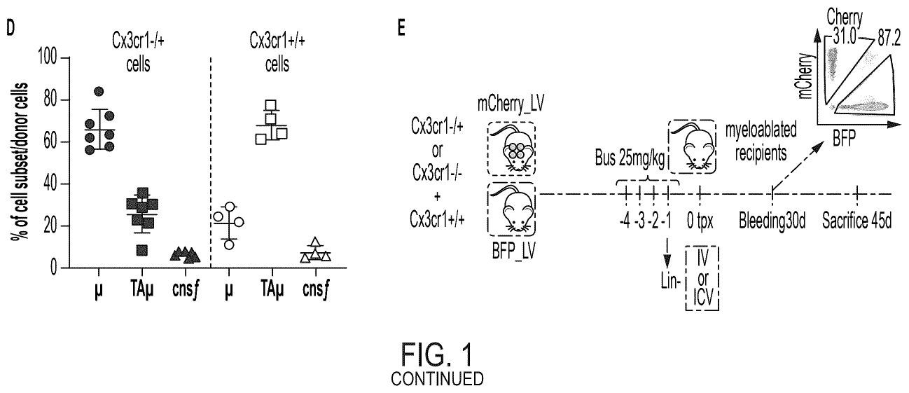

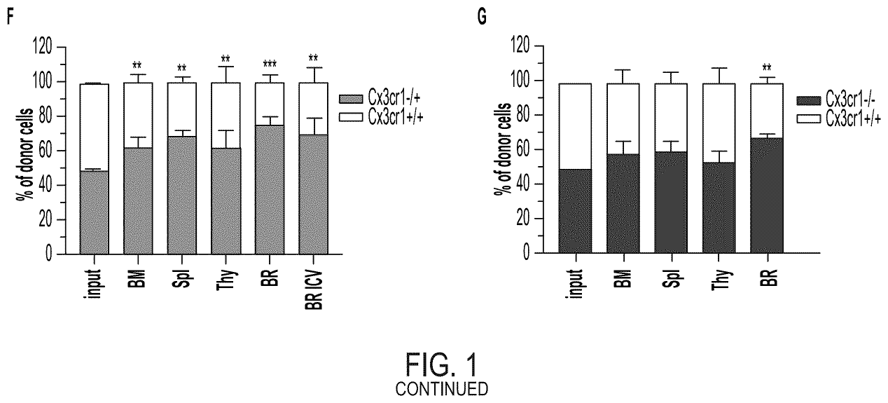

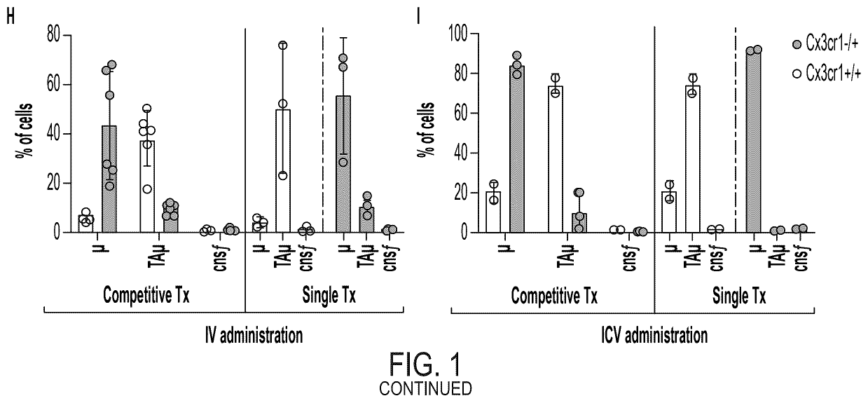

Tracking experiments were performed employing cells retrieved from CD45.2 donor mice that are haplo-insufficient for Cx3cr1 because of GFP insertion at the locus (Cx3cr1−/+), with consequent GFP signal in cells expressing Cx3cr1, such as microglia [18]. GFP+ and GFP− cells were FACS sorted from the lineage negative (Lin−) HSPC pool of Cx3cr1−/+ donors and independently transplanted them in busulfan conditioned CD45.1 recipients to evaluate their capability of engraftment in the bone marrow (BM) and brain. At sacrifice, 45 days later, only mice receiving GFP− Lin− cells showed a robust donor cell engraftment in the BM and in the brain (FIG. 1A); no donor-derived cells were retrieved in mice receiving the GFP+ Lin− fraction, indicating that cells expressing Cx3cr1 are not capable of engraftment in transplant recipients.

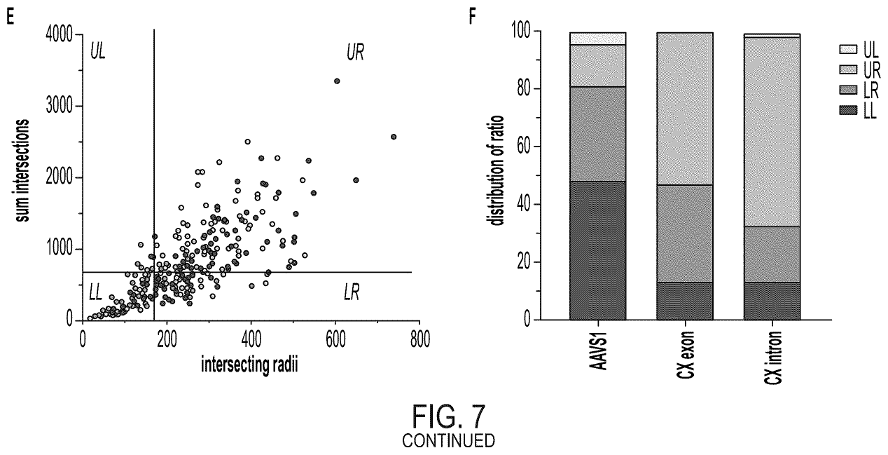

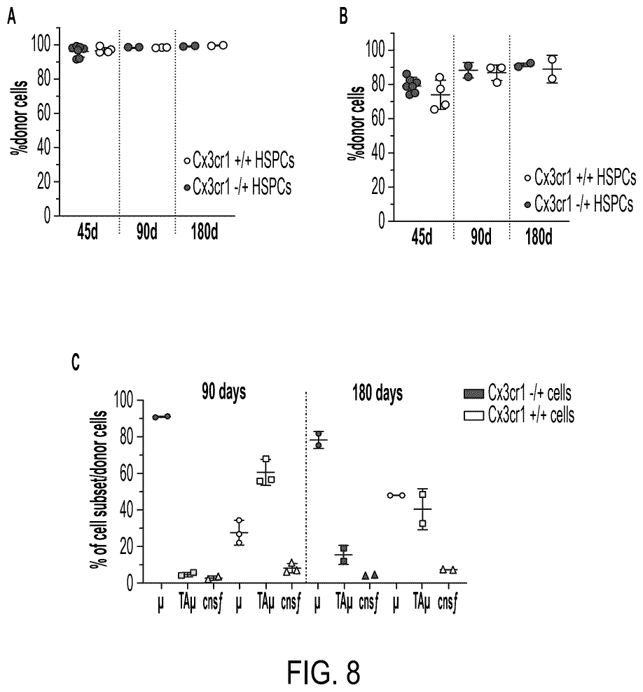

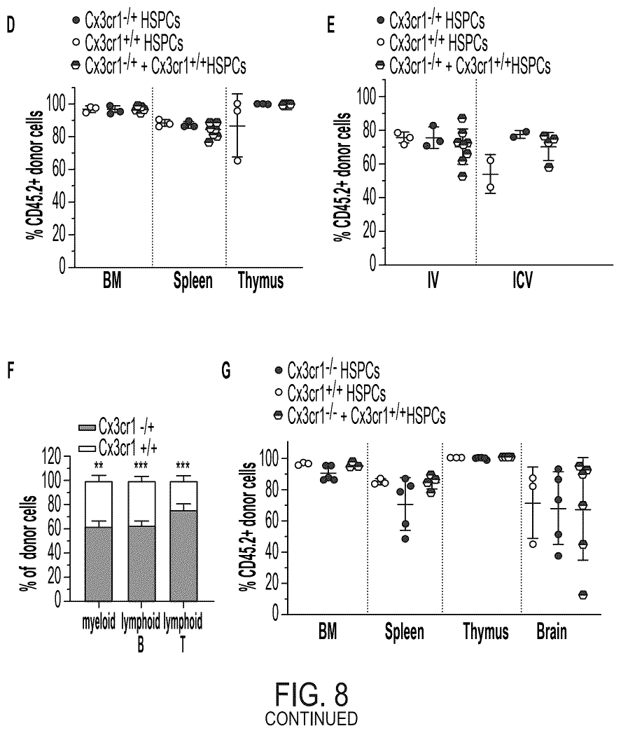

Unexpectedly, flow cytometry analysis revealed that the majority of cells derived from Cx3cr1−/+ donors in the brain of transplant recipients showed the characteristics, as far as CD45 and CD11b antigen expression are concerned (FIG. 1B), of mature microglia (p) cells from naïve control mice (FIG. 1C), rather than of more immature transiently amplifying μ (TAμ) cells that usually prevail in the transplant setting, particularly in early stages [19]. This was particularly evident when comparing within individual animals the cells derived from the CD45.2 Cx3cr1−/+ donors with the CD45.1 counterpart derived from the CD45.1 BM support and/or from endogenous microglia [19](FIG. 1C). It was hypothesized that the haploinsufficiency of Cx3cr1 could have contributed to the observed more mature phenotype of the transplant-derived MLCs in the recipients. To test this hypothesis, additional dedicated experiments were performed. Firstly, HSPCs isolated from Cx3cr1−/+ donor mice were transplanted into CD45.1 myeloablated recipients that were then analyzed at different time points post-transplant. CD45.1 myeloablated recipient mice transplanted with HSPCs from Cx3cr1+/+ wild-type (WT) donors were used as controls. At all the time points of analysis, WT and Cx3cr1−/+ cells engrafted equally in the BM and brain of the recipient mice (FIG. 8(A),(B)). Consistently with the previous observation, also in this setting, μ-like cells prevailed over TAμ cells in the brain of mice transplanted with Cx3cr1−/+, but not with WT HSPCs, particularly at the earliest post-transplant time points (FIG. 1(D), 8(C)).

### Example 2: Cx3cr1 Haplo-Insufficient HSPCs Outcompete WT Cells in Repopulating Hematopoietic Organs and Brain in a Competitive Transplantation Setting

To further investigate the modality of Cx3cr1−/+ HSPC repopulation of the recipient brain myeloid compartment and differentiation into MLCs upon transplant, a competitive transplantation experiment was performed. CD45.2+Cx3cr1−/+ and WT HSPCs, labelled by lentiviral vector transduction with different fluorescent markers, were co-transplanted at a 1:1 ratio intravenously (IV) or intracerebroventricularly (ICV) in CD45.1 busulfan conditioned recipients (FIG. 1(E)). As control, mice were transplanted with either WT or Cx3cr1−/+ HSPCs alone. Input HSPCs were comparable for phenotype (94,53±2,5% Lin− cells in Cx3cr1−/+ HSPCs; 93,9±3,3% Lin− cells in WT HSPCs), marker-gene expression (99.05±0.78% BFP+ in Cx3cr1−/+ HSPCs; 98,75±0.78% mCherry+ in WT HSPCs) and clonogenic potential (CFUs counts: 50±7,26 CFUs for Cx3cr1−/+ HSPCs; 50,25±5,18 CFUs for WT HSPCs) across separate experiments. At sacrifice, a high and comparable engraftment of the donor cells was observed in hematopoietic organs (IV administration) and brain (IV and ICV administration) of transplant recipients in all experimental groups (FIG. 8(D),(E)); no donor cells were observed in the hematopoietic organs of mice transplanted ICV (data not shown), in line with previous data [20,21]. Unexpectedly, the proportion of Cx3cr1−/+ versus WT cells within the cells of donor origin was uneven at sacrifice as compared to input cells, which were transplanted at a 1:1 ratio. Indeed, the Cx3cr1−/+ cells prevailed over the WT counterpart in hematopoietic organs (60% vs 40%) and, even more strikingly, in the brain of the recipients, where the cells derived from the Cx3cr1−/+ HSPCs constituted up to the 80% of the total donor cells (FIG. 1(F)). The proportion of Cx3cr1−/+ versus WT cells within hematopoietic cell lineages throughout tissues reflected the one detected on total donor cells, except than in CD3+T lymphocytes, for which a greater unbalance between the BFP+Cx3cr1−/+ cells and the mCherry+WT cells was observed (almost 80% vs 20%) (FIG. 8(F)). Similar results were obtained by co-transplanting Cx3cr1−/− HSPCs, completely defective in Cx3cr1, with WT cells, even if in the absence of a dose effect (FIG. 1(G), 8(G)). Interestingly, and consistently with the initial data, also in this transplantation setting, the progeny of the Cx3cr1−/+ HSPCs at flow cytometry mostly showed a μ-like phenotype rather than TAμ cell features, at odds with the progeny of the Cx3cr1+/+ HSPCs that showed a pattern of CD45 and CD11b antigen expression consistent with standard transplants, with prevalence of TAμ cells over p (FIG. 1(H)). The initial data of the single transplantation setting were also here confirmed in the controls, with the majority of the Cx3cr1−/+ cell progeny showing CD45 and CD11b expression consistent to p cells (and of the Cx3cr1+/+ cell progeny showing features of TAμ)(FIG. 1(H)).

### Example 3: The MLC Progeny of Cx3cr1 Haplo-Insufficient HSPCs Shows a More Mature Morphology than their WT Counterpart in a Competitive Transplantation Setting

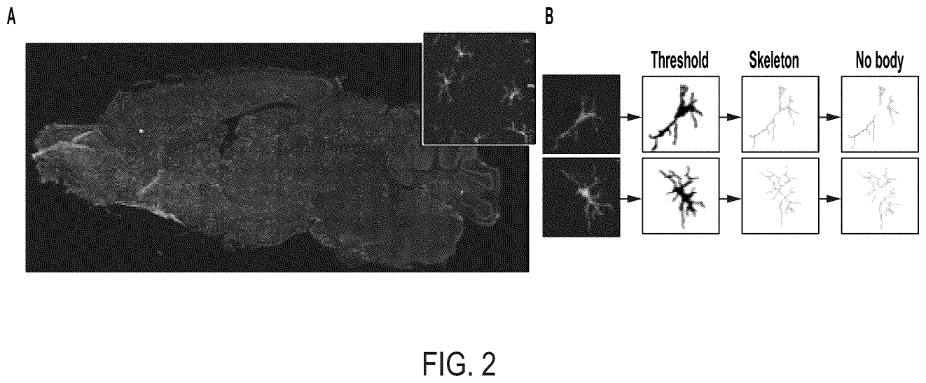

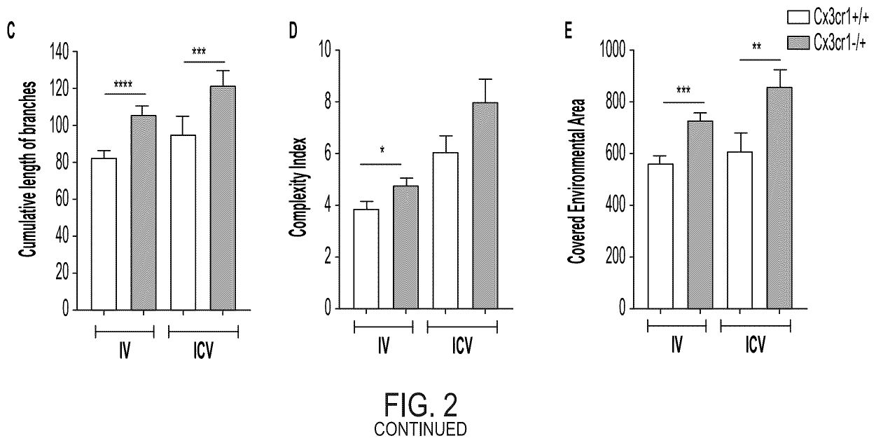

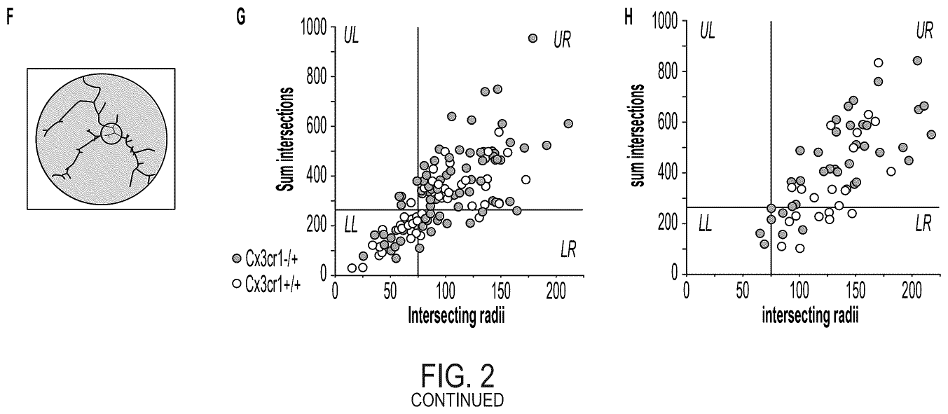

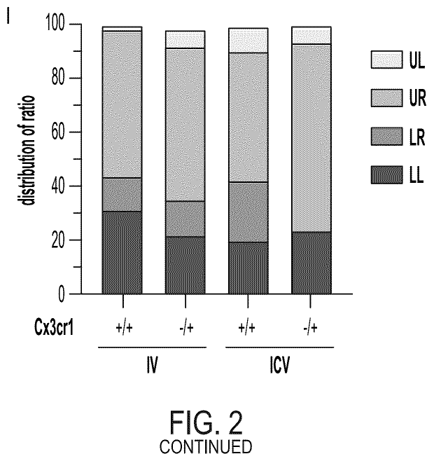

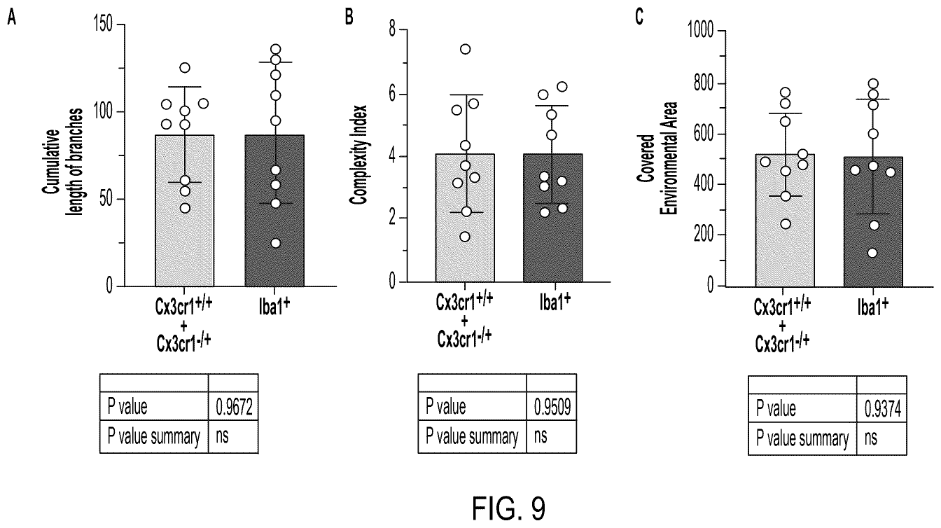

Based on these early findings, a branching study was performed to characterize the WT and Cx3cr1 haplo-insufficient cell progeny in the brain of competitive transplant recipients. The analysis was run on brain slices, identifying transplant-derived cells by fluorescent marker expression (FIG. 2(A),(B)), once confirmed its ensured overlapping results with the analysis of Iba-1 staining (FIG. 9). Cell morphology was described using parameters previously employed in the literature for describing microglia morphology [22]: i) the total length of all cell ramifications (Sum Length); ii) the Complexity Index (CI), defined as the ratio between the total number of branches of each cell (where a branch is the length of process between two nodes) and the number of its primary ramifications; iii) the Covered Environment Area (CEA), namely the 2D total surface covered by the cell ramifications and defined as the area of the polygon formed by linking the extremities of its processes (FIG. 2(B)). The branching analysis revealed interesting differences between the transplanted cell progenies. Cx3cr1−/+ HSPC progeny showed a significantly higher total length of ramifications as compared to the WT HSCP progeny in both the IV and ICV competitive transplant settings (FIG. 2(C)). The analysis of CI and CEA also revealed a greater complexity of ramifications and a larger size of the Cx3cr1−/+ MLCs versus WT MLCs (FIG. 2(D),(E)). These results were more pronounced in the IV transplantation setting as compared to the ICV one, suggesting a greater competition in the former setting. This is in line with previous data indicating that the ICV route favors per se the differentiation of HSPCs towards MLCs [21]. An automated Sholl analysis [22] was also performed to study the radial distribution of microglia branches around the cell body (FIG. 2(F)). The sum intersections and number of intersection radii were selected as parameters to quantitatively characterize donor derived cells for their spatial distribution and complexity of the cell arborization pattern based on the morphology and on the extension of cell arborizations, respectively. The analysis showed that cells derived from Cx3cr1−/+ HSPCs covered a larger surface and displayed more arborizations as compared to the WT cell progeny both in the IV and in the ICV transplantation settings (FIG. 2(G),(H),(I)). Indeed, by plotting these two parameters (sum intersections and number of intersection radii) in a bidimensional correlation, it was observed that the progeny of Cx3cr1−/+ transplanted HSPCs was more abundantly distributed than the Cx3cr1+/+ cell progeny in the upper right—UR—quadrant that corresponds to the cells covering the greater surface and displaying more arborizations (FIG. 2(G),(H),(I)).

### Example 4: Single Cell Transcriptome Profiling Reveals a Microglia Signature in Cx3Cr1 Haploinsufficient Cells

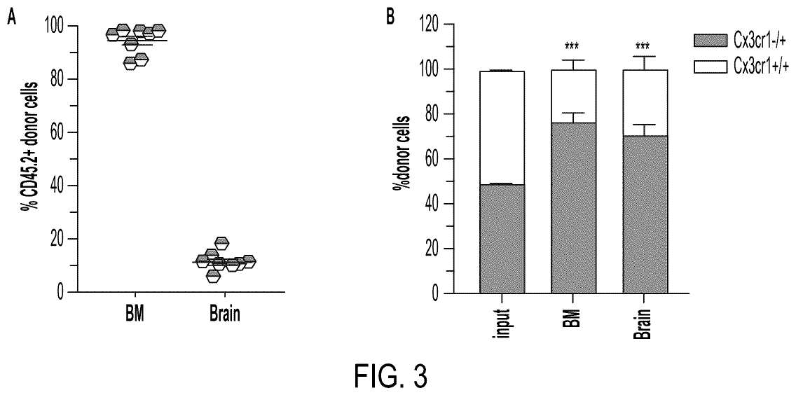

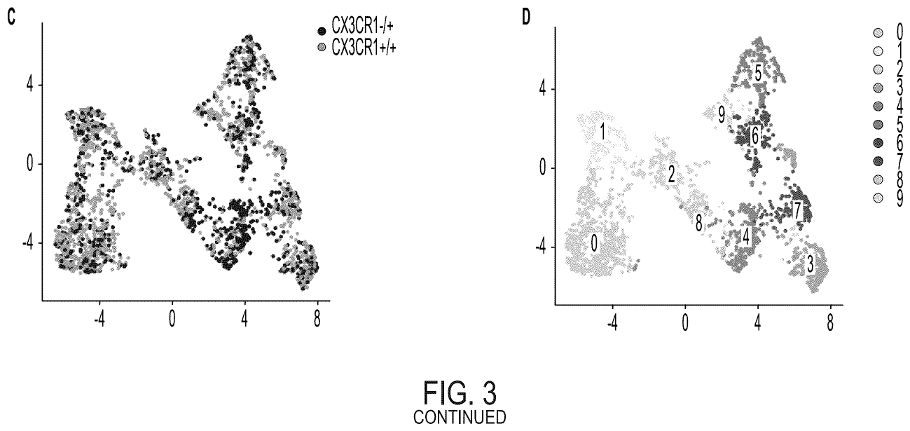

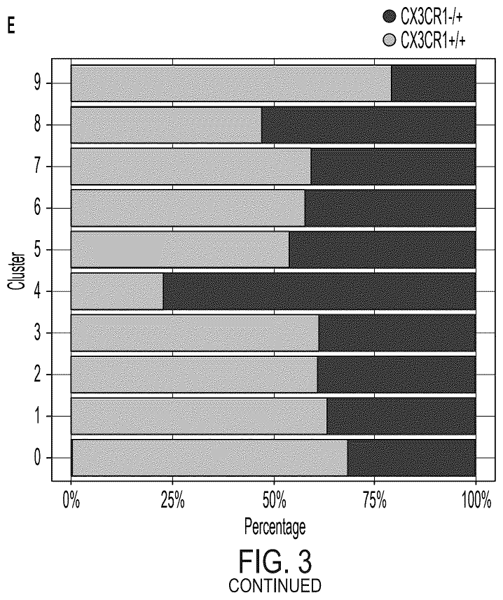

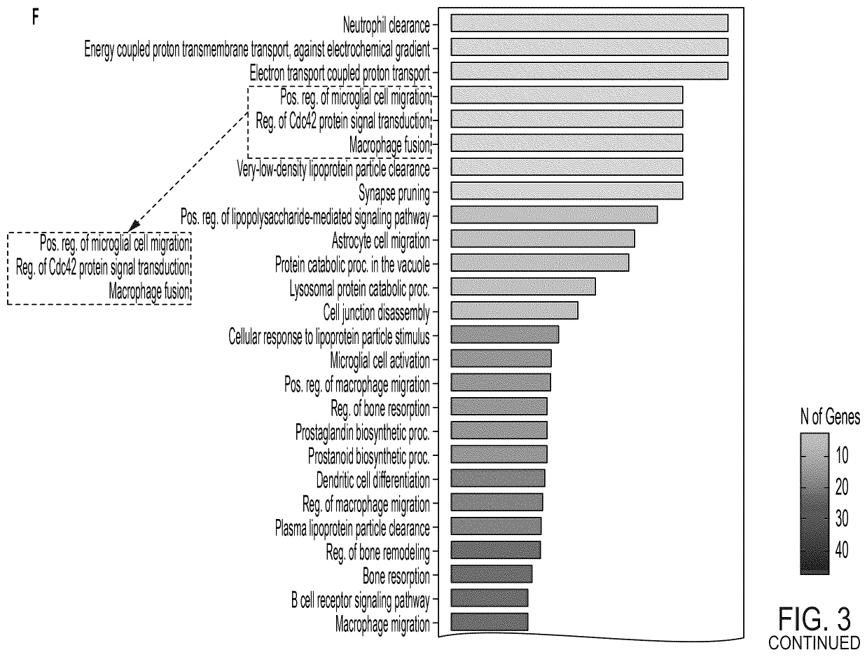

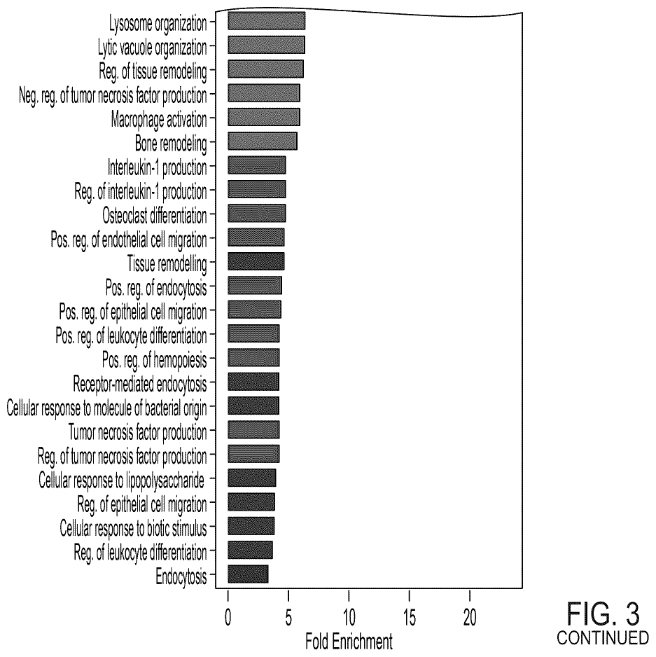

To obtain some insight on the mechanism that could be responsible for the potential of Cx3cr1 haplo-insufficient HSPCs in populating the brain of myeloablated recipients with donor-derived mature MLCs at a faster pace than WT cells, a transcriptomic analysis was performed at single cell resolution on FACS-sorted MLCs isolated from competitively transplanted mice, using the same experimental scheme described above in FIG. 1(E). At sacrifice, after extensive perfusion, BM and brain samples were collected for flow cytometry and MLCs from the brain were also FACS-sorted, separating WT vs haploinsufficient cells according to fluorescent marker expression to perform a transcriptomic analysis at single cell resolution with the 10×Genomics platform. Transplanted mice were euthanized at an early time point after transplant (15 days) to study the early dynamics of the cell reconstitution process in the brain. At this time point of analysis, flow cytometry showed that engraftment in the BM had already reached a plateau, while in the brain the donor cell frequency was still low (FIG. 3(A)). Consistently to previous data, the analysis confirmed an higher frequency of Cx3cr1 haplo-insufficient versus WT cells within the engrafted donor population, both in the BM and brain, also at this early time point of evaluation (FIG. 3(B)). The distribution of the Cx3cr1 haplo-insufficient and WT single-cell transcriptome profile was evaluated in a two-dimensional UMAP plot (FIG. 3(C)), on which several clusters were identified (FIG. 3(D)). Interestingly, a substantial enrichment in Cx3cr1 haplo-insufficient MLCs characterized cluster 4 over the others (FIG. 3(E)). GSEA was next performed using gene scores derived from the cluster 4 marker analysis and identified a significant upregulation of signal transduction pathways associated to prototypical microglia differentiation and migration pathways (FIG. 3(F)). In particular, upregulation of genes related to Cdc42, a small GTPase of the Rho-subfamily, which regulates signaling pathways controlling diverse cellular functions including cell morphology, migration, endocytosis and cell cycle progression (FIG. 3(F)) was observed. This observation may suggest that Cx3cr1-deficiency could be associated to a greater activation of the Cdc42 pathway during the process of myeloid cell differentiation and generation of MLCs following HSPC transplant that could favor their maturation towards a microglia-like phenotype and their distribution throughout the brain.

### Example 5: Optimization of CRISPR/Cas9-Based Gene Editing and Targeted Gene Addition at the Human CX3CR1 Locus

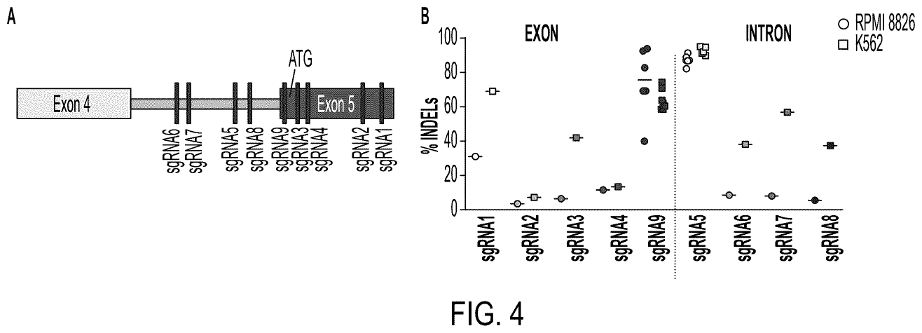

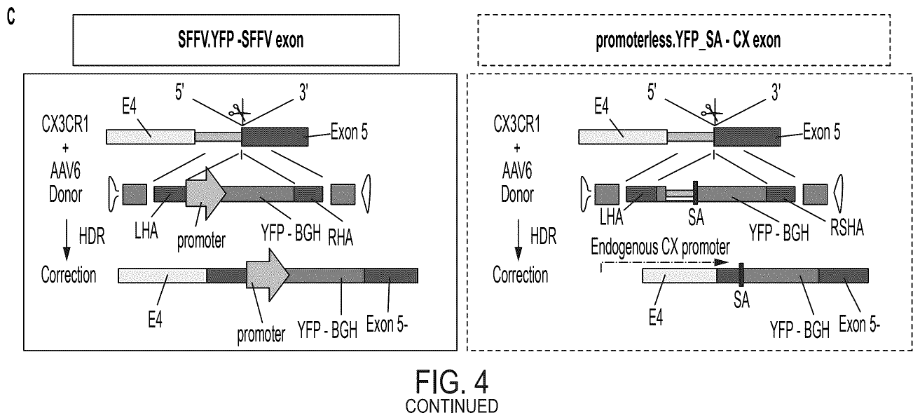

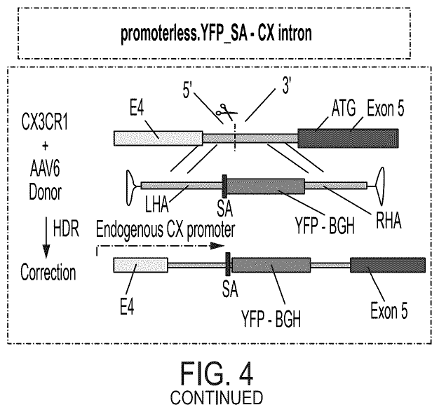

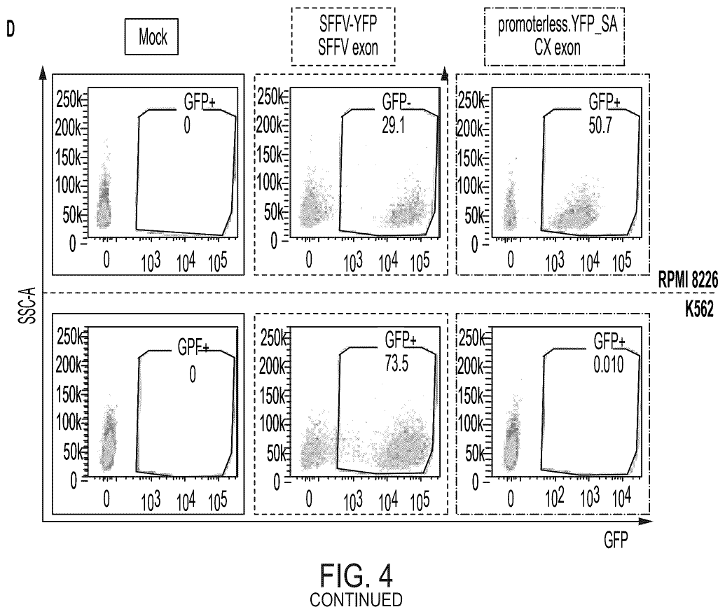

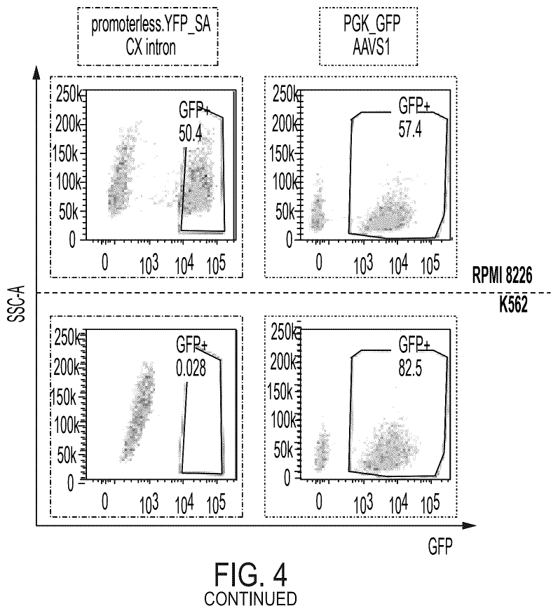

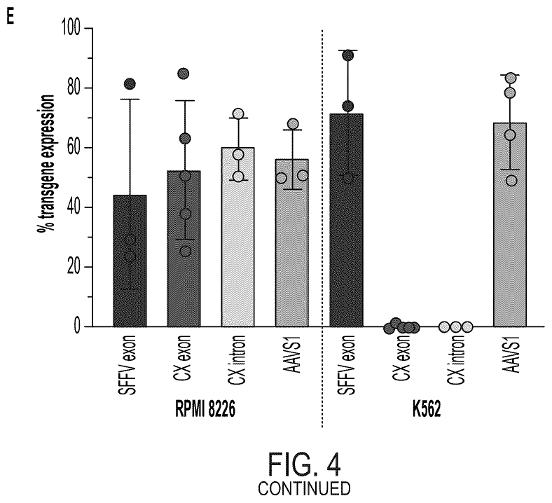

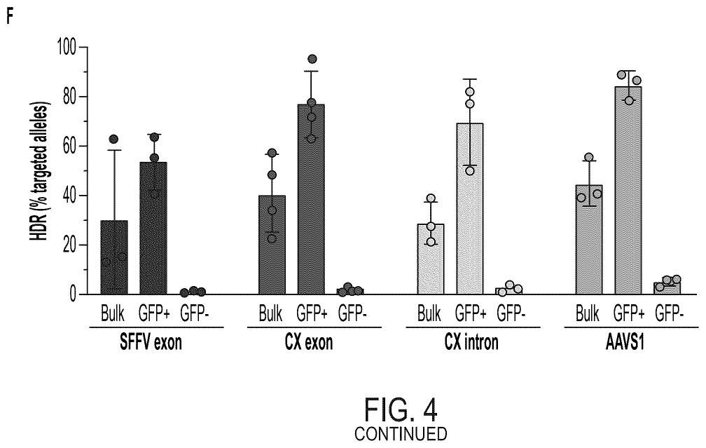

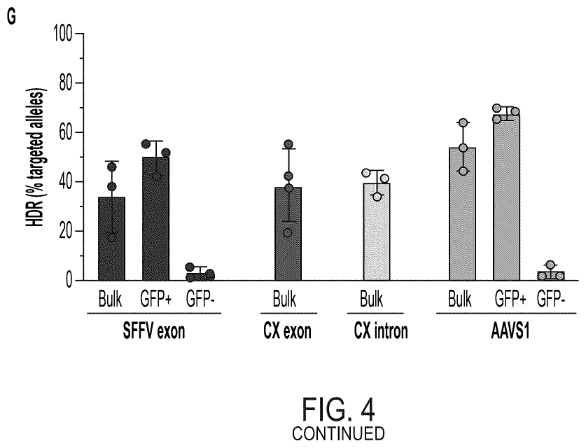

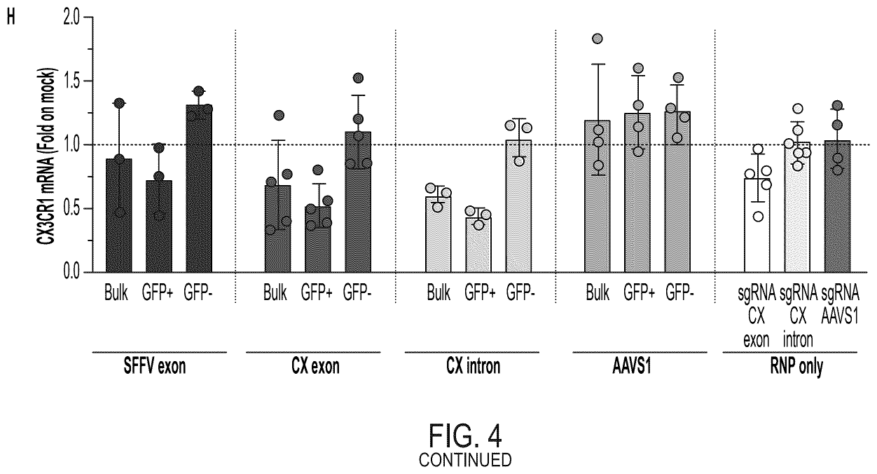

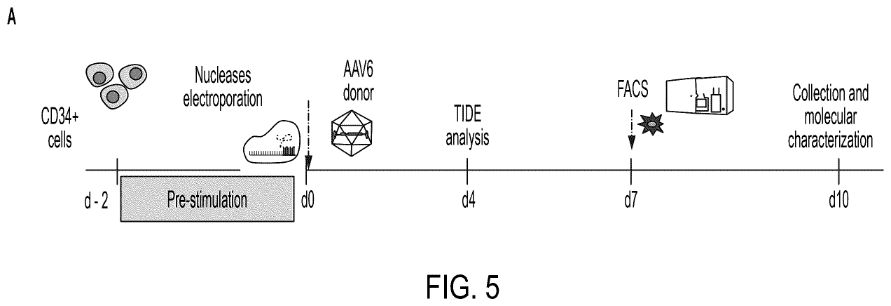

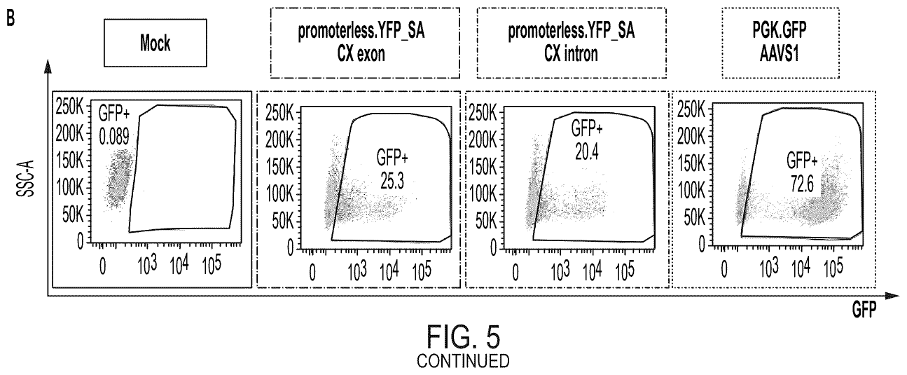

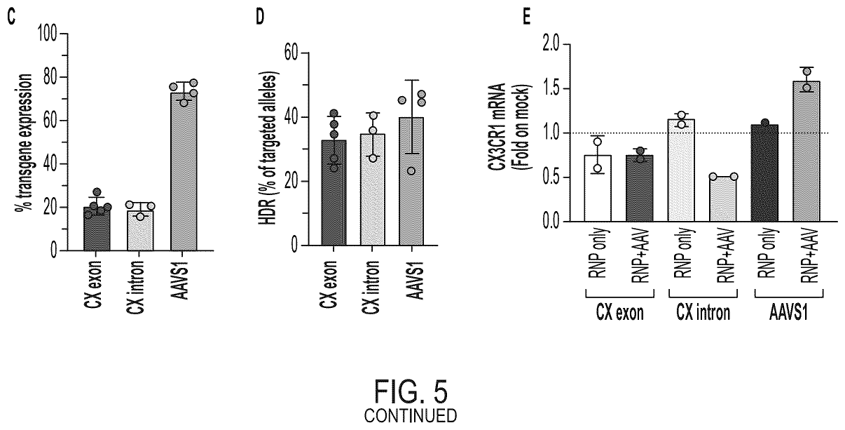

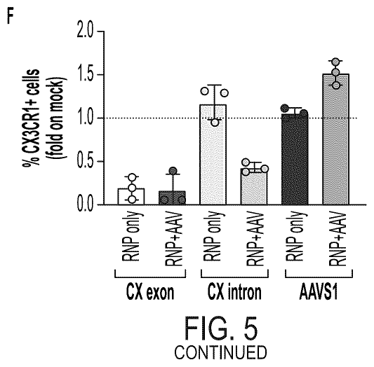

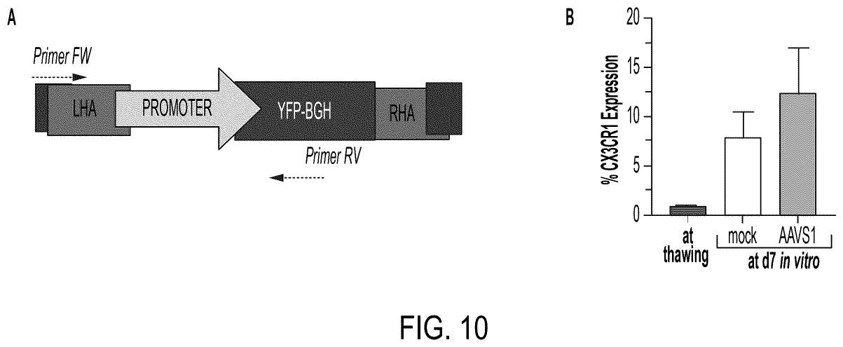

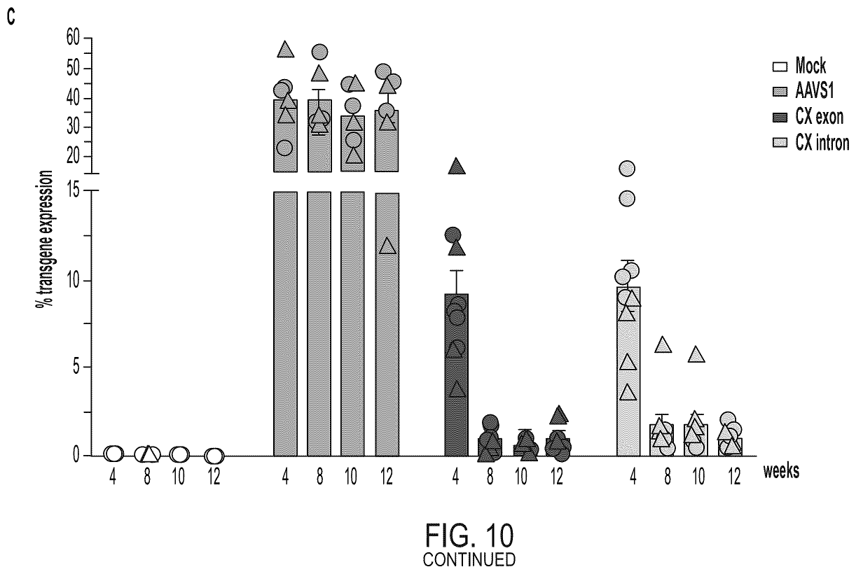

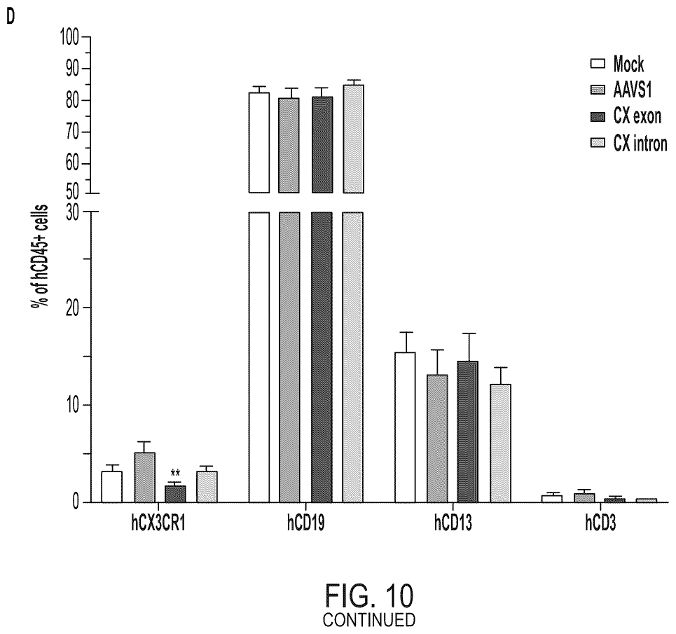

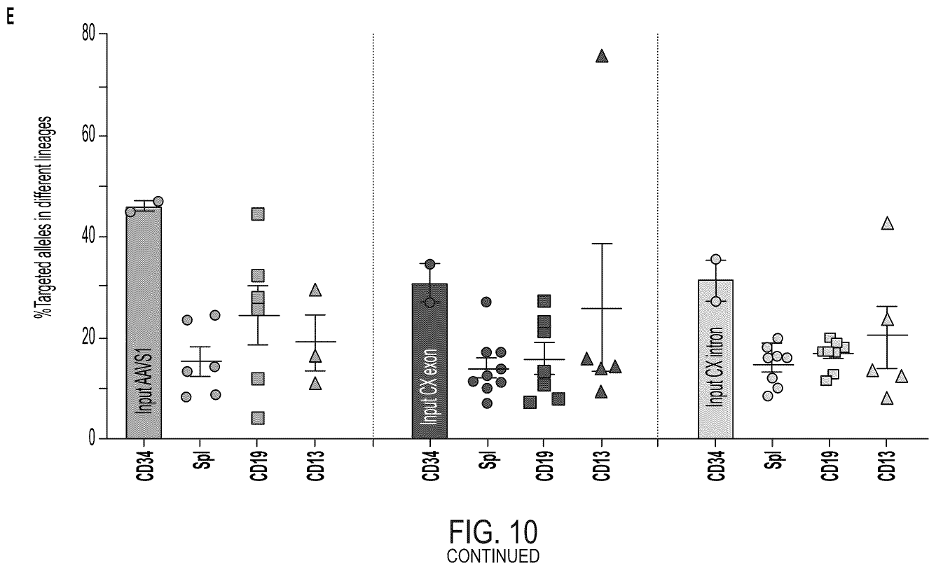

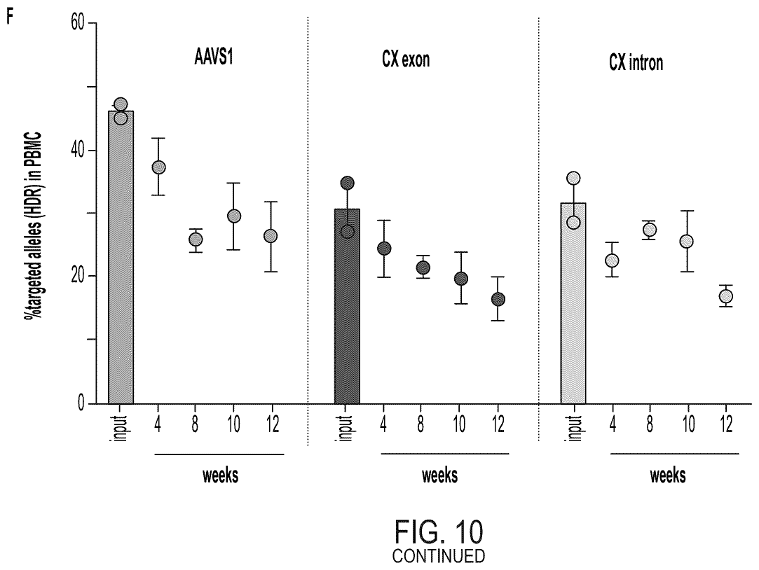

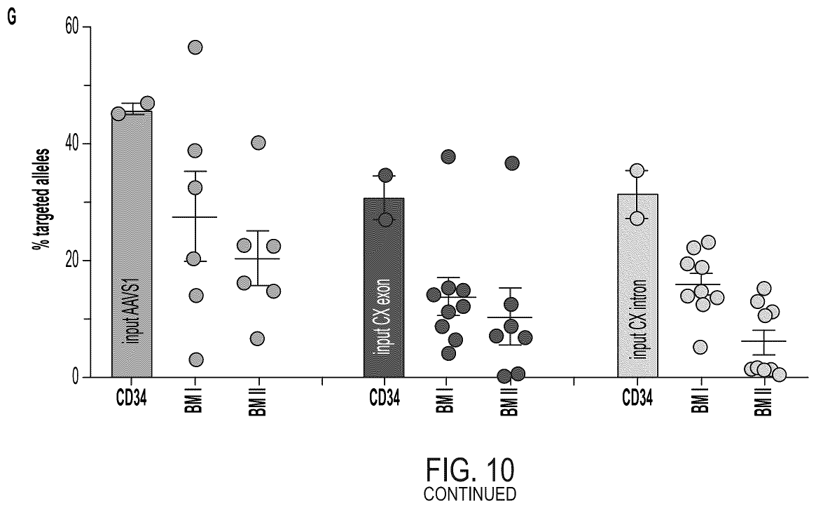

A CRISPR-Cas9 based gene editing strategy was developed to insert an exogenous transgene expression cassette into the human CX3CR1 locus while concomitantly knocking out the expression of the edited allele. Indeed, this approach could allow: i) exploiting the positive effects of CX3CR1 haplo-insufficiency in human HSPCs to enhance their ability to repopulate the brain myeloid compartment with a progeny that could efficiently differentiate towards MLCs, and ii) expressing a therapeutic transgene under the control of the endogenous CX3CR1 promoter, which is highly active in steady-state microglia and even more robustly expressed upon their activation in pathological conditions. To edit the human CX3CR1 locus, a targeting strategy specific for the intron 4 and one specific for the exon 5 were designed and compared, which contains the entire gene coding sequence [23,24]. The exonic approach would result in gene disruption with both NHEJ-mediated indels and homology directed repair (HDR)-mediated targeted integration, thus likely leading to full CX3CR1 knock out in most of the treated cells. On the contrary, the intronic approach would disrupt CX3CR1 expression mainly after HDR-integration of the transgene cassette, while most of the NHEJ-mediated indels should only minimally affect endogenous gene expression. Thus, this latter approach would mostly mimic an haploinsufficiency phenotype. For both approaches, a panel of different single guide RNAs (sgRNAs) was designed and tested (FIG. 4(A)) and one sgRNA was identified for each approach that resulted in the highest cutting efficiency (sgRNA5 for the intronic region and sgRNA9 for the exon; FIG. 4(B)). For the both the approaches, a promoter-less donor DNA template was designed that contained a splice-trapping cassette encoding for the Yellow Fluorescent Protein (YFP) flanked by homology arms (HA) that match the nuclease cutting sites. For the exon, a similar donor template was designed where the expression of the reporter was driven by the constitutive spleen focus forming virus (SFFV) promoter (SFFV exon) (FIG. 4(C)). Donor templates were cloned and produced as Adeno-Associated Virus type 6 (AAV6) vectors to obtain efficient delivery in primary HSPCs. As control, previously validated sgRNA/AAV6 reagents [25] were used that mediate the insertion of a constitutive PGK.GFP cassette into the AAV Site 1 (AAVS1), a paradigmatic safe harbor for targeted transgene insertion [26]. To validate the editing strategies, the RPMI 8226 and the K562 cell lines were edited, which express or not the CX3CR1 gene, respectively. In K562 control cells, as expected, transgene expression was observed only in the SFFV exon and AAVS1 conditions, where the expression of the fluorescent reporter is driven by the constitutive promoters encoded by the integrated cassette (FIG. 4(D),(E)). On the contrary, edited RPMI 8226 cells showed expression of the fluorescent reporter in all the conditions, reaching up to 80% of transgene positive cells (FIG. 4(D),(E)). Digital droplet PCR (ddPCR) with primers designed at the vector-to-genome junction for the different constructs tested (FIG. 10(A)) confirmed a comparable and high targeted integration efficiency (average of 40% of the targeted alleles) for all the tested pairs in both cell lines, molecularly verified also in cells sorted according to the expression of the fluorescent reporter (FIG. 4(F),(G)). These data confirm that the promoter-less cassette allowed specific transgene expression by the CX3CR1 promoter, which is active in RPMI 8226 cells and not in the K562 cell line. Importantly, targeted integration at both the exonic and intronic sites resulted in transcriptional downregulation of CX3CR1 in RPMI 8226 cells (FIG. 4(H)). An established editing protocol based on CRISPR-Cas9/gRNA electroporation and AAV6 transduction was then used to edit and obtain targeted addition of the cassettes in human CD34+ HSPCs (hHSPCs) [25](FIG. 5(A)). A dim, but consistent expression of the fluorescent reporters was observed by flow cytometry in the conditions targeted with the promoter-less cassettes at the CX3CR1 locus, likely reflecting the activity of the CX3CR1 promoter in this heterogenous cell population when cultured in vitro (FIG. 5(B),(C), 10(B)). Reporter gene expression in the AAVS1 condition was consistent with previous data [25](FIG. 5(B),(C)). Analysis of targeting efficiency in the edited cells by ddPCR showed an efficient integration (up to 45% of targeted alleles) of all the tested cassettes (FIG. 5(D)), resulting in transcriptional and protein downregulation in HSPCs edited at the CX3CR1 locus (FIG. 5(E),(F)). Importantly, in the CX3CR1 intron condition, targeted insertion allowed regulated transgene expression and CX3CR1 knocking-out only in HDR edited cells, while NHEJ-indels did not significantly impact CX3CR1 expression, thus reproducing a haploinsufficiency condition.

### Example 6: Engraftment and Differentiation of hHSPCs Edited at the CX3CR1 Locus Along Serial Transplants

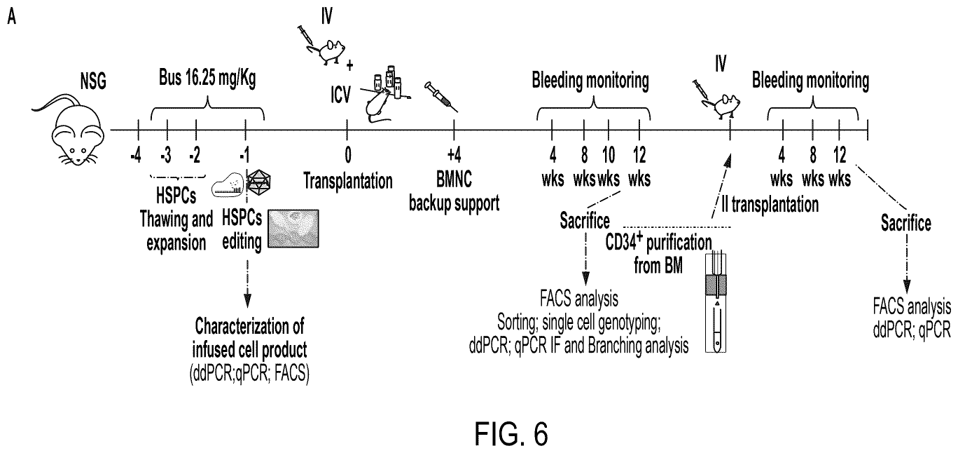

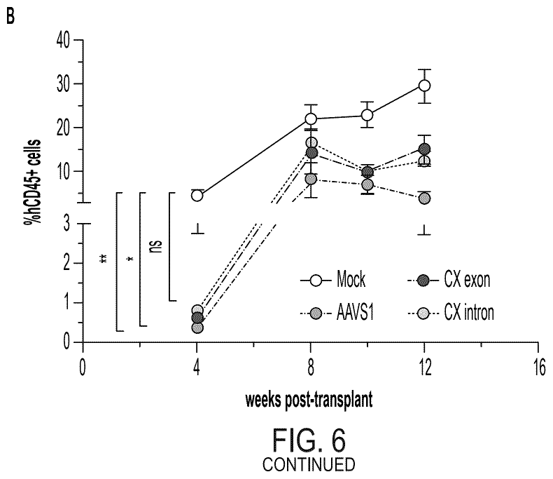

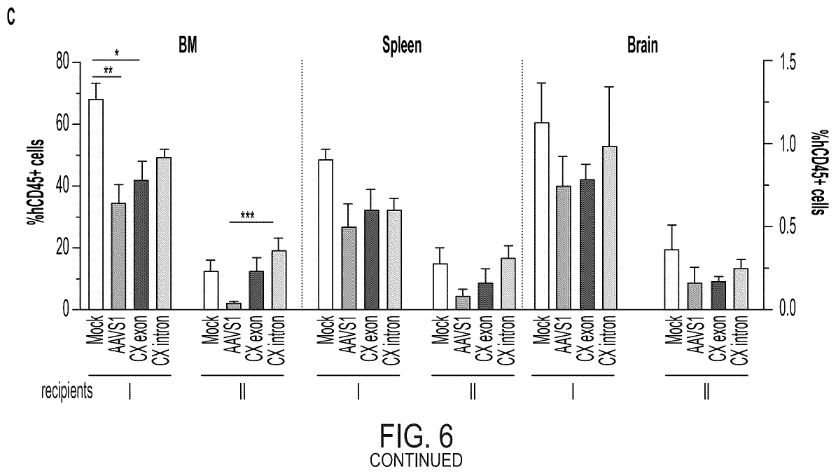

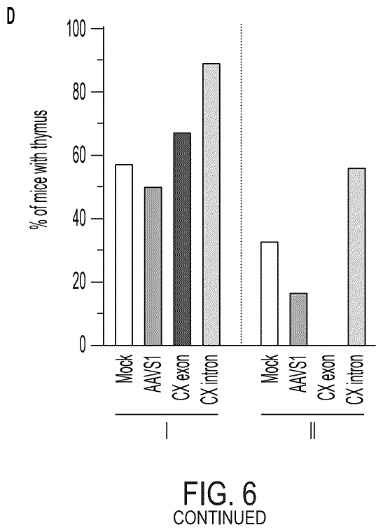

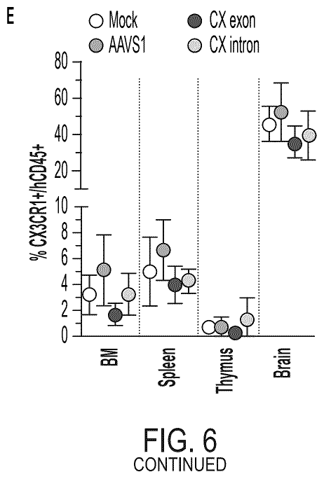

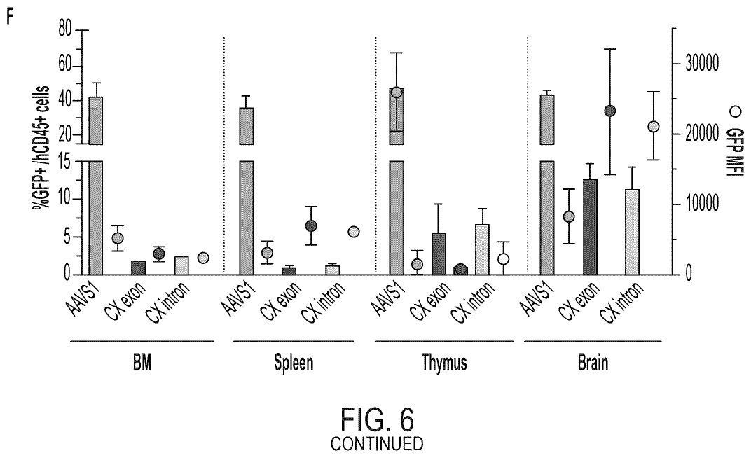

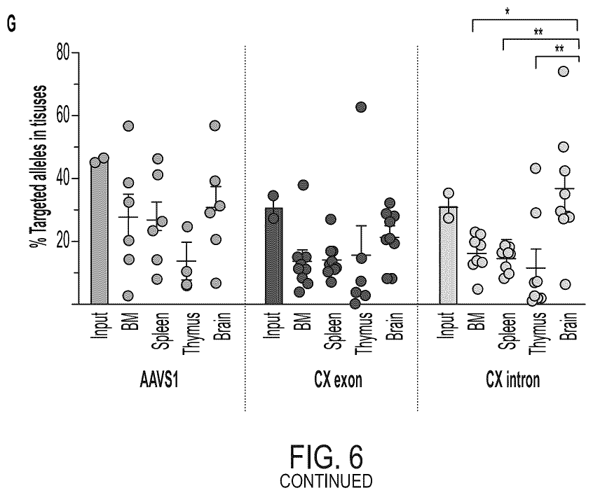

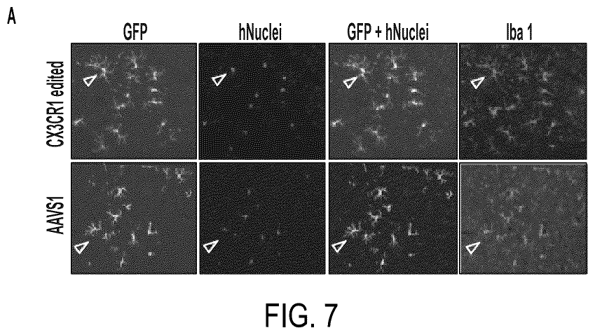

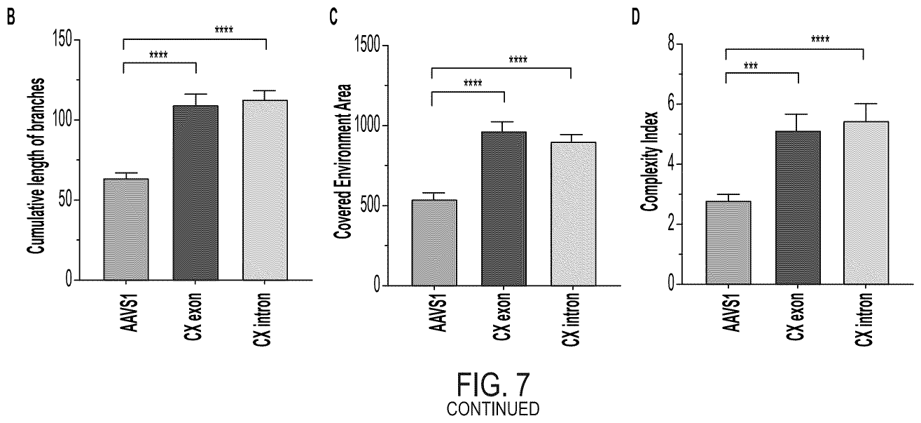

The functional features of the CX3CR1 edited hHSPCs were tested in vivo by transplantation into busulfan myeloablated nonobese diabetic (NOD)-severe combined immunodeficiency (SCID)-gamma (NSG) immunodeficient recipients. A pool of CD34+ hHSPCs was edited as described above in two independent experiments. Edited cells were then administered to the NSG recipients IV and ICV, the latter to enhance their engraftment in the CNS [21]. Unedited (mock) and AAVS1 edited hHSPCs were used as controls (FIG. 6(A)). The presence of human cells and their differentiation in the recipients were assessed by longitudinal blood cytofluorimetric and molecular analyses. At every time point of analysis, a lower engraftment of the edited hHSPCs as compared to the mock-treated cells was observed, as expected [25](FIG. 6(B)). Interestingly, however, mice transplanted with hHSPCs edited at the CX3CR1 locus showed a higher engraftment as compared to AAVS1 edited hHSPCs, particularly at the earliest time point post-transplant (4 weeks), suggesting that CX3CR1 editing in hHSPCs could be associated to a greater engraftment ability (FIG. 6(B)). On average 40% of the engrafted human cells in the AAVS1 edited group expressed the transgene, and this fraction remained stable over time in the peripheral blood, indicating efficient and successful editing on repopulating hHSPCs (FIG. 10(C)). Notably, mice transplanted with the CX3CR1 edited cells showed overall low levels of fluorescent reporter expression in the human cells circulating in peripheral blood, with the highest expression at the earliest timepoint of observation, when the majority of the graft consists of myeloid cells [27]. This data may suggest that the integrated promoter-less cassette, also in vivo, could be expressed with a specific pattern that possibly reflect the hematopoietic reconstitution dynamics (FIG. 10(C)). At steady state hematopoiesis, mice were euthanized to collect hematopoietic organs and brain after extensive perfusion. CD34+ human progenitor cells were isolated from their BM to perform secondary transplantations (by IV cell delivery) into busulfan conditioned NSG recipients (FIG. 6(A)). At sacrifice of all the primary and secondary recipients, the progeny of the transplanted human cells was detected in hematopoietic organs (BM, spleen, thymus) and brain (FIG. 6(C)). Overall, and consistently with peripheral blood data and the literature, the edited hHSPCs showed a lower engraftment than the mock-treated cells. Interestingly, also in tissues, the hHSPCs edited at the CX3CR1 locus showed a higher engraftment than the hHSPCs edited at the AAVS1 locus and, particularly for the hHSPCs edited in the intron, more similar to the mock treated cells, especially in the BM (FIG. 6(C)). A tendency towards a better engraftment of the CX3CR1 edited cells was observed also in the brain, where engraftment was relatively low, as expected in this xenogeneic chimeric setting [21](FIG. 6(C)). Interestingly, in most of the mice transplanted with CX3CR1 intron-edited hHSPCs the thymus was present and repopulated by the human cells, at odds with the other groups (FIG. 6(D)). Engrafted human cells were also characterized for their immunophenotype, showing a multilineage differentiation in all the experimental groups (FIG. 10(D)). Low percentages of CX3CR1+ human cells were retrieved in hematopoietic organs, while more than 50% of the brain-associated human cells expressed CX3CR1 (FIG. 6(E)). Interestingly, a consistently lower % of CX3CR1+ cells was detected in the CX3CR1-edited hHSPC recipients, possibly reflecting the modulation of protein expression consequent to the editing process (FIG. 6(E) and 10(D)). Mice transplanted with AAVS1-edited hHSPCs showed a homogeneous pattern of transgene expression (with approximately 40% positive cells) in all tested tissues. On the contrary, and consistently with the CX3CR1 expression data, transgene expression in mice transplanted with hHSPCs edited at the CX3CR1 locus with the promoter-less cassette was relatively low in the human cells retrieved from the hematopoietic organs of recipient mice (FIG. 6(F)). The highest transgene expression was observed in brain-associated myeloid human cells (FIG. 6(F)). Interestingly, the mean fluorescence intensity (MFI) of the reporters in brain engrafted human cells was higher in mice transplanted with CX3CR1 edited hHSPCs as compared to AAVS1 edited cells, where the expression of the transgene is driven by the PGK promoter (FIG. 6(F)). This data indicates that the promoter-less cassette allows a higher than PGK and specific expression of a targeted integrated transgene under the control of the endogenous CX3CR1 promoter also in vivo, in tissues. Targeting efficiency was evaluated in vivo by performing a ddPCR designed at the vector-to-genome junction region to detect the integrated cassette in tissues collected from the transplanted mice. Targeting efficiency was good in all the groups in hematopoietic organs at sacrifice (FIG. 6(G)). A multilineage editing was confirmed in both myeloid (hCD13) and lymphoid (hCD19) sorted populations from the spleen in all the groups of transplanted mice (FIG. 10(E)), as well as a good targeting efficiency, stable over time, in the peripheral blood of transplant recipients (FIG. 10(F)). This was also proven in the BM of secondary recipients in all the groups, indicating a good efficiency of the editing procedure in the stem compartment (FIG. 10(G)). Interestingly, despite the relatively low human engraftment observed in the brain due to the challenging mouse-into-human setting in the NSG model, a higher targeted integration was retrieved in the brain myeloid progeny of the hHSPCs edited at the CX3CR1 locus, especially with the construct targeting CX3CR1 intron, as compared to the other tissues (FIG. 6(G)). This data suggests an enrichment of the CX3CR1 edited (and haploinsufficient) cells in the brain as compared to hematopoietic organs. Next, an immunofluorescence analysis was performed on brain tissue slices to characterize the brain progeny of the transplanted hHSPCs. Engrafted human cells showed a microglia-like morphology, with a good extent of ramifications, positivity for microglia markers, such as Iba-1 and expression of the transgenes (FIG. 7(A)). To better characterize the morphology of the brain engrafted cells, a branching analysis and a Sholl analysis were performed, using the same Macro employed in the mouse-into-mouse transplantation setting described above. Interestingly, CX3CR1 edited hHSPC progeny cells showed a greater extent of ramifications and in general a more complex morphology and a greater complexity of ramifications as compared to control AAVS1 edited cells (FIG. 7(B-D)). Similarly, the analysis showed that MLCs derived from CX3CR1 edited hHSPC covered a larger surface and displayed more arborizations as compared to control AAVS1 edited cell progeny (FIG. 7(E),(F)). Thus, in vivo differentiation towards a μ-like phenotype of hHSPCs is favored by CX3CR1 haploinsufficiency.

In the above Examples, CX3CR1 was identified as a unique locus that could be exploited to design novel HSC gene therapy approaches endowed with a unique therapeutic potential. Indeed, targeted gene addition at the CX3CR1 locus of HSPCs could result in i) an enhanced capability of the engineered cells and their progeny to engraft in the CNS and replace endogenous microglia with gene modified MLCs upon transplantation, and ii) a robust and regulated expression of the integrated therapeutic transcript in transplant-derived MLCs, with a strong potential of exerting therapeutic effects in the CNS. Haploinsufficiency for CX3CR1 promotes the CNS engraftment of HSPCs upon transplantation as well as their differentiation towards bona fide MLCs. These findings were confirmed across different experimental in vivo settings, namely mouse-into-mouse and human-into-mouse transplants, employing a CX3CR1 haploinsufficient reporter model (Cx3cr1−/+ murine HSPCs) as well as gene edited human HSPCs.

Firstly, murine HSPCs haploinsufficient at the Cx3cr1 locus showed enhanced engraftment and differentiation capability towards MLCs as compared to WT cells in the CNS of transplant recipients. While monitoring the cell engraftment in the CNS of myeloablated recipients receiving Cx3cr1 haploinsufficient murine HSPCs, an early appearance of MLCs was observed with the antigenic features of mature p, rather than of the immature TAμ cells that prevail in early stages post-transplant in WT HSPC transplant recipients [19,28]. Then, in a competitive transplant experiment, Cx3cr1 haploinsufficient murine HSPCs engrafted more robustly in hematopoietic organs and, even more strikingly, in the brain of recipients, as compared to the co-injected WT cells. At morphologic evaluation, the Cx3cr1−/+ cells engrafted in the brain of the recipients displayed morphological features suggestive of a more rapid maturation towards MLCs as compared to their WT counterpart. Complete lack of Cx3cr1 did not increase this phenotype, suggesting the absence of a dosing effect. Similarly, human HSPCs edited at the CX3CR1 locus showed a greater engraftment potential, particularly in the CNS, and a more robust maturation towards bona fide MLCs in a xenotransplant setting, as compared to cells edited at other loci. These data thus indicate both a quantitative and qualitative advantage conferred by CX3CR1 haploinsufficiency to HSPCs in reconstituting the brain myeloid compartment of transplant recipients.

CX3CR1 is one of the main microglia signature genes, active in early microglial precursors, and expressed throughout adulthood [29,30]. Microglia precursors develop into CD45+c-kitlow CX3CR1− immature cells that then mature into CD45+c-kit− CX3CR1+ cells, being CX3CR1 one of the first genes turned on during microglia ontogenesis [31,32]. The CX3CR1 locus could participate as well in the process of microglia repopulation following HSPC transplant, also considering its role in the control of cell proliferation and migration [17] and that perturbations of CX3CR1 expression levels could modulate the process as well. Notably, a similar effect was also observed in hematopoietic organs, especially in the thymus, which was more robustly repopulated by the CX3CR1−/+ or edited HSPCs as compared to WT cells, thus suggesting a possibly wider role of this molecule in the hematopoietic reconstitution process.

In the above Examples, transcriptomic analysis performed on MLCs sorted from competitively transplanted mice revealed upregulation of pathways associated to microglia differentiation and migration, including Cdc42 associated genes, in a cell cluster enriched in Cx3cr1−/+ cells. Cx3cr1-induced signal transduction pathway can activate Cdc42 via Syk and PI3K that are ultimately required for macrophage chemotaxis towards Cx3cl1 [33]. Cdc42 signal transduction pathway has been also associated with myelopoiesis and HSC engraftment as dysregulation of Cdc42 results in disorganized actin structure in hematopoietic cells and defective engraftment in stem cell transplant protocols [34,35]. Indeed, HSPCs from Cdc42−/− mice show defective migration and adhesion, which is associated with abnormal F-actin assembly, homing, and engraftment/retention in the bone marrow [36]. Thus, Cx3cr1 haploinsufficiency could perturb Cdc42 associated pathways and ultimately result in cytoskeleton rearrangements, increased cell motility and cell cycle progression during the microglia reconstitution process following HSPC transplant.

This genomic locus could be exploited to enhance the ability of HSPCs to engraft and repopulate the hematopoietic system and, more obviously, the CNS myeloid compartment of transplant recipients. Moreover, since CX3CR1 is robustly expressed by microglia in neuropathological conditions up to being considered as a possible relevant therapeutic target in neurodegenerative disorders [37], a CX3CR1 gene editing and targeted gene addition approach was designed in HSPCs in the above Examples that could also allow obtaining a regulated and robust therapeutic transcript expression by transplant-derived MLCs. To challenge the applicability of this strategy, new tools were developed for CRISPR/Cas9-based gene editing and AAV6 based gene addition at the human CX3CR1 locus allowing for the generation of a population of edited CX3CR1 haploinsufficient/knocked out HSPCs that could express a therapeutic transcript under the control of the CX3CR1 promoter. As control, the AAVS1 locus was chosen, a well-validated safe harbour for hosting DNA transgenes with open chromatin structure and no known adverse effects resulting from the inserted DNA fragment of interest [26].

The use of a promoterless, splice-trapping cassette encoding for a fluorescent reporter to be inserted at the CX3CR1 locus in a CX3CR1-expressing cell line and in hHSPCs. Targeted insertion at the chosen intronic region of the CX3CR1-expressing cell line resulted in a regulated expression of the reporter gene and concomitant CX3CR1 knock-out only in HDR-edited cells, while NHEJ-INDELs did not impact CX3CR1 expression. Since only a minor fraction of the treated HSPCs will contain biallelic integrations, this strategy will mostly result in the generation of the desired haploinsufficient cells. These results were confirmed on hHSPCs, where integration of the reporter cassette resulted in a dim, but consistent expression of the fluorescent reporters, reliably reflecting the activity of the CX3CR1 promoter in this heterogenous cell population when cultured in vitro. Molecular analysis confirmed a good efficiency of targeted integration, resulting in CX3CR1 transcriptional and protein downregulation, reproducing a haploinsufficiency condition. Interestingly, as mentioned above, the CX3CR1 edited hHSPC showed a higher capability of engraftment into myeloablated immunodeficient recipients as compared to the control AAVS1 edited hHSPCs. These findings were also maintained in secondary transplant recipients, indicating that CX3CR1 editing occurred in long-term repopulating HSCs and that its modulation could impact the functional properties of these cells and of their progeny. Remarkably, a higher targeted integration was retrieved in the brain myeloid progeny of the transplanted CX3CR1-edited hHSPCs as compared to what was observed in the cells that repopulated the hematopoietic organs of the primary and secondary recipients. This finding suggests an enrichment of the edited cells within the CNS-engrafted progeny of the transplanted HSPCs, likely due to an advantage in engraftment in the CNS of the CX3CR1-edited cells. This enrichment was not seen in the AAVS1 group, thus indicating in CX3CR1 down-regulation the most likely mechanism leading to this enrichment. Of note, the branching study revealed that the CX3CR1-edited cells engrafted in the brain more robustly differentiated towards human MLCs and acquired a more mature morphology as compared to the safe harbor-edited MLCs, thus confirming that the reduction of CX3CR1 expression also in this humanized setting could favor the maturation and differentiation of the transplanted HSPCs and their progeny in the CNS towards microglia.

Importantly, the targeted integration of a promoterless cassette at the CX3CR1 locus of HSPCs allowed obtaining specific, regulated, and robust transgene expression in their progeny engrafted in hematopoietic organs and in the CNS. Indeed, the transgene integrated at the CX3CR1 locus was expressed consistently with the activity of the endogenous promoter, with substantially higher expression in CNS-associated transplant progeny MLCs as compared to similarly edited cells engrafted in hematopoietic organs and to cells edited at the AAVS1 locus, where the integrated cassette contained a conventional PGK promoter to drive transgene expression. The latter finding is of particular relevance as the PGK promoter is currently used in HSPC gene therapy clinical applications for neurodegenerative/neurometabolic conditions [1,2,4,5]: the robust, lineage specific and regulated therapeutic transcript expression coupled to the faster pace of transplant progeny cell engraftment in the CNS that can be achieved with CX3CR1 editing and targeted gene addition approach could be particularly advantageous in these conditions. Indeed, despite HSC gene therapy has shown unprecedented clinical benefits in neurometabolic patients treated at a very early disease stage or when pre-symptomatic, symptomatic patients cannot benefit from the treatment because of the progression of their disease before therapeutic cells could exert their effects in the CNS. The approach provided herein, by shortening and fostering the microglia reconstitution process after engineered HSPC transplantation, could expand the temporal window for treatment and guarantee robust therapeutic gene expression. Finally, the development of a novel, simple, efficient and very specific treatment platform for NMDs based on an empowered HSC-targeted gene addition could reduce potential safety concerns associated with side effects related to vector integration [8-11], as high sensitivity safety workflows are being quickly developed and applied prior to clinical translation, therefore maximizing the chances for successful clinical implementation and long-term safety of site-specific genome editing therapies [38].

In conclusion, based on indications strongly supporting a quantitative and qualitative advantage in the post-transplant maturation of CX3CR1 deficient HSPCs towards MLCs, a promoterless, splice trapping cassette was designed allowing for efficient editing and targeted gene addition at the CX3CR1 locus, enhancing the ability of HSPCs to engraft and repopulate the hematopoietic system and the CNS myeloid compartments of transplant recipients, obtaining specific, regulated and robust transgene expression in the hematopoietic system and in the CNS. By fostering CNS myeloid repopulation following HSPC transplant and anticipating the clinical benefit associated with HSPC gene therapy, this platform is to be applied for the treatment of disorders characterised by a rapid progression of neurodegeneration where CX3CR1 edited and engineered cells could express at high levels a therapeutic transcript in the myeloid progeny of brain engrafted cells, thus allowing the development of new and more effective and specific platform for the treatment of neurometabolic and neurodegenerative diseases.

### Materials and Methods

The following materials and methods were employed in the above examples.

TABLE 1
sgRNAs targeting exon 5 (left) and intron 4
(right) of human CX3CR1 locus.

| sgRNA targeting | sgRNA targeting |
|---|---|
| CX3CR1 exon 5 | CX3CR1 intron 4 |
| sgRNA1: CUGGCCC | SgRNA5: ACUAUAGG |
| GUGCUCCGCAAUG | GCUGGUAAUCGU |
| (SEQ ID NO: 12) | (SEQ ID NO: 2) |
| SgRNA2: UGACGC | SgRNA6: AAAGCUCA |
| CAUGCUGCACGGUC | CGCUUUAACCGA |
| (SEQ ID NO: 13) | (SEQ ID NO: 14) |
| SgRNA3: CACGAUG | SgRNA7: GACCCAUC |
| UCCCCAAUAUAAC | UGUGGUAUCACG |
| (SEQ ID NO: 15) | (SEQ ID NO: 16) |
| SgRNA4: GUAAAUG | SgRNA8: AUGGGGCU |
| UCGGUGACACUCU | CCGUUAGUCUGC |
| (SEQ ID NO: 17) | (SEQ ID NO: 18) |
| SgRNA9: UGAUUCA | |
| GGGAACUGAUCCA | |
| (SEQ ID NO: 1) | |

TABLE 2

| | |
|---|---|
| AAV donor | gctgaggcggaaggataatctgaggtcaggagtttgagaccag |
| templates | cctggctaacatgatgaaaccccatctctactaaaaatacaaa |
| sequences | aattagccaggcgtggtagtgcacgcctataatcccagctatt |
| promoterless. | tgggaggctgaggcaggagaatcgcttgaaccccaggggacag |
| YFP_SA-CX exon | aggttgcagtgagctgagatcgcaccactgcactccagcctgg |
| | gtgacacagcgagactccatttaaaaaaaaaaaaatgcctaga |
| | gccaaatgctcacagagccatttactgcatggctttgggcaag |
| | tcaaaggagtccgcctctcctgtcagaagagtctgttgcagtc |
| | ttcatcacaagactgttgtggggattaaacaagatggcaagtg |
| | ggaagttgggaaatgtagtgtgcacccaaccaatatttgtttc |
| | ttcctgcctgcctacatatgaggccacacagaattccaacttt |
| | gtttctctgataactaacacagttacttgtttttctttctgat |
| | ccagGCCTTCACcatggAACTGACCTCTTCTCTTCCTCCCACA |
| | GGCCGCCAccatggtgagcaagggcgaggagctgttcaccggg |
| | gtggtgcccatcctggtcgagctggacggcgacgtaaacggcc |
| | acaagttcagcgtgtccggcgagggcgagggcgatgccaccta |
| | cggcaagctgaccctgaagttcatctgcaccaccggcaagctg |
| | cccgtgccctggcccaccctcgtgaccaccttcggctacggcc |
| | tgatgtgcttcgcccgctaccccgaccacatgaagcagcacga |
| | cttcttcaagtccgccatgcccgaaggctacgtccaggagcgc |
| | accatcttcttcaaggacgacggcaactacaagacccgcgccg |
| | aggtgaagttcgagggcgacaccctggtgaaccgcatcgagct |
| | gaagggcatcgacttcaaggaggacggcaacatcctggggcac |
| | aagctggagtacaactacaacagccacaacgtctatatcatgg |
| | ccgacaagcagaagaacggcatcaaggtgaacttcaagatccg |
| | ccacaacatcgaggacggcagcgtgcagctcgccgaccactac |
| | cagcagaacacccccatcggcgacggccccgtgctgctgcccg |
| | acaaccactacctgagctaccagtccaagctgagcaaagaccc |
| | caacgagaagcgcgatcacatggtcctgctggagttcgtgacc |
| | gccgccgggatcactctcggcatggacgagctgtacaagtaac |
| | TCGAGCCGCTGAtcagcctcgactgtgccttctagttgccagc |
| | catctgttgtttgcccctcccccgtgccttccttgaccctgga |
| | aggtgccactcccactgtcctttcctaataaaatgaggaaatt |
| | gcatcgcattgtctgagtaggtgtcattctattctggggggtg |
| | gggtggggcaggacagcaagggggaggattgggaagacaatag |
| | caggcatgctggggatgcggtgggctactagtATCAGTTCCCT |
| | GAATCAGTGACAGAAAACTTTGAGTACGATGATTTGGCTGAGG |
| | CCTGTTATATTGGGGACATCGTGGTCTTTGGGACTGTGTTCCT |
| | GTCCATATTCTACTCCGTCATCTTTGCCATTGGCCTGGTGGGA |
| | AATTTGTTGGTAGTGTTTGCCCTCACCAACAGCAAGAAGCCCA |
| | AGAGTGTCACCGACATTTACCTCCTGAACCTGGCCTTGTCTGA |
| | TCTGCTGTTTGTAGCCACTTTGCCCTTCTGGACTCACTATTTG |
| | ATAAATGAAAAGGGCCTCCACAATGCCATGTGCAAATTCACTA |
| | CCGCCTTCTTCTTCATCGGCTTTTTTGGAAGCATATTCTTCAT |
| | CACCGTCATCAGCATTGATAGGTACCTGGCCATCGTCCTGGCC |
| | GCCAACTCCATGAACAACCGGACCGTGCAGCATGGCGTCACCA |
| | TCAGCCTAGGCGTCTGGGCAGCAGCCATTTTGGTGGCAGCACC |
| | CCAGTTCATGTTCACA (SEQ ID NO: 19) |
| promoterless. | GGCCGCTGAGAACTCATCACAGCCTGAGACAGCTTAGGTTGTA |
| YFP_SA-CX | AAGCCCCGGCCCACTTATCCCAGGAGAGTCTGGGTGAGATGCA |
| intron | GGCCCCAAAGCAGAGGCTGGGAAGCGAGAAGTGACACACCCTG |
| | GCTGGGTGGGCCCTCATCTTGGTGAGACACCACCTGGGTAAAA |
| | CCATCATGGAAAGGGTGTAGTGGGGCGTGGAAACTCCCTCGGT |
| | TAAAGCGTGAGCTTTGCTGTAAGTTGTGGTAAGGAGGGAGGCA |
| | GTGACAACCAGGAGGCCTGTTTTGAGGGTTTCTGAGGGACCCA |
| | TCTGTGGTATCACGAGGAGACGCCCAGAGGAGCCGTGTGAAAG |
| | GGCTGCCTCCCAGCCGGCTCTGGAGTGAATGAGCAGCAAGTCC |
| | TGGCTGCGAAAAGAAGGGGAGTGCAGCCTGCAGAAGTGTCTTC |
| | TTTTTTCAATTCCTGCTCAGAAGGAAACAGGAGATAAGAATAG |
| | TGGGGAAGTCCAAACCAAAGTGAACTATAGGGCTGGTAATTCT |
| | AGATTcgAACTGACCTCTTCTCTTCCTCCCACAGGCCGCcaCC |
| | Atggtgagcaagggcgaggagctgttcaccggggtggtgccca |
| | tcctggtcgagctggacggcgacgtaaacggccacaagttcag |
| | cgtgtccggcgagggcgagggcgatgccacctacggcaagctg |
| | accctgaagttcatctgcaccaccggcaagctgcccgtgccct |
| | ggcccaccctcgtgaccaccttcggctacggcctgatgtgctt |
| | cgcccgctaccccgaccacatgaagcagcacgacttcttcaag |
| | tccgccatgcccgaaggctacgtccaggagcgcaccatcttct |
| | tcaaggacgacggcaactacaagacccgcgccgaggtgaagtt |
| | cgagggcgacaccctggtgaaccgcatcgagctgaagggcatc |
| | gacttcaaggaggacggcaacatcctggggcacaagctggagt |
| | acaactacaacagccacaacgtctatatcatggccgacaagca |
| | gaagaacggcatcaaggtgaacttcaagatccgccacaacatc |
| | gaggacggcagcgtgcagctcgccgaccactaccagcagaaca |
| | cccccatcggcgacggccccgtgctgctgcccgacaaccacta |
| | cctgagctaccagtccaagctgagcaaagaccccaacgagaag |
| | cgcgatcacatggtcctgctggagttcgtgaccgccgccggga |
| | tcactctcggcatggacgagctgtacaagtaacTCGAGCCGCT |
| | GAtcagcctcgactgtgccttctagttgccagccatctgttgt |
| | ttgcccctcccccgtgccttccttgaccctggaaggtgccact |
| | cccactgtcctttcctaataaaatgaggaaattgcatcgcatt |
| | gtctgagtaggtgtcattctattctggggggtggggtggggca |
| | ggacagcaagggggaggattgggaagacaatagcaggcatgct |
| | ggggatgcggtgggctaCTAGTCGTAGGGGGAATTAGTCACCC |
| | GGAGACTAGCCCAGCAGACTAACGGAGCCCCATCCTCCATCTT |
| | GAATCAGTCAGCCCCTCTATGACTGCAGAGTCCTGAATGATGG |
| | CAACACCTTCTCTTCACTTAGGGTTGTAGGATGACCAACAGTC |
| | CTGATTTGCCTGGGACTGAGGGGTTCCCAATAGATGGGACTTT |
| | CAGGGCTAAAACCAGGAAAGTCCTGGGCAGCCCAAAACAAGGT |
| | AGTCACTCTAGAGTGTATGACTCTGTCTGATACCTGCTAAGAA |
| | AGAGAAGGACTTGTTGATTATAAGGAGAAGAGGAGGTGAAATG |
| | GTTCTCAAAAAACAAAGATGAGGGCTTCCGGGTGCTGTTCTGC |
| | CCAAGGCTCTGGGTCTGAGGCTTCTCTCTCCAGGCCTAGCTTC |
| | ATGGAAAAGTAAGGGGCCAGAGGGTGGAAAAGGTGGAAACAAA |
| | GGAAGAGGATGGAGAATTGCTTTGGGGAAGTTTGGACTGGAAG |
| | TGTGAATTACAGCTGC (SEQ ID NO: 20) |

Sequences of AAV donor templates (from left homology arm to right homology arm):
promoterless.YFP_SA—CX exon: splice trapping integration cassette encoding for YFP reporter with HA targeting sgRNA9 cutting site;
promoterless.YFP_SA—CX intron: splice trapping integration cassette encoding for YFP reporter with HA targeting sgRNA5 cutting site;

TABLE 3
CX3CR1 Integrated Cassette Sequences

| | |
|---|---|
| CX3CR1- | CTGACCTCTTCTCTTCCTCCCACAGGCCGCcaCCAtggtgagcaagggcga |
| Intron | ggagctgttcaccggggtggtgcccatcctggtcgagctggacggcgacgt |
| Integrated | aaacggccacaagttcagcgtgtccggcgagggcgagggcgatgccaccta |
| Cassette | cggcaagctgaccctgaagttcatctgcaccaccggcaagctgcccgtgcc |
| | ctggcccaccctcgtgaccaccttcggctacggcctgatgtgcttcgcccg |
| | ctaccccgaccacatgaagcagcacgacttcttcaagtccgccatgcccga |
| | aggctacgtccaggagcgcaccatcttcttcaaggacgacggcaactacaa |
| | gacccgcgccgaggtgaagttcgagggcgacaccctggtgaaccgcatcga |
| | gctgaagggcatcgacttcaaggaggacggcaacatcctggggcacaagct |
| | ggagtacaactacaacagccacaacgtctatatcatggccgacaagcagaa |
| | gaacggcatcaaggtgaacttcaagatccgccacaacatcgaggacggcag |
| | cgtgcagctcgccgaccactaccagcagaacacccccatcggcgacggccc |
| | cgtgctgctgcccgacaaccactacctgagctaccagtccaagctgagcaa |
| | agaccccaacgagaagcgcgatcacatggtcctgctggagttcgtgaccgc |
| | cgccgggatcactctcggcatggacgagctgtacaag |
| | (SEQ ID NO: 21) |
| CX3CR1- | CTGACCTCTTCTCTTCCTCCCACAGGCCGCCAccatggtgagcaagggcga |
| Exon | ggagctgttcaccggggtggtgcccatcctggtcgagctggacggcgacgt |
| Integrated | aaacggccacaagttcagcgtgtccggcgagggcgagggcgatgccaccta |
| Cassette | cggcaagctgaccctgaagttcatctgcaccaccggcaagctgcccgtgcc |
| | ctggcccaccctcgtgaccaccttcggctacggcctgatgtgcttcgcccg |
| | ctaccccgaccacatgaagcagcacgacttcttcaagtccgccatgcccga |
| | aggctacgtccaggagcgcaccatcttcttcaaggacgacggcaactacaa |
| | gacccgcgccgaggtgaagttcgafatcgacttcaaggaggacggcaacat |
| | cctggggcacaagctggagtacaactacaacagccacaacgtctatatcat |
| | ggccgacaagcagaagaacggcatcaaggtgaacttcaagatccgccacaa |
| | catcgaggacggcagcgtgcagctcgccgaccactaccagcagaacacccc |
| | catcggcgacggccccgtgctgctgcccgacaaccactacctgagctacca |
| | gtccaagctgagcaaagaccccaacgagaagcgcgatcacatggtcctgct |
| | ggagttcgtgaccgccgccgggatcactctcggcatggacgagctgtacaa |
| | g (SEQ ID NO: 22) |

*Splice Acceptor sequence in Bold font; linker sequence in Italics; yellow fluorescent protein (YFP) sequence in plain lower case font

### Mouse Studies

All experiments and procedures involving animals were performed with the approval of the Dana Farber Animal Facility Institutional Animal Care and Use Committee (IACUC 15-031). Wild-type C57BL/6J mice or C57BL6/Ly5.1 mice (hereafter called CD45.2 or CD45.1 respectively) were obtained from Jackson Lab or Charles River. NOD-SCID-IL2Rg−/−(NSG) female mice (Stock No: 005557) were obtained from The Jackson Laboratory.

Cx3cr1GFP/+(referred as Cx3cr1−/+) mice were generated by crossing Cx3cr1GFP/GFP (referred as Cx3cr1−/−) obtained from The Jackson Laboratory (Stock. No 005582) with wild type CD45.2 mice (referred as Cx3cr1+/+). For all transplantation experiments, mice were randomly distributed to each experimental group.

### Isolation, Transduction and Transplantation of Murine Hematopoietic Cells

Seven/eight-week-old wild type, Cx3cr1−/+ or Cx3cr1−/− mice were euthanized with CO2, and the BM was harvested by crushing bones. After BM lysis, HSPCs were purified by Lin− selection using the Lineage Cell Depletion Kit (Miltenyi, #130-090-858) with the autoMACS™ magnetic separation, following manufacturer's instruction.

### Sorting Experiments

GFP+ and GFP− cells were sorted from the Lin− pool isolated from CCx3cr1−/+ mice using the BD FACSAria II high speed cell sorter. Collected cells were freshly transplanted IV into busulfan (4 doses 25 mg/kg) conditioned CD45.1 recipients at a 1:1 donor/recipient ratio. Mice also received 1.0*106 CD45.1 BMNC IV 5 days post-transplant for hematopoietic rescue. 45 days post-transplant mice were sacrificed, and BM and brain were collected for cytofluorimetric analysis.

### Standard Transplantation Experiments

Isolated Cx3cr1+/+ or Cx3cr1−/+ Lin− were transplanted IV into busulfan conditioned CD45.1 recipients (1.0*106/mouse) after 12-16 h of culture in StemSpan medium supplemented with cytokines as previously described [39]. Mice were sacrificed at 45, 90, 180 days post-transplant to collect hematopoietic organs and brain for flowcytometric analysis.

### Competitive Experiments

Isolated Cx3cr1+/+, Cx3cr1−/+ and Cx3cr1−/− Lin− were transduced with LVs, for 12-16 hours at Multiplicity of Infection (MOI) 100 [39]. The following LVs were used: pCCLsin.cPPT.humanPGK.BlueFluorescentProtein.Wpre (BFP-LV) for Cx3cr1−/+ or Cx3cr1−/− HSPCs and pCCLsin.cPPT.humanPGK.mCherryProtein.Wpre (mCherry-LV) for Cx3cr1+/+ HSPCs. A fraction of the transduced cells was cultured for 10 days in vitro [39] to assess transgene expression by cytofluorimetric analysis. Transduced cells were injected via the tail vein or directly in the CNS by means of ICV injection into seven/eight-week-old conditioned CD45.1 female mice as previously described [21].

For IV group a total of 1.0*106 cells/mouse (0.5*106 Cx3cr1−/+ or Cx3cr1−/− BFP+ HSPCs+0.5*106 Cx3cr1+/+ mCherry+ HSPCs) was injected. For ICV injection, a total of 0.3*106 cells/mouse (0.15*106 Cx3cr1−/+ BFP+ HSPCs+0.15*106 Cx3cr1+/+ mCherry+ HSPCs) was injected. ICV transplanted mice received also 1.0*106 CD45.1 BMNC IV 5 day post-transplant for hematopoietic rescue.

### Cell Lines and Primary Cell Culture

HEK293T cells used for AAV and LV productions were cultured in Dulbecco's Modification of Eagle's Medium (DMEM) 4.5 g/L glucose or Iscove's modified Dulbecco's medium (IMDM; Corning), respectively, supplemented with 10% heat-inactivated fetal bovine serum (FBS), 100 IU/ml penicillin, 100 μg/ml streptomycin and 2% glutamine. To choose and test the tools for gene editing at the CX3CR1 locus, RPMI 8226 (ATCC #CCL-155™) was used, a suitable transfection host cell line expressing CX3CR1 gene, and K562 (ATCC #CCL-243™), not expressing CX3CR1, as negative control. RPMI 8226 were cultured in RPMI medium supplemented with 10% FBS, 100 IU/ml penicillin, 100 μg/ml streptomycin and 2% glutamine. K562 were cultured in Iscove's modified Dulbecco's medium (IMDM; Corning) supplemented with 10% FBS, 100 IU/ml penicillin, 100 μg/ml streptomycin and 2% glutamine. Both cell lines were cultured in a 5% CO2 humidified atmosphere at 37° C.

Commercial cord blood CD34+ human HSPCs were purchased frozen from AllCells and were thawed using Thymocyte Thawing Media (TTM) medium, modified from [40]. TMM medium was prepared with RMPI medium supplemented with 30% FBS, 1% Pen/Strep, 10 μg/ml DNase I (Sigma), 20 U/ml heparin. Cord blood CD34+ cells were gently thawed in pre-warmed TMM medium and left in the water bath at 37 C for 1 hour. Cells were then spun down (300 rcf 5 minutes) and seeded at the concentration of 5×105 cells/ml in a cytokine-enriched medium for prestimulation. Serum-free StemSpan SFEM II medium (StemCell Technologies, #09605) was supplemented with 100 IU/ml penicillin, 100 μg/ml streptomycin, 100 ng/ml hSCF (PeproTech), 100 ng/ml hFlt3-L (PeproTech), hTPO 100 ng/mL (PeproTech), hIL-6 (PeproTech) and SRI (StemRegenin1; 0.75 μM, CellagenTech). Cells were left in pre-stimulation for two days before the editing procedure. Cord blood CD34+ cells were cultured in a 5% CO2 humidified atmosphere at 37° C. in low oxygen conditions.

### LV and AAV6 Production

Third-generation SIN Lentiviral vectors were produced and titered as previously described [39]. For in vitro experiments, AAV6 vectors were produced using the AAVpro® Purification Kit (Takara Bio #6666) according to manufacturer instructions and titered as previously described [41]. For in vivo experiments, AAV6 vectors were produced using iodixanol gradients and ultracentrifugation by the Boston Children's viral vector core.

### CRISPR/Cas9 Gene Editing in Cell Lines and in Human Hematopoietic Stem Cells

Sequences of the gRNAs were designed using an online tool [42] and selected for predicted specificity score and on-target activity. AAV6 donor templates were generated from a construct containing AAV2 inverted terminal repeats (ITRs) as previously reported [26]. Vector maps were designed with SnapGene software v5.0.7 (from GSL Biotech; available at snapgene.com).

For experiments in cell lines, RNP complexes were assembled by incubating at 1:1.5 molar ratio Streptococcus pyogenes (Sp)Cas9 protein (Integrated DNATechnologies—IDT) with pre-annealed synthetic Alt-R® crRNA:tracrRNA (IDT) for 15′ at room temperature together with 0.1 nmol of Alt-R® Cas9 Electroporation Enhancer (IDT), added prior to electroporation according to manufacturer's instructions. Both RPMI 8226 and K562 were nucleofected (FF120 program, Lonza 4D-Nucleofector) with the SF Cell 4d Nucleo Kit (Lonza #V4XC-2032) using 50 μM of the different sgRNAs combined with CRISPR/Cas9 in the form of RNP complexes. AAV6 transduction (20000 MOI) was performed right after electroporation, maintaining the cells at a density of 0.25×106 cells/ml. After 5 h, cells were diluted in the proper medium and moved to a bigger well to maintain a cell density of 0.5-0.7×106 cells/ml overnight. The following day (approximately 12-16 hours after) cells were washed with PBS and seeded for expansion for molecular characterization.

For experiments in primary hematopoietic stem cells (CD34), RNP complexes were assembled by incubating at RT for 5′ (Sp)Cas9 protein protein (IDT) with synthetic single guide RNAs (sgRNA) chemically modified (with 2′-O-Methyl at 3 first and last bases, 3′ phosphorothioate bonds between first 3 and last 2 bases and addition of 80-mer SpCas9 scaffold to create a single guide RNA) obtained from Synthego. Per 300,000-500,000 cells in in vitro experiments, nucleofection strips from LONZA Kit S (P3 Primary Cell 4D-Nucleofector™ X #V4XP-3032) were used, mixing 6 μg of Cas9 protein with 3,2 μg of sgRNA at room temperature for 5′. For in vivo experiments, the Lonza 4D nucleocuvette cuvettes from LONZA Kit L (P3 Primary Cell 4D-Nucleofector™ X #V4XP-3012) were used, scaling up the amounts of cells and reagents of five folds.

After thawing, cells were put in pre-stimulation for 2 days in the cytokine-enriched medium. Cells were then washed with PBS, counted and nucleofected with the assembled RNP complexes (DZ100 program, Lonza 4D-Nucleofector). AAV6 transduction was performed right after electroporation at 20K MOI, maintaining the cells at a density of 0.25×106 cells/ml. After 5 h, cells were diluted with the cytokine-enriched medium and moved to a bigger well to maintain a cell density of 0.5-0.7×106 cells/ml overnight. The following day (approximately 12-16 hours after) cells were washed with PBS and seeded for expansion for flow cytometric analysis and molecular characterization or were prepared for transplantation.

### Molecular Characterization of Edited Cells

### TIDE Analysis

4-5 days after electroporation, a fraction of cells (approximately 105) was collected. DNA was extracted using DNA Extraction QE Buffer (Biosearc Technologies #QE09050) and amplified using different primer assays according to the target genomic region. A list of primers and thermal protocols is provided herein. After amplification, the PCR product was run on 1.5% Agarose gel (Tte Laboratories Inc., #BMAG01) and DNA was extracted from the gel with GeneJET Gel Extraction Kit (Life Technologies, #K0692). Purified DNA was then sequenced (SANGER seq) to evaluate the percentage of Insertions and Deletions (INDELs) with the TIDE (Tracking of Indels by Indels by DEcomposition) software (Brinkman et al., 2014).

### Targeted Integration (HDR)

To evaluate the efficiency of integration by homology direct repair (HDR), DNA was extracted with the QIAamp DNA Micro Kit (QIAGEN) from cells expanded in vitro for 10-14 days. 20-50 ng of genomic DNA were analyzed using the QX200 Droplet Digital PCR System (Bio-Rad) according to the manufacturer's instructions. Different assays of primers and probes were designed on the junction between the vector sequence and the targeted locus. Human albumin was used for normalization. A list of primers and thermal protocols is provided herein.

### Gene Expression

To evaluate the expression of CX3CR1 gene, RNA was extracted with RNeasy Plus Micro Kit (Qiagen) from cells expanded in vitro for 7-10 days. cDNA was synthetized with Maxima Reverse Transcriptase (ThermoFisher #EP0742) and CX3CR1 expression was quantified using TaqMan® Assays Hs01922583_s1 and human GAPDH Hs02786624_g1 as housekeeping reference. 1 ng RNA equivalent was analyzed using the QX200 Droplet Digital PCR System (Bio-Rad). Poisson statistical analysis of the numbers of positive and negative droplets yields absolute quantification of the target sequence.

### Flow Cytometry

CX3CR1 Monoclonal Antibody (2A9-1) eBioscience™ was used to assess protein expression by flow cytometry in cells expanded in vitro for 7 days. Cells were collected and resuspended in 100 ul of MACS buffer with 2:100 FcR Blocking Reagent (Miltenyi, #130-059-901) and incubated 10′ at 4 C to avoid a specific binding of the antibodies. Cells were then incubated with the antibody for 20′ (labeling procedure) at 4 C. After washing, cells from different tissues were resuspended in MACS buffer (300-400 μl). Vital dye (7AAD) was added, and samples were analysed at BD LSR Fortessa. Results were analysed by FlowJo 10.8.0 software.

### Transplantation of Edited Human Hematopoietic Stem Cells in Immunodeficient Recipients

Edited human HSPCs were transplanted into 7-8 weeks old NSG females conditioned with busulfan (16.25 mg/kg per 4 days). Cells were administered IV (0.5*106/mouse) and ICV (0.3*106/mouse) as previously described [21]. Mice were then provided with syngeneic BMNC for hematopoietic rescue 5 days post-transplant.

Secondary transplantation was performed upon injection of 2×106 beads-purified human CD34+ cells (CD34 MicroBead Kit, Miltenyi #130-046-702) harvested from the BM of primary engrafted NSG mice euthanised at 12 weeks post-transplant. Mice were then provided with syngeneic BMNC for hematopoietic rescue 5 days post-transplant. Transplanted mice were monitored by bleeding once/month and euthanised after 12 weeks as previously described.

### Evaluation of Peripheral Engraftment in the Blood

Human CD45+ cell engraftment and the presence of edited cells were evaluated by bleeding mice periodically from the tail vein. 0.1 ml of blood was obtained per sample and collected into tubes containing 10 μl EDTA solution (45 ng/ml) to avoid coagulation of the blood during the blood draw. Cells of donor origin were detected by FACS analysis after lysis of erythrocytes with ACK (10 minutes at room temperature) and specific staining as specified below. Remaining cells were used to extract DNA with QIAamp DNA Micro Kit (QIAGEN) to perform molecular analysis as specified below. A list of flow cytometry antibodies is provided herein.

### Mouse Tissue Collection and Processing for Flow Cytometry and Histology

According to the time and the experimental settings mice were euthanized under deep anaesthesia (Ketamine/Xylazine mix) by extensive intracardiac perfusion with cold PBS for 15 minutes after clumping the femur. Hematopoietic organs and brain were then collected and differentially processed.

BM cells were harvested by flushing the tibias and femurs with PBS 2% FBS. Spleen and thymus were mechanically disaggregated on a cell strainer (40 μm) in PBS 2% FBS (15 ml for spleen, 5 ml for thymus). 500 μl of homogenate tissues were centrifuged at 900 rcf for 5′ and then re-suspended in 100 μl of blocking solution (MACS buffer with 2:100 FcR Blocking Reagent Miltenyi #130-059-901 and 1:100 CD16/CD32 Blocking Assay, BD Biosciences #553142) and incubated 10 min at 4 C to avoid a specific binding of antibodies. Cells were then incubated with specific antibodies for 20 min (labeling procedure) at 4 C. After washing, cells from different tissues were resuspended in MACS buffer (300-400 μl). Vital dye (DAPI or 7AAD) was added, and samples were analysed at BD LSR Fortessa. Results were analysed by FlowJo 10.8.0 software. A list of flow cytometry antibodies is provided herein.

Brain was removed and the two hemispheres were differently processed. For immunofluorescence analysis, one hemisphere was fixed for 24 hours in 4% PFA, embedded in OCT compound and stored at −80° C., after equilibration in sucrose gradients (from 10 to 30%) supplemented with 0.02% of sodium azide to avoid contamination. For flow cytometry analysis, cells were obtained by mechanic disaggregation of one brain hemisphere in 1.5 ml in EBSS medium and processed with papain-based digestion procedure according to the Neural Tissue Dissociation Kit (Miltenyi, #130-092-628). After washing with EBSS medium, the digested suspension was enriched in myeloid cells with Percoll gradient (700 g for 15 minutes, no break). Cell suspension was then washed with PBS 2% FBS, put in blocking solution and stained with specific antibodies as described above for flow cytometric analysis. For the ICV experiment, the brain hemisphere containing the injection site was analysed by FACS analysis, while the contralateral part was used for immunofluorescence analysis.

From all the tissues, a fraction of the cell suspension was stored at −80 C. DNA was then extracted with the QIAamp DNA Micro Kit (QIAGEN) to perform molecular characterization and evaluate editing efficiency in vivo as described above. A list of primers and thermal protocols is provided herein.

### Immunofluorescence Analysis

Brains embedded in OCT were serially cut in the sagittal planes on a cryostat in 18 μm sections. Brain sections were obtained from the contralateral side of cell injection for intracerebroventricularly transplanted mice.

Tissue slides were washed twice with PBS, air dried and blocked with 0.3% Triton, 10% FBS for 1 hour at room temperature. Then sections were incubated over night at 4 C with primary antibodies diluted in PBS, 0.3% Triton, 10% FBS as follows: rabbit anti Iba1 (Wako) 1:250; chicken anti-GFP (Abcam) 1:100; rabbit anti-cherry (Abcam) 1:100; anti-human nuclei (Sigma Aldrich) 1:100. The secondary antibodies goat IgG anti-Chicken Alexa Fluor 488, goat IgG anti-Rabbit Alexa Fluor 488, 546 or 633, goat IgG anti-Rat Alexa Fluor 546 or 633, goat IgG anti-Mouse Alexa Fluor 546 (Molecular Probes, Invitrogen) were diluted 1:500 in PBS, 1% FBS and incubated with sections for 90 minutes at room temperature. Nuclei were stained with DAPI (Roche) 1:30 in PBS. Slices were washed in PBS, air dried and mounted with Fluorsafe Reagent (Calbiochem). Not transplanted mice were used as negative controls for the reporter transgene staining. Incubation with secondary antibody alone was performed to exclude the background signal. Samples were analyzed with a confocal microscope (Zeiss and Leica TCS SP2; Leica Microsystems Radiance 2100; Bio-Rad; Leica SPE confocal) (k excitation=488,586, 660).

Fluorescent signal was processed by Lasersharp 2000 software. Images were imported into ImageJ software and processed by using automated level correction. For the reconstruction of brain sections, we used a fluorescence microscope Delta Vision Olympus Ix70 for the acquisition of the images, which were then processed by the Soft Work 3.5.0 software. Images were then imported into the Adobe Photoshop CS 8.0 software and reconstructed.

For branching analysis, a Macro was developed in the lab with ImageJ software to perform a standardized analysis on multiple individual cells (>50 per group) from different transplanted mice (n=3/group). Branching results obtained on fluorescent markers were confirmed on Iba-1stained cells, independently from the fluorescent signal reporter.

Automated Sholl analysis [22] was performed by applying the same strategy, to study the radial distribution of microglia branches around the cell body. The sum intersections and number of intersection radii were selected as parameters to characterize donor derived cells, in terms of complexity and spatial extension of the cell arborizations, respectively.

Cell Sorting from Hematopoietic Organs of Transplanted NSG Mice

To evaluate gene editing efficiency of the hematopoietic compartment in vivo, engrafted cells were sorted from the spleen of transplanted NSG mice as previously described [44]. Briefly, spleens were crushed, and cell suspension was filtered with a 40-μm cell strainer with cold MACS buffer. The homogenate was then lysed with ACK lysis buffer. After washing with MACS buffer, cells were stained with the dedicated anti-human antibody cocktail for cell lineage sorting. hCD45, hCD3, hCD19, hCD13 antibodies were used. Dead cells were marked with 7AAD staining. Lymphoid and myeloid populations were sorted with the BD FACSAria II high-speed cell sorter. After sorting, cells were pelleted and stored at −80 C to proceed with DNA extraction and ddPCR analysis for HDR quantification as described above.

### Single-Cell Data

Single-cell data set generation: Single cell RNA-Seq was provided by the Single Cell Core at Harvard Medical School, Boston, MA using the 10× Genomics technology. Briefly, MLC sorted from competitively transplanted mice were isolated, and single-cell suspensions were prepared for each sample. Cells were then encapsulated in droplets containing unique barcodes and reverse transcription reagents, followed by library preparation and sequencing.

Data processing and quality control: The raw sequencing data was processed using the Cell Ranger software (version 4.0.0) [45] to obtain gene expression matrices for each sample. The resulting matrices were then imported into the Seurat package (version 4.0.4) [46] for quality control and downstream analysis. Cells with a low number of detected genes (<350) and high mitochondrial gene content (>15%) were filtered out. Cells with a total number of reads less than 3500 and more than 35000 were also removed from the dataset.

Normalization and scaling: The expression data was normalized and scaled using the SCTransform [47] function in Seurat. This method applies a regularized negative binomial regression to model the count data and correct for technical noise and batch effects. In the normalization process we included as covariates quantitative indexes reflecting cell cycle status (S.Score, G2M.Score), and percentage of cumulative mitochondrial gene expression. Gene Cx3cr1 was removed from the set of genes to avoid biases related to haploinsufficiency. The resulting scaled data was used for all downstream analyses.

Principal component analysis: Principal component analysis (PCA) was performed on the scaled data using the RunPCA function in Seurat. The top 12 principal components were selected based on their contribution to the total variance in the data. The resulting principal components were used as input for UMAP dimensionality reduction.

Uniform Manifold Approximation and Projection (UMAP): UMAP (McInnes et al. preprint, [48] was performed on the PCA-reduced data using the RunUMAP function in Seurat. This method projects high-dimensional data into a low-dimensional space while preserving the global structure of the data. The resulting UMAP plot was used for visualization and cell type identification.

Clusters identification: Based on the distance matrix calculated using PCA scores, the k-nearest neighbors graph (k=20, Seurat default) has been calculated. Clusters were identified using the Louvain algorithm. Different resolution values (0.6; 1.2) were tested and finally set at 0.8 based on the robustness of the clustering generated.

Cell type identification: Cell clusters marker genes were identified with the FindAllMarkers function in Seurat, which performs differential expression analysis between each cell cluster and the remaining cells in the data set. SCT normalized data were tested using the t-test option and setting the minimum fraction (min.pct) to 0.1.

TABLE 4
Gene editing reagents

| | Sequence | Targeting region |
|---|---|---|
| | gRNA for hCX3CR1 locus | |
| sgRNA 9 | TGATTCAGGGAACTGATCCA | EXON 5 |
| | (SEQ ID NO: 23) | |
| sgRNA PG5 | ACTATAGGGCTGGTAATCGT | INTRON 4 |
| | (SEQ ID NO: 24) | |
| | gRNA for Safe Harbor locus | |
| sgRNA S1 | GTCACCAATCCTGTCCCTAG | Integration site |
| | (SEQ ID NO: 25) | 1 (S1) region |

TABLE 5
TIDE PCR primers

| | | Amplicon | |
|---|---|---|---|
| Name | PRIMER | size | Sequence |
| sgRNA 9 | Primer FW | 700 bp | GCTGAGGCGGAAGGATAATC |
| TIDE PCR | | | (SEQ ID NO: 26) |
| sgRNA 9 | Primer RV | | GCCAGGTTCAGGAGGTAAAT |
| TIDE PCR | | | (SEQ ID NO: 27) |
| sgRNA PG5 | Primer FW | 812 bp | GCTGGGAAGCGAGAAGTGAC |
| TIDE PCR | | | (SEQ ID NO: 28) |
| sgRNA PG5 | Primer RV | | GCCTGGAGAGAGAAGCCTCA |
| TIDE PCR | | | (SEQ ID NO: 29) |
| sgRNA S1 | Primer FW | 650 bp | GATCCTCTCTGGCTCCATCGTA |
| TIDE PCR | | | (SEQ ID NO: 30) |
| sgRNA S1 | Primer RV | | TCTTAGGATGGCCTTCTCCGAC |
| TIDE PCR | | | (SEQ ID NO: 31) |

TABLE 6
Thermal protocol for sgRNA9

| | | |
|---|---|---|
| ANNY > TIDE CX PCR | Temp | Time |

| | | | |
|---|---|---|---|
| Activation | | 98° C. | 1′00″ |
| Amplification | 34 cycles | | |
| | Denaturate | 98° C. | 0′15″ |
| | Anneal | 65° C. | 0′15″ |
| | Extend | 72° C. | 1′ |
| Termination | | 72° C. | 5′00″ |
| Storage | 4 | 4° C. | ∞ |

TABLE 7
Thermal protocol for sgRNA 5 and sgRNA S1

| | | |
|---|---|---|
| Javi > TIDE CX PCR | Temp | Time |

| | | | |
|---|---|---|---|
| Activation | | 95° C. | 10′00″ |
| Pre-Amplification | 4 cycles | | |
| | Denaturate | 95° C. | 0′10″ |
| | Anneal | 55° C. | 0′20″ |
| | Extend | 72° C. | 1′30′ |
| Amplification | 34 cycles | | |
| | Denaturate | 95° C. | 0″20″ |
| | Anneal | 60° C. | 0′20″ |
| | Extend | 72° C. | 1′20″ |
| Terminate | 72° C. | | 5′00″ |
| Store | 4 | 4° C. | ∞ |

TABLE 8
HDR PCR primers (Table discloses SEQ ID NOS 32-43,
respectively, in order of appearance)

| Name | type | Sequence | targeted seq | Ruorofore |
|---|---|---|---|---|
| HDR | Primer FW | CTAGTTGCCAGCCATETGTTGT | on BGH | |
| CX | Primer RV | GAGAAAGEGAGAACCATGAGCAG | outside RHA | |
| PG5 | Probe FAM | CCCTGGAAGGTGCCACTCCCAC | on BGH | FAM |
| HDR | Primer FW | CTAGTTGCCAGOCATCTGTTGT | on BGH | |
| CX | Primer RV | ATTTGTTTCCACATTGCGGAGC | outside RHA | |
| sg9 | Probe FAM | CCCTGGAAGGTGCCACTCCCAC | on BGH | FAM |
| HDR | Primer FW | GATTGGGAAGACAATAGCAG | | |
| AAVS1 | Primer RV | TCTTGGGAAGTGTAAGGAAG | | |
| | Probe FAM | CCAGATAAGGAATCTGCCTA | | FAM |
| HALB | Primer FW | GCTGTCATCTCTTGTGGGCTGT | Albumin | |
| | Primer RV | ACTCATGGGAGCTGCTGGTTC | Albumin | |
| | Probe FAM | CCTGTCATGCCCACACAAATCTCTCC | Albumin | HEX |

### HDR PCR Thermal Protocol

1. 10′ 95 C
2. 20″ 95.5 C
3. 1′ 60 C
4. 1′ 72 C
5. Repeat 2-4×45 cycles
6. 10′ 98 C
10 C infinite hold

TABLE 9
List of flow cytometry antibodies

| Ab | Cat# | Fluorofore | Brand | Dilution |
|---|---|---|---|---|
| hCD45 | 368540 | PB | BioLegend | 2:100 |
| mCD45 | 103106 | PE | BioLegend | 1.5:100 |
| hCD19 | 302218 | APCcy7 | BioLegend | 2.5:100 |
| hCD3 | 317340 | A700 | Biolegend | 5:100 |
| hCD13 | 301712 | PEcy7 | BioLegend | 2:100 |
| hCX3CR1 | 17-6099-42 | APC | eBioscience | 2.5:100 |
| mCD45.2 | 5015801 | PerCP- | Thermo Fisher | 1:100 |
| | | Cyanine5.5 | Scientific | |
| hCX3CR1 | 2-6099-42 | PE | eBioscience | 2.5:100 |
| mCD45.2 | 109820 | Pacific Blue | Biolegend | 1:100 |
| mCD45.1 | 110738 | Brilliant | Biolegend | 1:100 |
| | | Violet 605 | | |
| mCD45.1 | BDB553776 | PE | BD | 1:100 |
| cd11b | 5016865 | AF700 | Thermo Fisher | 1:100 |
| | | | Scientific | |
| cd11b | 56-0112-82 | APC | eBioscience | 1:100 |
| mCD3 | BDB561799 | PE | BD | 1:100 |
| mCD45R B220 | BDB552772 | PEcy7 | BD | 1:100 |
| mCX3CR1 | 149008 | APC | BioLegend | 1:100 |

For the exclusion of dead cells, either 7-AAD (1 mg/ml) or DAPI (Sigma-Aldrich) were used. Cells were analyzed at LSR Fortessa instrument or sorted at BD FACSAria™ III Cell Sorter (Becton Dickinson, Franklin Lakes, NJ, USA) at the Flow Cytometry Sorting Facility of Dana-Farber Cancer Institute (Boston, MA).

### REFERENCES

1. Biffi, A. et al. Lentiviral hematopoietic stem cell gene therapy benefits metachromatic leukodystrophy. Science (1979) 341, (2013).
2. Sessa, M. et al. Lentiviral haemopoietic stem-cell gene therapy in early-onset metachromatic leukodystrophy: an ad-hoc analysis of a non-randomised, open-label, phase 1/2 trial. The Lancet 388, 476-487 (2016).
3. Eichler, F. et al. Hematopoietic Stem-Cell Gene Therapy for Cerebral Adrenoleukodystrophy. New England Journal of Medicine 377, 1630-1638 (2017).
4. Gentner, B. et al. Hematopoietic Stem- and Progenitor-Cell Gene Therapy for Hurler Syndrome. New England Journal of Medicine 385, 1929-1940 (2021).
5. Fumagalli, F. et al. Lentiviral haematopoietic stem-cell gene therapy for early-onset metachromatic leukodystrophy: long-term results from a non-randomised, open-label, phase 1/2 trial and expanded access. The Lancet 399, 372-383 (2022).
6. Cartier, N. et al. Hematopoietic stem cell gene therapy with a lentiviral vector in X-linked adrenoleukodystrophy. Science 326, 818-23 (2009).
7. Cartier, N. et al. Lentiviral hematopoietic cell gene therapy for X-linked adrenoleukodystrophy. Methods Enzymol 507, 187-98 (2012).
8. Chetty, K. & Booth, C. Gene therapy for primary immunodeficiencies: up-to-date. Expert Opin Biol Ther 21, 529-538 (2021).
9. Goyal, S. et al. Acute Myeloid Leukemia Case after Gene Therapy for Sickle Cell Disease. New England Journal of Medicine 386, 138-147 (2022).
10. Hsieh, M. M. et al. Myelodysplastic syndrome unrelated to lentiviral vector in a patient treated with gene therapy for sickle cell disease. Blood Adv 4, 2058-2063 (2020).
11. Tucci, F., Galimberti, S., Naldini, L., Valsecchi, M. G. & Aiuti, A. A systematic review and meta-analysis of gene therapy with hematopoietic stem and progenitor cells for monogenic disorders. Nat Commun 13, (2022).
12. Gentner, B. et al. Identification of hematopoietic stem cell-specific miRNAs enables gene therapy of globoid cell leukodystrophy. Sci Transl Med 2, 58ra84 (2010).
13. Bosch, M. E. & Kielian, T. Neuroinflammatory paradigms in lysosomal storage diseases. Front Neurosci 9, 1-11 (2015).
14. Subhramanyam, C. S., Wang, C., Hu, Q. & Dheen, S. T. Microglia-mediated neuroinflammation in neurodegenerative diseases. Semin Cell Dev Biol 94, 112-120 (2019).
15. Pawelec, P., Ziemka-nalecz, M., Sypecka, J. & Zalewska, T. cells Neurological Disorders. 1-17 (2020).
16. Pawelec, P., Ziemka-Nalecz, M., Sypecka, J. & Zalewska, T. cells The Impact of the CX3CL1/CX3CR1 Axis in Neurological Disorders. doi:10.3390/cells9102277.
17. Wolf, Y., Yona, S., Kim, K. W. & Jung, S. Microglia, seen from the CX3CR1 angle. Front Cell Neurosci (2013) doi:10.3389/fncel.2013.00026.
18. Jung S et al. Analysis of fractalkine receptor CX(3)CR1 function by targeted deletion and green fluorescent protein reporter gene insertion. Mol Cell Biol. PMID: 10805752, (2000).
19. Capotondo, A. et al. Brain conditioning is instrumental for successful microglia reconstitution following hematopoietic stem cell transplantation. Proceedings of the National Academy of Sciences 109, 15018-15023 (2012).
20. Peviani, M. et al. An innovative hematopoietic stem cell gene therapy approach benefits CLN1 disease in the mouse model. EMBO Mol Med (2023) doi:10.15252/emmm.202215968.
21. Capotondo, A. et al. Intracerebroventricular delivery of hematopoietic progenitors results in rapid and robust engraftment of microglia-like cells. Sci Adv 3, (2017).
22. Verdonk, F. et al. Phenotypic clustering: a novel method for microglial morphology analysis. J Neuroinflammation 13, 153 (2016).
23. Garin, A. et al. Two Novel Fully Functional Isoforms of CX3CR1 Are Potent HIV Coreceptors. The Journal of Immunology 171, 5305-5312 (2003).
24. Garin, A., Pellet, P., Deterre, P., Debre, P. & Re, C. C. Cloning and functional characterization of the human fractalkine receptor promoter regions. Biochem. J vol. 368 (2002).
25. Schiroli, G. et al. Precise Gene Editing Preserves Hematopoietic Stem Cell Function following Transient p53-Mediated DNA Damage Response. Cell Stem Cell 24, 551-565.e8 (2019).
26. Lombardo, A. et al. Site-specific integration and tailoring of cassette design for sustainable gene transfer. Nat Methods 8, 861-869 (2011).
27. Konturek-Ciesla, A. & Bryder, D. Stem Cells, Hematopoiesis and Lineage Tracing: Transplantation-Centric Views and Beyond. Frontiers in Cell and Developmental Biology vol. 10 Preprint at https://doi.org/10.3389/fcell.2022.903528 (2022).
28. Capotondo, A. et al. Intracerebroventricular delivery of hematopoietic progenitors results in rapid and robust engraftment of microglia-like cells. Sci Adv 3, (2017).
29. Butovsky, O. et al. Identification of a unique TGF-β-dependent molecular and functional signature in microglia. Nat Neurosci 17, 131-43 (2014).
30. Mizutani, M. et al. The Fractalkine Receptor but Not CCR2 Is Present on Microglia from Embryonic Development throughout Adulthood. The Journal of Immunology 188, 29-36 (2012).
31. Kierdorf, K. et al. Microglia emerge from erythromyeloid precursors via Pu.1- and Irf8-dependent pathways. Nat Neurosci 16, 273-80 (2013).
32. Mizutani, M. et al. The fractalkine receptor but not CCR2 is present on microglia from embryonic development throughout adulthood. J Immunol 188, 29-36 (2012).
33. Park, H. & Cox, D. Syk Regulates Multiple Signaling Pathways Leading to CX3CL1 Chemotaxis in Macrophages * □. 286, 14762-14769 (2011).
34. Wang, L., Yang, L., Filippi, M., Williams, D. A. & Zheng, Y. Genetic deletion of Cdc42GAP reveals a role of Cdc42 in erythropoiesis and hematopoietic stem/progenitor cell survival, adhesion, and engraftment. 107, 98-105 (2006).
35. Williams, D. A., Zheng, Y. & Cancelas, J. A. Rho GTPases and Regulation of Hematopoietic Stem Cell Localization. 439, 365-393 (2008).
36. Yang, L. et al. Cdc42 critically regulates the balance between myelopoiesis and erythropoiesis. Blood 110, 3853-3861 (2007).
37. Subbarayan, M. S., Joly-Amado, A., Bickford, P. C. & Nash, K. R. CX3CL1/CX3CR1 signaling targets for the treatment of neurodegenerative diseases. Pharmacol Ther 231, (2022).
38. Cromer, M. K. et al. Ultra-deep sequencing validates safety of CRISPR/Cas9 genome editing in human hematopoietic stem and progenitor cells. doi:10.1038/s41467-022-32233-z.
39. Visigalli, I. et al. The galactocerebrosidase enzyme contributes to the maintenance of a functional hematopoietic stem cell niche. Blood 116, 1857-1866 (2010).
40. Denning, S. M., Tuck, D. T., Singer, K. H. & Haynes, B. F. Human thymic epithelial cells function as accessory cells for autologous mature thymocyte activation. The Journal of Immunology 138, 680-686 (1987).
41. Bak, R. O., Dever, D. P. & Porteus, M. H. CRISPR/Cas9 genome editing in human hematopoietic stem cells. Nat Protoc 13, 358-376 (2018).
42. Hsu, P. D. et al. DNA targeting specificity of RNA-guided Cas9 nucleases. Nat Biotechnol 31, 827-832 (2013).
43. Brinkman, E. K., Chen, T., Amendola, M. & Van Steensel, B. Easy quantitative assessment of genome editing by sequence trace decomposition. Nucleic Acids Res 42, (2014).
44. Ferrari, S. et al. Efficient gene editing of human long-term hematopoietic stem cells validated by clonal tracking. Nat Biotechnol 38, 1298-1308 (2020).
45. Zheng, G. X. Y. et al. Massively parallel digital transcriptional profiling of single cells. Nat Commun 8, (2017).
46. Butler, A., Hoffman, P., Smibert, P., Papalexi, E. & Satija, R. Integrating single-cell transcriptomic data across different conditions, technologies, and species. Nat Biotechnol 36, 411-420 (2018).
47. Hafemeister, C. & Satija, R. Normalization and variance stabilization of single-cell RNA-seq data using regularized negative binomial regression. Genome Biol 20, (2019).
48. Becht, E. et al. Dimensionality reduction for visualizing single-cell data using UMAP. Nat Biotechnol 37, 38-47 (2019).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A promoterless splice trapping cassette polynucleotide comprising from 5′ to 3′ a first homology arm derived from an intron or exon of a target gene, a splice acceptor site (SA), a polynucleotide encoding a heterologous polypeptide, and a second homology arm derived from an intron or exon of said target gene.

2. The promoterless splice trapping cassette of claim 1, wherein:
the target gene encodes a CX3CR1 polypeptide;
the intron is intron 4 of a CX3CR1 polynucleotide;
the exon is exon 5 of a CX3CR1 polynucleotide; and/or
wherein the heterologous polynucleotide encodes a therapeutic polypeptide.

3. The promoterless splice trapping cassette of claim 2, wherein the therapeutic polypeptide is selected from the group consisting of: a lysosomal polypeptide associated with lysosomal disorders; a polypeptide associated with peroxisomal diseases; a microglia-associated polypeptide and/or receptor; a neuromodulating polypeptide; and a polypeptide associated with the pathogenesis of neurodegenerative disorders.

4. The promoterless splice trapping cassette of claim 1, wherein the first homology arm and the second homology arm each comprise at least about 250-1000 base pairs of a target gene intron or exon.

5. A vector comprising the promoterless splice trapping cassette of claim 1.

6. A vector comprising from 5′ to 3′ a first homology arm derived from a CX3CR1 target gene, a spleen focus forming virus promoter, a polynucleotide encoding a heterologous polypeptide, and a second homology arm derived from the target gene.

7. A system for editing the genome of a cell, the system comprising a polynucleotide comprising a promoterless splice trapping cassette polynucleotide comprising from 5′ to 3′ a first homology arm derived from an intron or exon of a target gene, a splice acceptor site (SA), a polynucleotide encoding a heterologous polypeptide, and a second homology arm derived from an intron or exon of said target gene, a Cas polypeptide or a polynucleotide encoding said Cas polypeptide, and a sgRNA that directs binding of the Cas to the target gene.

8. The promoterless splice trapping cassette of claim 7, wherein:
the target gene encodes a CX3CR1 polypeptide;
the intron is intron 4 of a CX3CR1 polynucleotide;
the exon is exon 5 of a CX3CR1 polynucleotide; and/or
wherein the heterologous polynucleotide encodes a therapeutic polypeptide.

9. The system of claim 8, wherein the therapeutic polypeptide is selected from the group consisting of: a lysosomal polypeptide associated with lysosomal disorders; a polypeptide associated with peroxisomal diseases; a microglia-associated polypeptide and/or receptor; a neuromodulating polypeptide; and a polypeptide associated with the pathogenesis of neurodegenerative disorders.

10. A cell comprising the promoterless splice trapping cassette of claim 1.

11. The cell of claim 10, wherein the cell is a hematopoietic stem cell or progenitor thereof.

12. A method for inserting a heterologous polynucleotide in the genome of a cell, the method comprising contacting the cell with the system of claim 8, thereby inserting the heterologous polynucleotide into the genome of the cell.

13. A method for enhancing engraftment of a hematopoietic stem cell or progenitor thereof expressing a therapeutic polypeptide in a subject, the method comprising
a) contacting the hematopoietic stem cell or progenitor thereof with a system comprising:
(i) a promoterless splice trapping cassette polynucleotide comprising from 5′ to 3′ a first homology arm derived from an intron or exon of a CX3CR1 target gene, a splice acceptor site (SA), a polynucleotide encoding a heterologous polypeptide, and a second homology arm derived from an intron or exon of said CX3CR1 target gene;
(ii) a Cas polypeptide or a polynucleotide encoding said Cas polypeptide, and
(iii) a sgRNA that directs binding of the Cas to the CX3CR1 target gene, wherein contact with the system inserts the heterologous polypeptide into the CX3CR1 target gene, thereby generating a CX3CR1 haploinsufficient hematopoietic stem cell expressing a heterologous polypeptide;
b) administering said hematopoietic stem cell or progenitor thereof of a) to a subject that has undergone myeloablation.

14. The method of claim 13, wherein the intron is intron 4 of a CX3CR1 polynucleotide.

15. The method of claim 13, wherein the exon is exon 5 of a CX3CR1 polynucleotide.

16. The method of claim 13, wherein the subject is a human.

17. The method of claim 13, wherein the method reduces or eliminates expression of the CX3CR1 gene in the cell.

18. The method of claim 13, wherein the method enhances engraftment of the edited hematopoietic stem cell or progenitor thereof in bone marrow or brain of the subject relative to control hematopoietic stem cell that is not CX3CR1 haploinsufficient.

19. A method of treating a neurometabolic or a neurologic, or neurodegenerative disease in a subject in need thereof, the method comprising
a) contacting the hematopoietic stem cell or progenitor thereof with a system comprising:
(i) a promoterless splice trapping cassette polynucleotide comprising from 5′ to 3′ a first homology arm derived from an intron or exon of a CX3CR1 target gene, a splice acceptor site (SA), a polynucleotide encoding a heterologous polypeptide, and a second homology arm derived from an intron or exon of said CX3CR1 target gene;
(ii) a Cas polypeptide or a polynucleotide encoding said Cas polypeptide, and
(iii) a sgRNA that directs binding of the Cas to the CX3CR1 target gene, wherein contact with the system inserts the heterologous polypeptide into the CX3CR1 target gene, thereby generating a CX3CR1 haploinsufficient hematopoietic stem cell expressing a heterologous polypeptide;
b) administering said hematopoietic stem cell or progenitor thereof of a) to a subject that has undergone myeloablation.

20. A sgRNA comprising the following sequences UGAUUCAGGGAACUGAUCCA (SEQ ID NO: 1),ACUAUAGGGCUGGUAAUCGU (SEQ ID NO: 2), or GUCACCAAUCCUGUCCCUAG (SEQ ID NO:3).


SEQUENCE LISTING (43 sequences)

SEQ ID NO: 1 - RNA, 20 nt, synthetic construct
  source 1..20: mol_type = other RNA
tgattcagggaactgatcca

SEQ ID NO: 2 - RNA, 20 nt, synthetic construct
  source 1..20: mol_type = other RNA
actatagggctggtaatcgt

SEQ ID NO: 3 - RNA, 20 nt, synthetic construct
  source 1..20: mol_type = other RNA
gtcaccaatcctgtccctag

SEQ ID NO: 4 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 5 - DNA, 1487 bp, Homo sapiens
  source 1..1487: mol_type = genomic DNA
ggggcagatccagattccctttgcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtccttcaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaaagggtgtctgagccctcaaagtgaggggaaccagggcctgagccaagcta

SEQ ID NO: 6 - DNA, 83 bp, Homo sapiens
  source 1..83: mol_type = genomic DNA
gaaatactcgtctctggtaaagtctgagcaggacagggtggctgactggcagatccagaggttcccttggcagtccacgccag

SEQ ID NO: 7 - DNA, 3023 bp, Homo sapiens
  source 1..3023: mol_type = genomic DNA
gccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttc

SEQ ID NO: 8 - DNA, 13435 bp, Homo sapiens
  source 1..13435: mol_type = genomic DNA
gtaggtgcacaggactagttgggtacctgtgggtggggtggagcagtggacagctaataggttaataatgcctgtttgcttacgtgcagacaatggaaaccattttcctggggatgttgtagcctaaatatgtccaaggggatggaagagtgggaggcaaggggtgatcagatcatttataatacactcaacctggtggaatagtattagaagcattagtaattacattttagagacatggagaaaagctcatgattttaaactaactgaaaaaagcatgaaaaattgcatctggatgctgttgaggaattattgctaattttttgagatgagaaattgtattaaagtctttttcaaaaaagagtccttaacttttagagatgcacacaagtgtttatgggtgaaattaaataattcagtagagacattaagtgggaaatagaggaaatattgaccatgggttgctaatagttgaagccaggtgttgggtataaggaggttctcgctgcttttatttgaaaagttgctttttatttgaaaatttaataataaagagtttttaaatttgtatctgtattttaatataaatataaatgcacttaatataaatataaaatatgtataacatttagatagagaaaagctaaaagattactgtggttgattctaggaaactgcattgcagatagtttcatgggtttttttttcctttttcttccactttttgtaccatctatgggtttttgtttgttttttgttttttgggttttctttcttgtttgtgttttgttttttgagacggagttttgctcttgttgcctaagctggagtgcaatggcacagtctcggctcactgcaacctctgcctcctgggttcaagtgattctcctgcctcagcctcccaagtagctgggattataggcatgtaccaccgcccggctaattttgtatttttagtagaggcggggtttctccattaataaattcctggcacaaatttagtgttcaattttgatatatgttgttataaccattgtgaggatactcaggctcaggtttgtgtgggtggaaaacatggtcttcagaaagaaattatgagtgcaagacaggaggaaatccatcagaggccccagctgaggactgaccacggcttgttatttctcttgccttgcctctggcaatcacagcctcacagagcctgcaatccttgctttgtgagtttatagctcagtccagagaatggctaagaaagtttaggattctttcaacacccactccacaaaaaaaaaaaaaaaaaagaaaagaaaaaaaaattaatttttgaaatacttgaggtagaaaacttgaggcagaaaaaaattgagccaaaaaaaaaggaaaattgaaccacgtgaaagcaggcaagaaagcttgcattgctcagggcatcccaggcccagagggcgctttggagggagctgggtttcctgagaggaggcagggtgggtgacggacctgtgctggagagccttgaggaccactgtgggttgggaatgggggcagtggattggggttcaaaacccctgggaatgagaaatgggctcaggaaggctagggtggattctttcatcttcctctttgcttggctttattttcacaaaggaaggcagggcaggaaatagtctcagcccaacttcagtgtggttcttcttagtgctcaggcttacctggcacttgccacacctctgggatgggagcacctactatccatcagccacgtgccagtctccacaaagtctgctcctgaaccctgctcctcagctggccccacttcacagatggggacataggcagcttggctttggaatgaaggaatgaagtcaggaatgaagtcctggctctgcacttggtgactgtgcactgggcttgctaagtctgtttcctgcttttaaaatggagattgtccatcagcctttgaagccatgtaatgggtatgtgtcaactttctgcaaggattaaaggcatggtataggaagtcccaaacacactgcctgacccatctttgatgctcaagaaacgatatatgttgttgtcatgaggaaactgagcctcagaaagtttggatacctgaaaaacactgactactattgaatgaggttgtgaagaatccagagctgtaggggcaggaaagcaaagaacgtattagagctgacccagtcaggacgatcgtctatccccttcctcaccccaccccatcccaggaggaagcctgcccggccctaggcagctatggcacagtggcaatgtcaggtatggttctccctagccagagaccctagcctcaaaaaacctccttcttgggatccaggcatccaactgctcctccccagccccagcctctgacccagtatcctgagtccagagacgtttggaaccagcacctgtaatggaggagctgaacaaggaggggaacttctgctgctccacagcaggtcacggtcataggagggagtggaaccagaatggcagaatccagatcttggctgcctttcccaaggacttgttctgattcctagcagcacagcccaggcattccgagaagttgggctctctggcatcactcactctgcccagaagagccaggggaaagttggggcttctagctgaaccttgatcccacctgccctcttgaggggctcagaatctgctggctgcttcacaggtgggattctcacggcacgctggccacagctgatgcttcgaccccctcatcttgtttggccaaagtgcagctttttagcttgtgagtaaggaagaaaagctgtatcatatgtctttaaacatcttcctagaccacctttgttttccccttaaagtgtgcttaaggagaaaatggaaagtctcttttcagtgtgttgtattttgtttattgcaaaatacaacacactgaaagaacaatgtctgggttagcaaagtattagatttaatttcccacattactaccatccaggctgagaaagagaaccccagaagcctctctcatgtcccttcctgaccacagcctcttcctctcccacaaagaaccacaagtctgacttctatggtgatcacttccttgcttttccctatagtttgactgaatctaaatatcatccctaaacaatatagcttagctttgcctatttttgatcttcatatttggattcatactttttggtttggctcaagactaaacttttaaaatgcatccacgttgtaggagatgtacatgcacaaaaattttcacggcaacattgtccaaactagcaacaaactgaaagccacctatgtgtgcattagcagtagaaatggcaataaatcatggactagtcacacagtggaatcctacgcagcaaggagaattagcagtctacagccaaaccaacagcatgggtgagatgcttccagaaatactgagatgaatttagagataaaggcacaattggtccatatctacagggcacttaatccagtgcatctccactagacaaaatttgtatttccatgagcaagagtcatttgcactgctatctgctataacctacggtgctgtaaaattagctcaggcaataaaagggggaggtagccccaaagagtatgccagaaaggactcccagtaatcttcagtgtgtgtctatttcaaagtctttcatcatttttccttgtagttacttagtgtggaatttcagacccctctctatgtccctcttctcccttttcagccttggctttctctgcatccttccccgaagccctgcactcagcctaaactgactttcctgaacactctgggagcatgtgggctcctcccaactccaccatcttcactgccttctgttcctatttccctctgcctggccttggccccaggaaaccttcctatgctcagaccctctgtgcctttgtcttcaaagcccacccactattcactgccacctccatcttggtcaacctggaagactttccccatccttaaaacatcagctcaaacggcagatttttttttttttttttgtgatatcctcttgatcaccctcccccagtttttaaggagacagatatagaggttcaatccttgggcttcccatgacttttcttatattctgttactgagcaataataaacacttctaggaactgtatcttaaacacttttgcctctacagaatctagcacagtgcctagtattggtgaaatataattaacaaagtcttctttcaacacagagattctctccacaaaaggagtagagaaagaacagttttattatggaataagcagtaaaccaaaatatgcagagcattataggccatctactaagaggttgcaagaacagaaagaaatctcccccttttgtatagccaagtagatacaacctgttacatacatgttatcaaggcaaacaataactgttcctcaagtaagaggtcttgccagcaccgtttgccgtacatggttcactctaaatttacctggtaattagggtaaccacttgtgttagctaactggctctacccagaggaaaatcaaatttatctttaagacaaggggtaattttgcagcactgagcaaggctcttcagttaggctcataccttcccacagaaactaagagatagaagcactatctcccttaggtttgtttacatttcaaagagatgacgcccaggtccttgggaaagactagcttagctcataaagctgacaaaaagcctatctagtttcaaaaggatttacacatgttcaaagagaggagaaagtatgtaaaagttttctaggtgggcacggtggctcacacctgtattcccagcactttgagaggctgaggcgggtggatcacctgaagtcaggaatttgagatcagcctggccaacatggtgaaaccttgtctctactaaaaatacaaaaattagctgggcatggtggtgggcgcctgtaatcccagctacttgggaggctgaggcaggagaatcacttgaacccaggaggtggaggtggcagtgagccaagatggcaccattgcactccagcctgggcgatgagagtgaaactccatgtcaaaaaaaaaaaaagttttctaaagtaaatgctctgagaaaaaaacagaaggtgaggagatctctgtccttatttttaacaagaataattaaatgtttttatttttaatttctacttagactagaatatggtacagatgctcaaaaatattgacttaattaataattaaacaattaacaagtctatctctttcctaattattaaaaaaattatgaagtatttcccttcaaaatttcacagtttactttgacataaataaataaataccataaagtattttgaagaggaaagggaagggtggggcttcaaacacaaaataatggcaggttttgtaaaggggcaattcactaaacccaataggattctcctgttgctattttttttttttttttttttttggtggtggttggagggaagggttgggtgtgcatttggcctgtgtgaaaaactggtgggtagacaatgccatgctgttctagatgggctcattgctataggaatagatagtcactgaatttacttgcataggggatggggtataaaatgtttccttatagctccttaacatggaattcagcacccctctccccatcagccttagctctctctgaatcccaagggatatgtgttataaaaatagctgccatcataaatgagaaaaccatcaggacctggtaaaacttggcttcccaaacagtcaggggtctgggcatggcagccgactgagaacctttctatctagtacaaggtaaacaagatttgccaagacttgatcaactgatccagctccctatctccaaacagaacctcatctgaatgctcacaggctgtgccccagaagtgtagaagtgtcatgttccctgggcaggctgctgggcagcctctctgttgacaatagaccacacttttgctgacctaggactcatgttgctctttaagactgcttccttggccaggcatgatggctcacacctgtaatcctggcactttgagaggcccaggcaggaggatctcttgaggccagatgttcaagaccagcctggtcaacatagtaagaccccatatctaccaaaaatagctgggcatggtggtgcacacctatactcccagctacttaggagactgaggtgggaggattgctacatcccaggagttcaaggctgtagtgagctatgatcatgccactgcactccagcctgggcaacagagcgagatcctgtctcaaacaaacagtttccttctgtttgattcttgctgaaaaattgagcatgccagagctagcaaggctcttagaggtggtctggtccaatgctttaatttcatacatcaagaaactgaagcagagcagagtgacctgcccagggtctctcagccattcatgctcagaaatgtatgggctcctgtgaaacatgtggctcttaaaagcactatcatatatttgaaggcagaaaataggctaaaccttcagccttcagacttttcctctccagagaaaatgaccccagtttcctcactatggttgctgggagctagattcctggggatctggcagtgtggaccacctagtggtggctagaggagcaaataatatcccgcattccattttccactcaccaatccctgaggggcagcctgctgggttatgagcccacagggggagaaccccaacgaattcagagatgcatcatggaccagtttctcttaaggggcctgggtctactattttcagttctacttcgagagaagtggcctgcaatatcctgcagatttcccctccagggagaaaagcattgtgcggtgcaaggagcacaggctttgtggaaagaggcatctggggttgagatcctggctcttttgcttccttgaacaagttaaccaaatctctgggcctctgctggttaatttataccatggggatcatcatttctccagtggggttgtgaagagaatgtggtgggatcttgtgtgtggagcatgacacttagcaggcatccgggaatggcagcctcctccctttctaaactggggctttctgagggtgacttcagattccacaatgtcaacagcacaatggcatcctcataaggaaagtttgggttggggctcctcaagcaattctctactctcatttggtacaaagaaaaaaattaagcctcacaattttcttggcaccagactgaacctcaaacccagtcttcacttttactaaaaagccataaacagagaccaggagggtaaaaactaccagaagatacactggatttagaaaacagtaagctggatgtgaagccaaaagagagggagaaagcaacataaaagaatgtcagattgaagaagaaatggattctggccacctaaagaagaagggaagtatagaaaagggaagctgattggaaacaatgggtaatgatgagtgtccctcccctaaaaagttaaaaaataaatgagcaggcatgagaaaaggaagtaggccagaggaagtagaccaggatcagccacggatgtgagtggggaattttacaaatttctacttgggtgtgaaacatgtgtacactcaaagctctgtctaggttcaccgaaactctcccagctcaaggccatttagtaaaaccttggcttaactgaatagtgggtaaggaggcctgaggatggcccagagaaggtttaaattcttactggctgctgcactaagttgtaaagtgcagccttagttgatgatgcctttattagtgcaacatcccagaatgcatgcgtcttacaacccttgtggatatgaaacccagctgggtagacactgcaaagtcttctcatttgattcttccacttggtttctagtgtccctcagggacaaaggaaagccatggtccagtctaagatgaaaaccaattagacctctggcaggcctttgaccaccctgagggccacccccaaggcacggccacactcgcatctgctgccaggaggccctatcttaggctcagctcccaaaccttgaacatcttggaggatccacaaataggaggtacactcagctcaggctcagtcttccttaaaaagcgccttgctaaagctatcaagttcactggaatacttctgcgaaggacacagcttcagcgatgtcagattttttatgtaaatggtgccttcacatgctctggggcatttggctaccaagggcggtttgaactagctccagcacacagaatacaagtctcttcagaaccaggcaaaaccctatgttgcccaatgactcctgctgtttcttgagatttgcacagaagacagagctgcaattacccacgctgatatctatttcatgaccacatatgttcaaaagccacatgtgagaggtatggttgaaatgagaggttgtgtttgccaagttgtcttctgactgtggagagctggtgagcctcttctcatctcctggggctccatattatagagctgacgaatctcttttcttgctccttgaagtctttcattcacttatttattaattcatttaacacatcaggcacctactaacttctcagagttcaaggcttcctagcctatgatcagaaaggacacctgtgtgctcatgagcacccctgggatcagggtgactgatagggtgctgtcatcactactagatgaaactctttggaacaaaggtctaggatataatatttatccttctgcaaatattcatatggcattcgctgtgtaccaggccttctgttgaacattggcctgcagagctgaagaggacgtgggtcttctcttaggaactcctagtcttgggaaaaaggaatggggaagggctgtaatgtgaagttaaaaaaaagtgctgaggccgaacacatctgggccctcactgcagccctattgcacacacactgcatcacctcgaacaagttattcaccctctctgaattcatctatttgcccataaaaatagagatgatccttttatatgttggctaccacttattatgtgcagcatgcttgttattgtactaagttactcattttttagtttttcatataactcatatttttgttgatttttattttaaaattccaaacgaattaattaaactattttccaaaatacgtagtatgtgtacatggtaaaaaacatttccaaaagtttaccataaaaactgtctcctttcccatatcctcaatccttcagaatccctctccagggataattaccaccaccagtgtattccttaagagatatttaaactttatacaaaatacgcacacatcattcttttccacaaatgacagcatactatgtacatggtacctcacctagcttttttcacttaccagtatatcttagagattgttgcatgacagaatatacagatctgcctgtttttgttgtttttcccaagtttccaagagctagaaactgtttggtttttaagcagctgcttggtattccattaattggacttatcgtgctttgatctgtccctagtgatggacattggggttgtttcccttcatttatatttgaagcttgttgcagtgacagtgtacacacttgcttgagcatgtgtgtgtctacgataaatcccaataagcaatttaaagtaatttaattcttatgctctcaacaaacctaagaggttatttttttagaggaggaagctaaggctgttgttttgatgcctttttaccactggacttgggactcaactgtttgatagtcatcgagtccttgactcttctcccctgccaggatttggcccctaagcccagggcccgagtctcctccatcttcaagggagagtgggaacagcacaggccttggcctcagtcctgctccccctgcttctagctgtgggatgggccaggtgcttcacccagctgtgcctcctgtgaggcacagtgtgtgcaaaatggaaatgtgaacatgaagatcaaaaatgtccctcaatgaccagtgctgttcctcagagtcacgtgggaactcatagaaagcgatattggtactgctctttcctctgtagcatggtccagatggctcatagcagggaccatgatatgctgggtgagcacccactgcatgcacccactgtgccagcactgagagactcctgtgggagccacagcaattctagggtcttcactggggactctgagacagcagggagctaggatgagggctgcagagtgttcgtctgccctcactgagcagaccccctggatggcagggagcagtcccaagccagatggatgcccataaccagccatttggctctcaatacataatatcaccacgtatcaggcaaaaccatcctgcccagagcattatctgaatttgcatcccatctgcagaagatacattcacccacttcttccattctgtcttaatcaaagtctttatgtgaattttccccattgagaagacaagccccttcctggcttagactgtacctgactgatcttttcatgagctccttgccaagccagaccacccccagcttatatggagacttggtgcaaattagagatgcccctgtgcacgtggcagccctgagcccaagcacccagtaaggcaaagggcctgatttgggacccctctgccactccaccaggcaatcagttgcttatttctaactttcccttccttctccacatttgtcccattccttcctctcatcatgaatatccccagaggcattcagcagtgcagtgaattaaatatagaacttttttttttcagaattgcagaacggattagatcaatattaatccaaacagagcaatgagcctgacagtttagtaaaagctcaataaagggtggcttacctcccccaaaataatctgaaaagaaagcatgtcttatttcagggggaaaaaaaaataaagtgacctttaaagaccaaattcccaggatacccagggtggaggtggaacatgggagtccacaggcagcctggatgtttccaaagatccaaagggcttttgcttcctcacataatgcaggaaacaaattgaacatgtattaagtgcttgctgtatgtgacacactgtgccaggtgctccccttaaaacagttctgtgggcaggcatgagaatgaatcccgatcttacagacaaggaatgttaggctcagaggtttcaagctcacccatcactcagccagagaggacagatgcaggattcaatctcgggagtgcccgagtccacagaagttcctgtgctgaaggaccgaccacaggcacataaagagatgcgagacaatttttactggatttggccacctctcgaggtcggctttgccagctcttctcactgggggaaggggagggagaaagtagctagctccagggtccctaacatagaaccaccaaggacttgactatttttactcatacagcagcttgtctgggaagatcatgctctgtgacaagctgcaggcactaagtagcaatttctgtttcccacatattagcttgagtcatataaaactgacatggatgtggctcaaaaatagctgtatgtcagccattttataccatttgacttaaatgttattaattaacgtcacagccagagattattctctgagaaaagggcattgtagcctgaagcagagaaagcatacacgttccctggggttgagaactcatcacagcctgagacagcttaggttgtaaagccccggcccacttatcccaggagagtctgggtgagatgcaggccccaaagcagaggctgggaagcgagaagtgacacaccctggctgggtgggccctcatcttggtgagacaccacctgggtaaaaccatcatggaaagggtgtagtggggcgtggaaactccctcggttaaagcgtgagctttgctgtaagttgtggtaaggagggaggcagtgacaaccaggaggcctgttttgagggtttctgagggacccatctgtggtatcacgaggagacgcccagaggagccgtgtgaaagggctgcctcccagccggctctggagtgaatgagcagcaagtcctggctgcgaaaagaaggggagtgcagcctgcagaagtgtcttcttttttcaattcctgctcagaaggaaacaggagataagaatagtggggaagtccaaaccaaagtgaactatagggctggtaatcgtagggggaattagtcacccggagactagcccagcagactaacggagccccatcctccatcttgaatcagtcagcccctctatgactgcagagtcctgaatgatggcaacaccttctcttcacttagcgttgtaggatgaccaacagtcctgatttgcctgggactgaggggttcccaatagatgggactttcagggctaaaaccaggaaagtcctgggcagcccaaaacaaggtagtcactctagagtgtatgactctgtctgatacctgctaagaaagagaaggacttgttgattataaggagaagaggaggtgaaatggttctcaaaaaacaaagatgagggcttccgggtgctgttctgcccaaggctctgggtctgaggcttctctctccaggcctagcttcatggaaaagtaaggggccagagggtggaaaaggtggaaacaaaggaagaggatggagaattgctttggggaagtttggactggaagtgtgaattacagctgcacccccaattcaccccatctcacccccctccccctcctgctcatggttctccctttctcatccacacattggtcaaactagctagcttttggagagattttgggcagtaaaagtaaaacagatctgtctcaagcttcaaaaagcctagagctggctgggcgctgtggctcacgcctgtaatcctagcattttgggaggctgaggcggaaggataatctgaggtcaggagtttgagaccagcctggctaacatgatgaaaccccatctctactaaaaatacaaaaattagccaggcgtggtagtgcacgcctataatcccagctatttgggaggctgaggcaggagaatcgcttgaaccccaggggacagaggttgcagtgagctgagatcgcaccactgcactccagcctgggtgacacagcgagactccatttaaaaaaaaaaaaatgcctagagccaaatgctcacagagccatttactgcatggctttgggcaagtcaaaggagtccgcctctcctgtcagaagagtctgttgcagtcttcatcacaagactgttgtggggattaaacaagatggcaagtgggaagttgggaaatgtagtgtgcacccaaccaatatttgtttcttcctgcctgcctacatatgaggccacacagaattccaactttgtttctctgataactaacacagttacttgtttttctttctgatccag

SEQ ID NO: 9 - DNA, 1946 bp, Homo sapiens
  source 1..1946: mol_type = genomic DNA
agggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttc

SEQ ID NO: 10 - DNA, 92 bp, Homo sapiens
  source 1..92: mol_type = genomic DNA
gaaatactcgtctctggtaaagtctgagcaggacagggtggctgactggcagatccagaggttcccttggcagtccacgccaggccttcacc

SEQ ID NO: 11 - DNA, 16641 bp, Homo sapiens
  source 1..16641: mol_type = genomic DNA
atactattggctaactctgcaaataaaatgctcttagagggaaggaaagggaaatactcgtctctggtaaagtctgagcaggacagggtggctgactggcagatccagaggttcccttggcagtccacgccaggtaggtgcacaggactagttgggtacctgtgggtggggtggagcagtggacagctaataggttaataatgcctgtttgcttacgtgcagacaatggaaaccattttcctggggatgttgtagcctaaatatgtccaaggggatggaagagtgggaggcaaggggtgatcagatcatttataatacactcaacctggtggaatagtattagaagcattagtaattacattttagagacatggagaaaagctcatgattttaaactaactgaaaaaagcatgaaaaattgcatctggatgctgttgaggaattattgctaattttttgagatgagaaattgtattaaagtctttttcaaaaaagagtccttaacttttagagatgcacacaagtgtttatgggtgaaattaaataattcagtagagacattaagtgggaaatagaggaaatattgaccatgggttgctaatagttgaagccaggtgttgggtataaggaggttctcgctgcttttatttgaaaagttgctttttatttgaaaatttaataataaagagtttttaaatttgtatctgtattttaatataaatataaatgcacttaatataaatataaaatatgtataacatttagatagagaaaagctaaaagattactgtggttgattctaggaaactgcattgcagatagtttcatgggtttttttttcctttttcttccactttttgtaccatctatgggtttttgtttgttttttgttttttgggttttctttcttgtttgtgttttgttttttgagacggagttttgctcttgttgcctaagctggagtgcaatggcacagtctcggctcactgcaacctctgcctcctgggttcaagtgattctcctgcctcagcctcccaagtagctgggattataggcatgtaccaccgcccggctaattttgtatttttagtagaggcggggtttctccattaataaattcctggcacaaatttagtgttcaattttgatatatgttgttataaccattgtgaggatactcaggctcaggtttgtgtgggtggaaaacatggtcttcagaaagaaattatgagtgcaagacaggaggaaatccatcagaggccccagctgaggactgaccacggcttgttatttctcttgccttgcctctggcaatcacagcctcacagagcctgcaatccttgctttgtgagtttatagctcagtccagagaatggctaagaaagtttaggattctttcaacacccactccacaaaaaaaaaaaaaaaaaagaaaagaaaaaaaaattaatttttgaaatacttgaggtagaaaacttgaggcagaaaaaaattgagccaaaaaaaaaggaaaattgaaccacgtgaaagcaggcaagaaagcttgcattgctcagggcatcccaggcccagagggcgctttggagggagctgggtttcctgagaggaggcagggtgggtgacggacctgtgctggagagccttgaggaccactgtgggttgggaatgggggcagtggattggggttcaaaacccctgggaatgagaaatgggctcaggaaggctagggtggattctttcatcttcctctttgcttggctttattttcacaaaggaaggcagggcaggaaatagtctcagcccaacttcagtgtggttcttcttagtgctcaggcttacctggcacttgccacacctctgggatgggagcacctactatccatcagccacgtgccagtctccacaaagtctgctcctgaaccctgctcctcagctggccccacttcacagatggggacataggcagcttggctttggaatgaaggaatgaagtcaggaatgaagtcctggctctgcacttggtgactgtgcactgggcttgctaagtctgtttcctgcttttaaaatggagattgtccatcagcctttgaagccatgtaatgggtatgtgtcaactttctgcaaggattaaaggcatggtataggaagtcccaaacacactgcctgacccatctttgatgctcaagaaacgatatatgttgttgtcatgaggaaactgagcctcagaaagtttggatacctgaaaaacactgactactattgaatgaggttgtgaagaatccagagctgtaggggcaggaaagcaaagaacgtattagagctgacccagtcaggacgatcgtctatccccttcctcaccccaccccatcccaggaggaagcctgcccggccctaggcagctatggcacagtggcaatgtcaggtatggttctccctagccagagaccctagcctcaaaaaacctccttcttgggatccaggcatccaactgctcctccccagccccagcctctgacccagtatcctgagtccagagacgtttggaaccagcacctgtaatggaggagctgaacaaggaggggaacttctgctgctccacagcaggtcacggtcataggagggagtggaaccagaatggcagaatccagatcttggctgcctttcccaaggacttgttctgattcctagcagcacagcccaggcattccgagaagttgggctctctggcatcactcactctgcccagaagagccaggggaaagttggggcttctagctgaaccttgatcccacctgccctcttgaggggctcagaatctgctggctgcttcacaggtgggattctcacggcacgctggccacagctgatgcttcgaccccctcatcttgtttggccaaagtgcagctttttagcttgtgagtaaggaagaaaagctgtatcatatgtctttaaacatcttcctagaccacctttgttttccccttaaagtgtgcttaaggagaaaatggaaagtctcttttcagtgtgttgtattttgtttattgcaaaatacaacacactgaaagaacaatgtctgggttagcaaagtattagatttaatttcccacattactaccatccaggctgagaaagagaaccccagaagcctctctcatgtcccttcctgaccacagcctcttcctctcccacaaagaaccacaagtctgacttctatggtgatcacttccttgcttttccctatagtttgactgaatctaaatatcatccctaaacaatatagcttagctttgcctatttttgatcttcatatttggattcatactttttggtttggctcaagactaaacttttaaaatgcatccacgttgtaggagatgtacatgcacaaaaattttcacggcaacattgtccaaactagcaacaaactgaaagccacctatgtgtgcattagcagtagaaatggcaataaatcatggactagtcacacagtggaatcctacgcagcaaggagaattagcagtctacagccaaaccaacagcatgggtgagatgcttccagaaatactgagatgaatttagagataaaggcacaattggtccatatctacagggcacttaatccagtgcatctccactagacaaaatttgtatttccatgagcaagagtcatttgcactgctatctgctataacctacggtgctgtaaaattagctcaggcaataaaagggggaggtagccccaaagagtatgccagaaaggactcccagtaatcttcagtgtgtgtctatttcaaagtctttcatcatttttccttgtagttacttagtgtggaatttcagacccctctctatgtccctcttctcccttttcagccttggctttctctgcatccttccccgaagccctgcactcagcctaaactgactttcctgaacactctgggagcatgtgggctcctcccaactccaccatcttcactgccttctgttcctatttccctctgcctggccttggccccaggaaaccttcctatgctcagaccctctgtgcctttgtcttcaaagcccacccactattcactgccacctccatcttggtcaacctggaagactttccccatccttaaaacatcagctcaaacggcagatttttttttttttttttgtgatatcctcttgatcaccctcccccagtttttaaggagacagatatagaggttcaatccttgggcttcccatgacttttcttatattctgttactgagcaataataaacacttctaggaactgtatcttaaacacttttgcctctacagaatctagcacagtgcctagtattggtgaaatataattaacaaagtcttctttcaacacagagattctctccacaaaaggagtagagaaagaacagttttattatggaataagcagtaaaccaaaatatgcagagcattataggccatctactaagaggttgcaagaacagaaagaaatctcccccttttgtatagccaagtagatacaacctgttacatacatgttatcaaggcaaacaataactgttcctcaagtaagaggtcttgccagcaccgtttgccgtacatggttcactctaaatttacctggtaattagggtaaccacttgtgttagctaactggctctacccagaggaaaatcaaatttatctttaagacaaggggtaattttgcagcactgagcaaggctcttcagttaggctcataccttcccacagaaactaagagatagaagcactatctcccttaggtttgtttacatttcaaagagatgacgcccaggtccttgggaaagactagcttagctcataaagctgacaaaaagcctatctagtttcaaaaggatttacacatgttcaaagagaggagaaagtatgtaaaagttttctaggtgggcacggtggctcacacctgtattcccagcactttgagaggctgaggcgggtggatcacctgaagtcaggaatttgagatcagcctggccaacatggtgaaaccttgtctctactaaaaatacaaaaattagctgggcatggtggtgggcgcctgtaatcccagctacttgggaggctgaggcaggagaatcacttgaacccaggaggtggaggtggcagtgagccaagatggcaccattgcactccagcctgggcgatgagagtgaaactccatgtcaaaaaaaaaaaaagttttctaaagtaaatgctctgagaaaaaaacagaaggtgaggagatctctgtccttatttttaacaagaataattaaatgtttttatttttaatttctacttagactagaatatggtacagatgctcaaaaatattgacttaattaataattaaacaattaacaagtctatctctttcctaattattaaaaaaattatgaagtatttcccttcaaaatttcacagtttactttgacataaataaataaataccataaagtattttgaagaggaaagggaagggtggggcttcaaacacaaaataatggcaggttttgtaaaggggcaattcactaaacccaataggattctcctgttgctattttttttttttttttttttttggtggtggttggagggaagggttgggtgtgcatttggcctgtgtgaaaaactggtgggtagacaatgccatgctgttctagatgggctcattgctataggaatagatagtcactgaatttacttgcataggggatggggtataaaatgtttccttatagctccttaacatggaattcagcacccctctccccatcagccttagctctctctgaatcccaagggatatgtgttataaaaatagctgccatcataaatgagaaaaccatcaggacctggtaaaacttggcttcccaaacagtcaggggtctgggcatggcagccgactgagaacctttctatctagtacaaggtaaacaagatttgccaagacttgatcaactgatccagctccctatctccaaacagaacctcatctgaatgctcacaggctgtgccccagaagtgtagaagtgtcatgttccctgggcaggctgctgggcagcctctctgttgacaatagaccacacttttgctgacctaggactcatgttgctctttaagactgcttccttggccaggcatgatggctcacacctgtaatcctggcactttgagaggcccaggcaggaggatctcttgaggccagatgttcaagaccagcctggtcaacatagtaagaccccatatctaccaaaaatagctgggcatggtggtgcacacctatactcccagctacttaggagactgaggtgggaggattgctacatcccaggagttcaaggctgtagtgagctatgatcatgccactgcactccagcctgggcaacagagcgagatcctgtctcaaacaaacagtttccttctgtttgattcttgctgaaaaattgagcatgccagagctagcaaggctcttagaggtggtctggtccaatgctttaatttcatacatcaagaaactgaagcagagcagagtgacctgcccagggtctctcagccattcatgctcagaaatgtatgggctcctgtgaaacatgtggctcttaaaagcactatcatatatttgaaggcagaaaataggctaaaccttcagccttcagacttttcctctccagagaaaatgaccccagtttcctcactatggttgctgggagctagattcctggggatctggcagtgtggaccacctagtggtggctagaggagcaaataatatcccgcattccattttccactcaccaatccctgaggggcagcctgctgggttatgagcccacagggggagaaccccaacgaattcagagatgcatcatggaccagtttctcttaaggggcctgggtctactattttcagttctacttcgagagaagtggcctgcaatatcctgcagatttcccctccagggagaaaagcattgtgcggtgcaaggagcacaggctttgtggaaagaggcatctggggttgagatcctggctcttttgcttccttgaacaagttaaccaaatctctgggcctctgctggttaatttataccatggggatcatcatttctccagtggggttgtgaagagaatgtggtgggatcttgtgtgtggagcatgacacttagcaggcatccgggaatggcagcctcctccctttctaaactggggctttctgagggtgacttcagattccacaatgtcaacagcacaatggcatcctcataaggaaagtttgggttggggctcctcaagcaattctctactctcatttggtacaaagaaaaaaattaagcctcacaattttcttggcaccagactgaacctcaaacccagtcttcacttttactaaaaagccataaacagagaccaggagggtaaaaactaccagaagatacactggatttagaaaacagtaagctggatgtgaagccaaaagagagggagaaagcaacataaaagaatgtcagattgaagaagaaatggattctggccacctaaagaagaagggaagtatagaaaagggaagctgattggaaacaatgggtaatgatgagtgtccctcccctaaaaagttaaaaaataaatgagcaggcatgagaaaaggaagtaggccagaggaagtagaccaggatcagccacggatgtgagtggggaattttacaaatttctacttgggtgtgaaacatgtgtacactcaaagctctgtctaggttcaccgaaactctcccagctcaaggccatttagtaaaaccttggcttaactgaatagtgggtaaggaggcctgaggatggcccagagaaggtttaaattcttactggctgctgcactaagttgtaaagtgcagccttagttgatgatgcctttattagtgcaacatcccagaatgcatgcgtcttacaacccttgtggatatgaaacccagctgggtagacactgcaaagtcttctcatttgattcttccacttggtttctagtgtccctcagggacaaaggaaagccatggtccagtctaagatgaaaaccaattagacctctggcaggcctttgaccaccctgagggccacccccaaggcacggccacactcgcatctgctgccaggaggccctatcttaggctcagctcccaaaccttgaacatcttggaggatccacaaataggaggtacactcagctcaggctcagtcttccttaaaaagcgccttgctaaagctatcaagttcactggaatacttctgcgaaggacacagcttcagcgatgtcagattttttatgtaaatggtgccttcacatgctctggggcatttggctaccaagggcggtttgaactagctccagcacacagaatacaagtctcttcagaaccaggcaaaaccctatgttgcccaatgactcctgctgtttcttgagatttgcacagaagacagagctgcaattacccacgctgatatctatttcatgaccacatatgttcaaaagccacatgtgagaggtatggttgaaatgagaggttgtgtttgccaagttgtcttctgactgtggagagctggtgagcctcttctcatctcctggggctccatattatagagctgacgaatctcttttcttgctccttgaagtctttcattcacttatttattaattcatttaacacatcaggcacctactaacttctcagagttcaaggcttcctagcctatgatcagaaaggacacctgtgtgctcatgagcacccctgggatcagggtgactgatagggtgctgtcatcactactagatgaaactctttggaacaaaggtctaggatataatatttatccttctgcaaatattcatatggcattcgctgtgtaccaggccttctgttgaacattggcctgcagagctgaagaggacgtgggtcttctcttaggaactcctagtcttgggaaaaaggaatggggaagggctgtaatgtgaagttaaaaaaaagtgctgaggccgaacacatctgggccctcactgcagccctattgcacacacactgcatcacctcgaacaagttattcaccctctctgaattcatctatttgcccataaaaatagagatgatccttttatatgttggctaccacttattatgtgcagcatgcttgttattgtactaagttactcattttttagtttttcatataactcatatttttgttgatttttattttaaaattccaaacgaattaattaaactattttccaaaatacgtagtatgtgtacatggtaaaaaacatttccaaaagtttaccataaaaactgtctcctttcccatatcctcaatccttcagaatccctctccagggataattaccaccaccagtgtattccttaagagatatttaaactttatacaaaatacgcacacatcattcttttccacaaatgacagcatactatgtacatggtacctcacctagcttttttcacttaccagtatatcttagagattgttgcatgacagaatatacagatctgcctgtttttgttgtttttcccaagtttccaagagctagaaactgtttggtttttaagcagctgcttggtattccattaattggacttatcgtgctttgatctgtccctagtgatggacattggggttgtttcccttcatttatatttgaagcttgttgcagtgacagtgtacacacttgcttgagcatgtgtgtgtctacgataaatcccaataagcaatttaaagtaatttaattcttatgctctcaacaaacctaagaggttatttttttagaggaggaagctaaggctgttgttttgatgcctttttaccactggacttgggactcaactgtttgatagtcatcgagtccttgactcttctcccctgccaggatttggcccctaagcccagggcccgagtctcctccatcttcaagggagagtgggaacagcacaggccttggcctcagtcctgctccccctgcttctagctgtgggatgggccaggtgcttcacccagctgtgcctcctgtgaggcacagtgtgtgcaaaatggaaatgtgaacatgaagatcaaaaatgtccctcaatgaccagtgctgttcctcagagtcacgtgggaactcatagaaagcgatattggtactgctctttcctctgtagcatggtccagatggctcatagcagggaccatgatatgctgggtgagcacccactgcatgcacccactgtgccagcactgagagactcctgtgggagccacagcaattctagggtcttcactggggactctgagacagcagggagctaggatgagggctgcagagtgttcgtctgccctcactgagcagaccccctggatggcagggagcagtcccaagccagatggatgcccataaccagccatttggctctcaatacataatatcaccacgtatcaggcaaaaccatcctgcccagagcattatctgaatttgcatcccatctgcagaagatacattcacccacttcttccattctgtcttaatcaaagtctttatgtgaattttccccattgagaagacaagccccttcctggcttagactgtacctgactgatcttttcatgagctccttgccaagccagaccacccccagcttatatggagacttggtgcaaattagagatgcccctgtgcacgtggcagccctgagcccaagcacccagtaaggcaaagggcctgatttgggacccctctgccactccaccaggcaatcagttgcttatttctaactttcccttccttctccacatttgtcccattccttcctctcatcatgaatatccccagaggcattcagcagtgcagtgaattaaatatagaacttttttttttcagaattgcagaacggattagatcaatattaatccaaacagagcaatgagcctgacagtttagtaaaagctcaataaagggtggcttacctcccccaaaataatctgaaaagaaagcatgtcttatttcagggggaaaaaaaaataaagtgacctttaaagaccaaattcccaggatacccagggtggaggtggaacatgggagtccacaggcagcctggatgtttccaaagatccaaagggcttttgcttcctcacataatgcaggaaacaaattgaacatgtattaagtgcttgctgtatgtgacacactgtgccaggtgctccccttaaaacagttctgtgggcaggcatgagaatgaatcccgatcttacagacaaggaatgttaggctcagaggtttcaagctcacccatcactcagccagagaggacagatgcaggattcaatctcgggagtgcccgagtccacagaagttcctgtgctgaaggaccgaccacaggcacataaagagatgcgagacaatttttactggatttggccacctctcgaggtcggctttgccagctcttctcactgggggaaggggagggagaaagtagctagctccagggtccctaacatagaaccaccaaggacttgactatttttactcatacagcagcttgtctgggaagatcatgctctgtgacaagctgcaggcactaagtagcaatttctgtttcccacatattagcttgagtcatataaaactgacatggatgtggctcaaaaatagctgtatgtcagccattttataccatttgacttaaatgttattaattaacgtcacagccagagattattctctgagaaaagggcattgtagcctgaagcagagaaagcatacacgttccctggggttgagaactcatcacagcctgagacagcttaggttgtaaagccccggcccacttatcccaggagagtctgggtgagatgcaggccccaaagcagaggctgggaagcgagaagtgacacaccctggctgggtgggccctcatcttggtgagacaccacctgggtaaaaccatcatggaaagggtgtagtggggcgtggaaactccctcggttaaagcgtgagctttgctgtaagttgtggtaaggagggaggcagtgacaaccaggaggcctgttttgagggtttctgagggacccatctgtggtatcacgaggagacgcccagaggagccgtgtgaaagggctgcctcccagccggctctggagtgaatgagcagcaagtcctggctgcgaaaagaaggggagtgcagcctgcagaagtgtcttcttttttcaattcctgctcagaaggaaacaggagataagaatagtggggaagtccaaaccaaagtgaactatagggctggtaatcgtagggggaattagtcacccggagactagcccagcagactaacggagccccatcctccatcttgaatcagtcagcccctctatgactgcagagtcctgaatgatggcaacaccttctcttcacttagcgttgtaggatgaccaacagtcctgatttgcctgggactgaggggttcccaatagatgggactttcagggctaaaaccaggaaagtcctgggcagcccaaaacaaggtagtcactctagagtgtatgactctgtctgatacctgctaagaaagagaaggacttgttgattataaggagaagaggaggtgaaatggttctcaaaaaacaaagatgagggcttccgggtgctgttctgcccaaggctctgggtctgaggcttctctctccaggcctagcttcatggaaaagtaaggggccagagggtggaaaaggtggaaacaaaggaagaggatggagaattgctttggggaagtttggactggaagtgtgaattacagctgcacccccaattcaccccatctcacccccctccccctcctgctcatggttctccctttctcatccacacattggtcaaactagctagcttttggagagattttgggcagtaaaagtaaaacagatctgtctcaagcttcaaaaagcctagagctggctgggcgctgtggctcacgcctgtaatcctagcattttgggaggctgaggcggaaggataatctgaggtcaggagtttgagaccagcctggctaacatgatgaaaccccatctctactaaaaatacaaaaattagccaggcgtggtagtgcacgcctataatcccagctatttgggaggctgaggcaggagaatcgcttgaaccccaggggacagaggttgcagtgagctgagatcgcaccactgcactccagcctgggtgacacagcgagactccatttaaaaaaaaaaaaatgcctagagccaaatgctcacagagccatttactgcatggctttgggcaagtcaaaggagtccgcctctcctgtcagaagagtctgttgcagtcttcatcacaagactgttgtggggattaaacaagatggcaagtgggaagttgggaaatgtagtgtgcacccaaccaatatttgtttcttcctgcctgcctacatatgaggccacacagaattccaactttgtttctctgataactaacacagttacttgtttttctttctgatccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttccatctgtgtaatcttctttctcctattcaattatttcctgtggttaaatt

SEQ ID NO: 12 - RNA, 20 nt, synthetic construct
  source 1..20: mol_type = other RNA
ctggcccgtgctccgcaatg

SEQ ID NO: 13 - RNA, 20 nt, synthetic construct
  source 1..20: mol_type = other RNA
tgacgccatgctgcacggtc

SEQ ID NO: 14 - RNA, 20 nt, synthetic construct
  source 1..20: mol_type = other RNA
aaagctcacgctttaaccga

SEQ ID NO: 15 - RNA, 20 nt, synthetic construct
  source 1..20: mol_type = other RNA
cacgatgtccccaatataac

SEQ ID NO: 16 - RNA, 20 nt, synthetic construct
  source 1..20: mol_type = other RNA
gacccatctgtggtatcacg

SEQ ID NO: 17 - RNA, 20 nt, synthetic construct
  source 1..20: mol_type = other RNA
gtaaatgtcggtgacactct

SEQ ID NO: 18 - RNA, 20 nt, synthetic construct
  source 1..20: mol_type = other RNA
atggggctccgttagtctgc

SEQ ID NO: 19 - DNA, 2037 bp, synthetic construct
  source 1..2037: mol_type = other DNA
gctgaggcggaaggataatctgaggtcaggagtttgagaccagcctggctaacatgatgaaaccccatctctactaaaaatacaaaaattagccaggcgtggtagtgcacgcctataatcccagctatttgggaggctgaggcaggagaatcgcttgaaccccaggggacagaggttgcagtgagctgagatcgcaccactgcactccagcctgggtgacacagcgagactccatttaaaaaaaaaaaaatgcctagagccaaatgctcacagagccatttactgcatggctttgggcaagtcaaaggagtccgcctctcctgtcagaagagtctgttgcagtcttcatcacaagactgttgtggggattaaacaagatggcaagtgggaagttgggaaatgtagtgtgcacccaaccaatatttgtttcttcctgcctgcctacatatgaggccacacagaattccaactttgtttctctgataactaacacagttacttgtttttctttctgatccaggccttcaccatggaactgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcgagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctactagtatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcaca

SEQ ID NO: 20 - DNA, 2037 bp, synthetic construct
  source 1..2037: mol_type = other DNA
ggccgctgagaactcatcacagcctgagacagcttaggttgtaaagccccggcccacttatcccaggagagtctgggtgagatgcaggccccaaagcagaggctgggaagcgagaagtgacacaccctggctgggtgggccctcatcttggtgagacaccacctgggtaaaaccatcatggaaagggtgtagtggggcgtggaaactccctcggttaaagcgtgagctttgctgtaagttgtggtaaggagggaggcagtgacaaccaggaggcctgttttgagggtttctgagggacccatctgtggtatcacgaggagacgcccagaggagccgtgtgaaagggctgcctcccagccggctctggagtgaatgagcagcaagtcctggctgcgaaaagaaggggagtgcagcctgcagaagtgtcttcttttttcaattcctgctcagaaggaaacaggagataagaatagtggggaagtccaaaccaaagtgaactatagggctggtaattctagattcgaactgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcgagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctactagtcgtagggggaattagtcacccggagactagcccagcagactaacggagccccatcctccatcttgaatcagtcagcccctctatgactgcagagtcctgaatgatggcaacaccttctcttcacttagggttgtaggatgaccaacagtcctgatttgcctgggactgaggggttcccaatagatgggactttcagggctaaaaccaggaaagtcctgggcagcccaaaacaaggtagtcactctagagtgtatgactctgtctgatacctgctaagaaagagaaggacttgttgattataaggagaagaggaggtgaaatggttctcaaaaaacaaagatgagggcttccgggtgctgttctgcccaaggctctgggtctgaggcttctctctccaggcctagcttcatggaaaagtaaggggccagagggtggaaaaggtggaaacaaaggaagaggatggagaattgctttggggaagtttggactggaagtgtgaattacagctgc

SEQ ID NO: 21 - DNA, 751 bp, synthetic construct
  source 1..751: mol_type = other DNA
ctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 22 - DNA, 996 bp, synthetic construct
  source 1..996: mol_type = other DNA
ctgacctcttctcttcctcccacaggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcgagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggct

SEQ ID NO: 23 - DNA, 20 bp, synthetic construct
  source 1..20: mol_type = other DNA
tgattcagggaactgatcca

SEQ ID NO: 24 - DNA, 20 bp, synthetic construct
  source 1..20: mol_type = other DNA
actatagggctggtaatcgt

SEQ ID NO: 25 - DNA, 20 bp, synthetic construct
  source 1..20: mol_type = other DNA
gtcaccaatcctgtccctag

SEQ ID NO: 26 - DNA, 20 bp, synthetic construct
  source 1..20: mol_type = other DNA
gctgaggcggaaggataatc

SEQ ID NO: 27 - DNA, 20 bp, synthetic construct
  source 1..20: mol_type = other DNA
gccaggttcaggaggtaaat

SEQ ID NO: 28 - DNA, 20 bp, synthetic construct
  source 1..20: mol_type = other DNA
gctgggaagcgagaagtgac

SEQ ID NO: 29 - DNA, 20 bp, synthetic construct
  source 1..20: mol_type = other DNA
gcctggagagagaagcctca

SEQ ID NO: 30 - DNA, 22 bp, synthetic construct
  source 1..22: mol_type = other DNA
gatcctctctggctccatcgta

SEQ ID NO: 31 - DNA, 22 bp, synthetic construct
  source 1..22: mol_type = other DNA
tcttaggatggccttctccgac

SEQ ID NO: 32 - DNA, 22 bp, synthetic construct
  source 1..22: mol_type = other DNA
ctagttgccagccatctgttgt

SEQ ID NO: 33 - DNA, 23 bp, synthetic construct
  source 1..23: mol_type = other DNA
gagaaagggagaaccatgagcag

SEQ ID NO: 34 - DNA, 22 bp, synthetic construct
  source 1..22: mol_type = other DNA
ccctggaaggtgccactcccac

SEQ ID NO: 35 - DNA, 22 bp, synthetic construct
  source 1..22: mol_type = other DNA
ctagttgccagccatctgttgt

SEQ ID NO: 36 - DNA, 22 bp, synthetic construct
  source 1..22: mol_type = other DNA
atttgtttccacattgcggagc

SEQ ID NO: 37 - DNA, 22 bp, synthetic construct
  source 1..22: mol_type = other DNA
ccctggaaggtgccactcccac

SEQ ID NO: 38 - DNA, 20 bp, synthetic construct
  source 1..20: mol_type = other DNA
gattgggaagacaatagcag

SEQ ID NO: 39 - DNA, 20 bp, synthetic construct
  source 1..20: mol_type = other DNA
tcttgggaagtgtaaggaag

SEQ ID NO: 40 - DNA, 20 bp, synthetic construct
  source 1..20: mol_type = other DNA
ccagataaggaatctgccta

SEQ ID NO: 41 - DNA, 22 bp, synthetic construct
  source 1..22: mol_type = other DNA
gctgtcatctcttgtgggctgt

SEQ ID NO: 42 - DNA, 21 bp, synthetic construct
  source 1..21: mol_type = other DNA
actcatgggagctgctggttc

SEQ ID NO: 43 - DNA, 26 bp, synthetic construct
  source 1..26: mol_type = other DNA
cctgtcatgcccacacaaatctctcc